(19) United States
(12) Patent Application Publication
(10) Pub. No.: US 2023/0140435 A1
(43) Pub. Date: May 4, 2023

(54) COMPOSITION AND METHODS FOR IMPROVING HEART FUNCTION AND TREATING HEART FAILURE

(71) Applicant: The Trustees of the University of Pennsylvania, Philadelphia, PA (US)

(72) Inventors: Benjamin L. Prosser, Philadelphia, PA (US); Kenneth B. Margulies, Villanova, PA (US); Alexey Bogush, Elkins Park, PA (US)

(21) Appl. No.: 17/906,522

(22) PCT Filed: Mar. 16, 2021
(86) PCT No.: PCT/US2021/022561

§ 371 Date: Sep. 16, 2022

Related U.S. Application Data
(60) Provisional application No. 62/990,147, filed on Mar. 16, 2020.

(51) Int. Cl. C12N 15/113; A61K 38/53; A61P 9/04; A61K 45/06; C12N 15/86; C12N 15/85

(52) U.S. Cl. CPC C12N 15/1137; A61K 38/53; C12Y 603/02025; A61P 9/04; A61K 45/06; C12N 15/86; C12N 15/85; C12N 2310/531; C12N 2310/533; C12N 2310/122; C12N 2840/20; C12N 2750/14143; C12N 2320/31

(57) ABSTRACT

A recombinant viral vector comprising an expression cassette which comprises a coding sequence for an shRNA inhibitor of vasohibin (VASH)-small vasohibin binding protein (SVBP) complex operably linked to regulatory sequences which direct expression thereof is provided. Further provided are compositions containing such viral vectors formulated for delivery to a human patient. Also provided are methods using these vectors and compositions for improving or stabilizing cardiac function.

### STATEMENT REGARDING FEDERALLY SPONSORED RESEARCH OR DEVELOPMENT

This invention was made with government support under HL133080 awarded by the National Institutes of Health. The government has certain rights in the invention.

### BACKGROUND OF THE INVENTION

Heart failure is defined as a failure of the heart to provide adequate blood flow to the organs and tissues of the body. Currently, there is no cure for heart failure, and existing therapies have shortcomings. Other approaches used in clinical settings to boost cardiac contractility (cardiac “inotropes”) have a marked limitation in that they are energetically unfavorable. For example, inotropes often target improved calcium cycling or increased force generation, which both require increased energy usage to fuel these ATP-dependent processes. In heart failure with reduced ejection fraction, the existing therapies for augmenting the heart's contractility have been associated with no survival benefit, with an increased risk of ischemia or arrhythmias, and are thus viewed as palliative. In contrast, targeting microtubules to lower internal resistance should be energetically favorable. This does not cause the muscle cell to intrinsically produce more force or cycle calcium faster, but simply lowers the internal resistance that normally opposes that force. This should allow the heart to do more work for the same amount of energy usage, a distinct advantage over other inotropic approaches.

Heart failure with preserved ejection fraction (or HFpEF), currently has no approved therapies, even though it is estimated to represent almost half of all heart failure cases. Many cases of HFpEF exhibit slowed relaxation of the heart muscle that contributes to abnormalities of pump function. Slowed myocardial relaxation in HFpEF may also be due to increased internal resistance attributable to microtubules (MTs), and targeting MTs might enhance relaxation rates without increasing energy usage.

Along with its well-defined transport functions, the MT network serves multiple mechanical roles in the beating cardiomyocyte. MTs function as mechanotransducers, converting changing contractile forces into intracellular signals (1, 2). MTs also act as compression resistant elements, which provide a mechanical impediment to cardiomyocyte contraction (3, 4, 5). As such, they bear some of the compressive and tensile load of a working myocyte. During the contractile cycle, Ca²⁺ mediated actin-myosin interaction first shortens repeating contractile units called sarcomeres, which are then stretched as the heart fills with blood during diastole.

Although an isolated MT would present minimal resistance to myocyte motion, the stiffness of the network within a living cell, with microtubule associated proteins and other cytoskeletal elements, can change by orders of magnitude (6, 7). It is in this context that MTs act as viscoelastic resistance elements that can impair sarcomere shortening and thus cardiac function, particularly in disease states associated with MT proliferation (6, 8, 9, 10). Post-translational modification (PTM) of MTs (11, 12) modifies their mechanical properties and binding interactions. Detyrosination, a PTM of α-tubulin, has recently been shown to augment MT-dependent mechanotransduction in dystrophic cardiac and skeletal muscle (12). This specific PTM is also increased in animal models of heart disease (1, 13, 14).

In failing myocardium, the maladaptation of a cardiomyocyte to pressure of an inducing overload was correlated with an increased density and detyrosination of the microtubule network (for reviews, see 15, 16). A proliferated and modified MT network may thus mechanically interfere with contraction in failing myocardium.

There remains a need for treatment of heart failure, and particularly treatments which minimize the increased risk of ischemia or arrhythmias associated with current palliative efforts which require significant energy.

### SUMMARY OF THE INVENTION

Provided herein are compositions and methods useful for treating patients with heart failure and/or useful for lowering cardiac stiffness and for improving contractility. Our studies suggest that genetic inhibition of VASH-SVBP complex, a predominant tubulin detyrosinase in cardiomyocytes, or/and in addition to activation of TTL (tubulin tyrosinase), is sufficient to improve relaxation and contractility of cardiomyocytes from patients with HF. The composition includes a replication defective vector comprising an expression cassette with a coding sequence for an inhibitor of vasohibin (VASH)-small vasohibin binding protein (SVBP) complex operably linked to expression control sequence and is useful for treating cardiomyocytes in patients with heart failure. The method involves delivering the composition comprising of replication-defective vector, diluent, suspending agent, and/or optional preservative, for transducing human cardiomyocytes.

In certain embodiments, the replication-defective vector encoding inhibitor is a short hairpin RNA (shRNA) and is selected from targeting VASH1, VASH2, or SVBP. Furthermore, in certain embodiments, the shRNA sequence is selected from VASH1 sh1, VASH1 sh2, VASH1 sh3, SVBP sh1, SVBP sh2, or SVBP sh3.

In one aspect, the replication-defective vector encoding inhibitor further comprises of at least a second VASH-SVBP inhibitor operably linked to regulatory sequence which control the expression thereof. In another aspect, the replication-defective vector further comprises of a coding sequence for tubulin tyrosine ligase (TTL) operably linked to regulatory sequences which control expression thereof.

In one embodiment the replication defective vector is a viral vector, and is selected from lentivirus vector or adeno-associated viral vector. In certain embodiments, the adeno-associated virus capsid is selected from clade F, or is further selected from AAV9, AAVhu31, AAVhu32, AAVhu68, or a variant which targets cardiac cells.

In one embodiment, the replication-defective vector composition is used in treating failing cardiomyocytes. In another embodiment, the method is used in patients with heart failure with reduced ejection fraction (HFrEF) or preserved ejection fraction (HFpEF).

In one aspect, the method comprises co-administering replication-deficient vector in a combination therapy. The combination therapy may be selected from: calcium desensitizer, myosin inhibitors, a small molecule VASH inhibitor, a small molecule small vasohibin binding protein inhibitor, tubulin tyrosine ligase, and/or parthenolide therapy.

Other aspects and advantages of the present invention will be apparent from the following Detailed Description of the Invention.

### BRIEF DESCRIPTION OF THE FIGURES

FIGS. 1A to 1M show identification of the primary cardiac detyrosinase and development of tools to probe the tyrosination cycle. FIG. 1A shows a schematic of proposed microtubule tyrosination cycle in cardiomyocytes. FIG. 1B shows average traces of electrically stimulated [Ca²⁺ ] transients from failing human cardiomyocytes infected with a null (grey) or TTL-encoding (blue) adenovirus. Traces depict change in signal normalized to resting fluorescence (F₀) or resting length. FIG. 1C shows average traces of sarcomere shortening (bottom) from failing human cardiomyocytes infected with a null (grey) or TTL-encoding (blue) adenovirus. Traces depict change in signal normalized to resting fluorescence (F₀) or resting length. FIG. 1D shows quantified kinetic parameters of calcium transients, measured as rise time. FIG. 1E shows quantified kinetic parameters of calcium transients, measured as decay time. FIG. 1F shows quantified sarcomere shortening, measured as contraction time. FIG. 1G shows quantified sarcomere shortening, measured as relaxation time. Statistical significance determined via two-sided t-tests with Bonferroni correction for multiple comparisons, *** p<0.001 vs. null. FIGS. 1H and 1I show validation of TTL-E331Q construct. Representative western blots were used in quantification of TTL, detyrosinated tubulin (dTyr-tub), α-tubulin (α-tub), and GAPDH levels in whole cell extracts from isolated rat cardiomyocytes expressing null, TTL, or TTL-E331Q constructs. FIG. 1H shows fold change in TTL expression. FIG. 1I shows fold change in expression levels of dTyr-tub over α-tub. Statistical significance determined via analysis of variance (ANOVA) with post-hoc Bonferroni test from duplicates of N=3 rat hearts, ** p<0.01, *** p<0.001 vs. null. FIG. 1J shows expression profiling of VASH1, VASH2 and SVBP genes via RT-qPCR of patient myocardial samples. Statistical significance determined via ANOVA with post-hoc Bonferroni test, ** p<0.01, *** p<0.001 vs. NF. FIG. 1K shows expression levels of Vash1 and Vash2 in isolated rat cardiomyocytes. FIG. 1L shows validation of knockdown (KD) via RT-qPCR quantification of Vash1, Vash2, and Svbp in rat cardiomyocytes. FIG. 1M shows quantification of detyrosinated tubulin, α-tubulin, and GAPDH levels from isolated rat cardiomyocytes treated with different concentrations of adenovirus encoding SVBP or VASH1 KD constructs, and presented as fold change from scramble (scram) control. Statistical significance determined via two-sided Student's t-tests for RT-qPCR and ANOVA with post-hoc Bonferroni test from N=4 for western blot, *** p<0.001 vs. scram. Data are presented as mean±s.d. if not otherwise specified.

FIG. 2A to 2C show that TTL tubulin sequestration, but not detyrosination, depolymerizes microtubules in healthy cardiomyocytes. FIG. 2A shows quantification of detyrosinated tubulin level found in the polymerized fraction as measure from representative western blot of free and polymerized tubulin from isolated rat cardiomyocytes transduced with adenoviruses encoding null, TTL, TTL-E331Q, scram, VASH1 KD and SVBP KD. FIG. 2B shows quantification of free to polymerized tubulin ratio as measure from representative western blot of free and polymerized tubulin from isolated rat cardiomyocytes transduced with adenoviruses encoding null, TTL, TTL-E331Q, scram, VASH1 KD and SVBP KD. Statistical significance determined via ANOVA with post-hoc Bonferroni test from duplicates of N=7 rat hearts for null and TTL and duplicates of N=4 for TTL-E331Q, scram, VASH1 KD, and SVBP KD, *** p<0.001, ** p<0.01 vs. null or scram. FIG. 2C shows correlation between detyrosinated tubulin levels found in the polymerized fraction and the ratio of free:polymerized tubulin.

FIG. 3A to 3K shows reducing detyrosination speeds contraction and relaxation of failing human cardiomyocytes. FIG. 1A shows an example of sarcomere shortening trace from myocyte responding to electrical stimulation and corresponding first derivative identifying key contractile parameters collected. FIG. 3B shows average traces of sarcomere shortening from cardiomyocytes isolated from non-failing (NF) donor hearts, with or without VASH1 KD. FIG. 3C shows average traces of sarcomere shortening from cardiomyocytes isolated from hearts from patients with HFrEF, with or without VASH1 KD. FIG. 3D shows average traces of sarcomere shortening from cardiomyocytes isolated from hearts from patients with HFpEF, with or without VASH1 KD. FIG. 3E shows contraction times quantified from cardiomyocytes isolated from hearts from patients within NF, HFrEF, and HEpEF, with or without VASH1 KD. FIG. 3F shows relaxation times quantified from cardiomyocytes isolated from hearts from patients with NF, HFrEF, and HEpEF, with or without VASH1 KD. FIG. 3G shows contraction times quantified from cardiomyocytes isolated from hearts from patients within NF, and HFrEF, with and without TTL or E331Q overexpression. FIG. 3H shows relaxation times quantified from cardiomyocytes isolated from hearts from patients with NF, and HFrEF with and without TTL or E331Q overexpression. Statistical significance for VASH1 KD determined via two-sided t-tests with Bonferroni correction for multiple comparisons, *p<0.05, ** p<0.01, *** p<0.001 vs. scram of that etiology, and comparison among etiology via ANOVA with post-hoc Bonferroni test, ††† p<0.001 vs. NF, ‡‡‡ p<0.001 vs. HFrEF. Statistical significance for TTL/TTL-E331Q experiments determined via ANOVA with post-hoc Bonferroni test, *p<0.05, ** p<0.01, *** p<0.001. FIG. 3I to 3K shows percent improvement in contractile parameters by each genetic manipulation over null or scram is quantified and grouped by etiology and experimental conditions. FIG. 3I shows percent improvement in contractile parameters by VASH1 KD genetic manipulation; shown as quantified over scram. FIG. 3J - - - . FIG. 3K - - - . In FIG. 3I, statistical significance determined via two-sided Student's t-tests with Bonferroni correction, * p<0.05, ** p<0.01, *** p<0.001 vs. scram and † p<0.05, †† p<0.01 and ††† p<0.001 vs. NF. In FIGS. 3J and K, statistical significance determined via two-sided Student's t-tests with Bonferroni correction, * p<0.05, ** p<0.01, *** p<0.001 vs. null and †† p<0.01 vs. TTL of that etiology. Data are presented as mean s.e.m. in bar graphs (FIGS. 3I and 3K).

FIGS. 4A to 4K show VASH1 knockdown reduces myocyte viscoelasticity and improves contractility independent of changes to the calcium transient. FIG. 4A shows average traces of simultaneously acquired [Ca²⁺]ᵢ transients and sarcomere shortening from fluo-3 loaded cardiomyocytes isolated from the heart of patient with heart failure with preserved ejection fraction (HFpEF). Traces are normalized to baseline fluorescence (F₀) and sarcomere length, respectively. In FIGS. 4B to 4G, amplitude and kinetic parameters from [Ca²⁺]i transients and sarcomere shortening were quantified simultaneously in cardiomyocytes isolated from NF or failing hearts with or without VASH1 KD. Statistical significance determined via two-sided t-tests with Bonferroni correction, * p<0.05, ** p<0.01, *** p<0.001 vs. scram of that etiology, ††† p<0.001 HF vs. NF scram. FIG. 4B shows quantified peak amplitude from [Ca²⁺]ᵢ transients in cardiomyocytes isolated from NF or failing hearts with or without VASH1 KD. FIG. 4C shows quantified rise time from [Ca²⁺]ᵢ transients in cardiomyocytes isolated from NF or failing hearts with or without VASH1 KD. FIG. 4D shows quantified decay time from [Ca²⁺]ᵢ transients in cardiomyocytes isolated from NF or failing hearts with or without VASH1 KD. FIG. 4E shows quantified peak amplitude from [Ca²⁺]ᵢ transients in cardiomyocytes isolated from NF or failing hearts with or without VASH1 KD. FIG. 4F shows quantified contraction time from [Ca²⁺]ᵢ transients in cardiomyocytes isolated from NF or failing hearts with or without VASH1 KD. FIG. 4G shows quantified relaxation time from [Ca²⁺]ᵢ transients in cardiomyocytes isolated from NF or failing hearts with or without VASH1 KD. FIG. 4H shows nanoindentation measurements of viscoelasticity. FIGS. 4I to 4K show stiffness (elastic modulus) of failing cardiomyocytes as a function of probe indentation velocity with or without VASH1 KD. FIG. 4I shows quantification of Eₘᵢₙ. FIG. 4J shows quantification of Eₘₐₓ. FIG. 4K shows quantification of viscoelasticity (EA). Statistical significance determined via two-sided t-tests, ** p<0.01 vs. scram. Data are presented as mean±s.e.m. in FIG. 4H.

### DETAILED DESCRIPTION OF THE INVENTION

Provided herein are compositions which are useful for transducing failing cardiomyocytes and/or treating patients having heart failure, and/or patients with heart failure with reduced or preserved ejection fraction (e.g., HFrEF or HFpEF, respectively) in a Ca²⁺-independent manner. The compositions and methods provided herein may be combined with other therapies, including those which involve Ca²⁺-dependent pathways. The compositions provided herein permit delivery of at least one an inhibitor of the vasohibin (VASH)-small vasohibin binding protein (SVBP) complex. In certain embodiments, the inhibitor is an RNA (e.g., shRNA or siRNA) specifically targeted to VASH1, VASH2 or SVBP.

In certain embodiments, the inhibitor is an RNA specifically targeted to VASH1. In certain embodiments, the VASH1 sequence has the sequence of the transcript provided in GenBank NM_014909.5, which is reproduced in SEQ ID NO: 56. However, other VASH1 transcripts may be selected. The RNA may be targeted to gctgcagtacaatcacacagg (Nucleotide 1-21 of SEQ ID NO: 16; Nucleotide 1747-1767 of SEQ ID NO: 56), gggacacagttctttgaaatt (Nucleotide 1-21 of SEQ ID NO: 17; Nucleotide 1766-1786 of SEQ ID NO: 56), or gggaatttacctcaccaacag (Nucleotide 1-21 of SEQ ID NO: 18; Nucleotide 1879-1899 of SEQ ID NO: 56) in VASH1. In certain embodiments, the inhibitor is a short hairpin RNA (shRNA). However, in other embodiments, the inhibitor may be an miRNA or an siRNA which is directed to a VASH1 target identified herein. Each of these may have an overhang (e.g., 2 to 5 base pairs) at the 5′ ends.

Where the inhibitor is an shRNA, the examples below illustrate a 21-nucleotide stem sequence, the sequence of the loop region (CGAA double underlined in the sequences below), and a second 21-nucleotide stem sequence which is the complement of the first 21-nt sequence. When engineered into the expression cassette, these sequences may also be provided with an overhang at each end. In certain embodiments, the stem sequences may each independently be 19 to 21 nucleotides in length, or longer. In certain embodiments, the stem sequences are about 19 to less than 30 nucleotides in length. Although the loop region illustrated below is CGAA, an alternative loop sequence may be selected. In certain embodiments, the loop may be from 3 nucleotides to 11 nucleotides in length, 4 nucleotides to 11 nucleotides, 7 nucleotides to 11 nucleotides, or 9 to 11 nucleotides in length. In certain embodiments, the loop may be longer.

In one embodiment, the VASH1 shRNA is: sh1: gctgcagtacaatcacacaggcgaacctgtgtgattgtactgcagc (VASH1 sh1) (SEQ ID NO: 16). In certain embodiments, the VASH1 shRNA is: gggacacagttctttgaaattcgaaaatttcaaagaactgtgtccc (VASH1 sh2) (SEQ ID NO: 17). In certain embodiments, the VASH1 shRNA is: gggaatttacctcaccaacagcgaactgttggtgaggtaaattccc (VASH1 sh3) (SEQ ID NO: 18). In certain embodiments, the stem(s) of the shRNA may have less than 100% identity to the sequences provided above, e.g., there may be 1, 2, 3 or 4 mismatches in the above sequences. In certain embodiments, the loop region may be substituted with a different loop region.

In certain embodiments, an siRNA (e.g., a dicer siRNA) is selected which targets VASH1. See, e.g., the transcript identified above. In certain embodiments, these siRNA are 20 base pairs to 30 base pairs in length with an overhang on the 5′ ends (e.g., two bases at each end). In certain embodiments, the siRNA are 21, 21, 25, 24, 25, 26, 27, 28, 29 or 30 bases in length, or longer. In certain embodiments, the siRNA are about 27 bases in length. These siRNA may target the identical regions of the VASH1 identified above for the shRNA, or additionally or alternatively, may target non-overlapping regions.

In certain embodiments, the inhibitor is an RNA specifically targeted to VASH2. In certain embodiments, the targeted VASH2 sequence has the sequence of the transcript provided in GenBank NM_024749, which is reproduced in SEQ ID NO: 58. However, other VASH2 transcripts may be selected. In certain embodiments, the inhibitor is specifically targeted to sh1: gtcaagaaggtcaagattggg (Nucleotide 1-21 SEQ ID NO: 24; Nucleotide 999-1019 of SEQ ID NO: 58), sh2: ggtcaagattgggctgtacgt (Nucleotide 1-21 SEQ ID NO: 25; Nucleotide 1007-1027 of SEQ ID NO: 58), or sh3: gtcaagattgggctgtacgt (Nucleotide 1-21 SEQ ID NO: 26; Nucleotide 1008-1028 of SEQ ID NO: 58), in VASH2. However, in other embodiments, the inhibitor may be an miRNA or an siRNA which is directed to a VASH2 target identified herein.

In one embodiment, the VASH2 shRNA has the sequence of: sh1: GTCAAGAAGGTCAAGATTGGGCGAACCCAATCTTGACCTTCTTGAC (SEQ ID NO: 24). In another embodiment, the VASH2 shRNA has the sequence of: sh2: GGTCAAGATTGGGCTGTACGTCGAAACGTACAGCCCAATCTTGACC (SEQ ID NO: 25). In still another embodiment, the VASH2 shRNA has the sequence of: sh3: GTCAAGATTGGGCTGTACGTCCGAAGACGTACAGCCCAATCTTGAC (SEQ ID NO: 26). In certain embodiments, the stem(s) of the shRNA may have less than 100% identity to the sequences provided above, e.g., there may be 1, 2, 3 or 4 mismatches in the above sequences. In certain embodiments, the loop region may be substituted with a different loop region.

In certain embodiments, an siRNA (e.g., a dicer siRNA) is selected which targets VASH2. See, e.g., the transcript identified above. In certain embodiments, these siRNA are 20 base pairs to 30 base pairs in length with an overhang on the 5′ ends (e.g., two bases at each end). In certain embodiments, the siRNA are 21, 21, 25, 24, 25, 26, 27, 28, 29 or 30 bases in length, or longer. In certain embodiments, the siRNA are about 27 bases in length. These siRNA may target the identical regions of the VASH2 identified above for the shRNA, or additionally or alternatively, may target non-overlapping regions.

In certain embodiments, the inhibitor is an RNA specifically targeted to SVBP. In certain embodiments, the SVBP sequence has the sequence of the transcript provided in GenBank NM_199342, which is reproduced in SEQ ID NO: 60. However, other SVBP transcripts may be selected. In certain embodiments, the inhibitor is specifically targeted to sh1: gacaaagagcagagatctatg (Nucleotide 1-21 of SEQ ID NO: 19; Nucleotide 345-365 of SEQ ID NO: 60), sh2: gcagcagcagtttgatgagtt (Nucleotide 1-21 of SEQ ID NO: 20; Nucleotide 394-414 of SEQ ID NO: 60), or sh3: gcagcagtttgatgagttctg (Nucleotide 1-21 of SEQ ID NO: 21; Nucleotide 397-417 of SEQ ID NO: 60) for SVBP. However, in other embodiments, the inhibitor may be an miRNA or an siRNA which is directed to a SVBP target identified herein.

In one embodiment, the SVBP shRNA has the sequence of: sh1: Gacaaagagcagagatctatgcgaacatagatctctgctctttgtc (SEQ ID NO: 19). In another embodiment, the SVBP shRNA has the sequence of: sh2: gcagcagcagtttgatgagttcgaaaactcatcaaactgctgctgc (SEQ ID NO: 20). In still another embodiment, the SVBP shRNA has the sequence of: sh3: gcagcagtttgatgagttctgcgaacagaactcatcaaactgctgc (SEQ ID NO: 21). In certain embodiments, the stem(s) of the shRNA may have less than 100% identity to the sequences provided above, e.g., there may be 1, 2, 3 or 4 mismatches in the above sequences. In certain embodiments, the loop region may be substituted with a different loop region.

In certain embodiments, an siRNA (e.g., a dicer siRNA) is selected which targets SVBP. See, e.g., the transcript identified above. In certain embodiments, these siRNA are 20 base pairs to 30 base pairs in length with an overhang on the 5′ ends (e.g., two bases at each end). In certain embodiments, the siRNA are 21, 21, 25, 24, 25, 26, 27, 28, 29 or 30 bases in length, or longer. In certain embodiments, the siRNA are about 27 bases in length. These siRNA may target the identical regions of the SVBP identified above for the shRNA, or additionally or alternatively, may target non-overlapping regions.

One or more of the VASH-SVBP inhibitors can be engineered into a suitable expression cassette in which the coding sequence for the inhibitor is operably linked to regulatory sequences which direct expression thereof. The expression cassette may optionally contain more than one VASH-SVBP inhibitor (e.g., more than one RNA targeting VASH1) or a second therapeutic protein (e.g., the coding sequence for a tubulin tyrosine ligase (TTL) enzyme operably linked to regulatory sequences which control expression thereof. Thus, in certain embodiments, the expression cassette may be a bicistronic expression cassette.

Such a bicistronic expression cassette contain regulatory sequences which direct expression of two different ORFs thereon from shared regulatory sequences. In this instance, the two ORFs are typically separated by a linker. Suitable linkers, such as an internal ribozyme binding site (IRES) and/or a furin-2a self-cleaving peptide linker (F2a), [see, e.g., Radcliffe and Mitrophanous, Gene Therapy (2004), 11, 1673-1674] are known in the art. Suitably, the ORF are operably linked to regulatory control sequences which direct expression in a target cell. Such regulatory control sequences may include a polyA, a promoter, and an enhancer. In order to facilitate co-expression from an AAV vector, at least one of the enhancer and/or polyA sequence may be shared by the first and second expression cassettes. In certain embodiments, an expression cassette or a vector genome may contain a bidirectional enhancer. For example, in such an embodiment, a first promoter for a first expression cassette is located to the left of the bidirectional enhancer, followed by at least a first open reading frame, and a polyA sequence, and a second promoter. Further, a second promoter for the second expression cassette is located to the right of the bidirectional enhancer, followed by at least a second open reading frame and a polyA. The first and second promoters and the first and second polyA sequences may be the same or different. A minimal promoter and/or a minimal polyA may be selected in order to conserve space. Typically, in this embodiment, each promoter is located either adjacent (either to the left or the right (or 5′ or 3′)) to the enhancer sequence and the polyA sequences are located adjacent to the ITRs, with the ORFs there between. Alternatively, the opposite configuration is possible, and the expression cassette to the left of the enhancer may be bicistronic. Alternatively, depending upon what gene products are encoded, both expression cassettes may be monocistronic, or both can be bicistronic. In certain embodiments, the expression vector comprises an AAV 5′ITR-cTNT promoter-chimeric intron-TTL-IRES-VASH-SVBP inhibitor-WRPE-Rabbit globin polyA-AAV 3′ITR, wherein the VASH-SVBP inhibitor is a dicer siRNA. In some embodiments, dicer siRNA are 27-mer. In certain embodiments, nucleic acid sequence of nt 1 to nt 4476 of SEQ ID NO: 1 (or SEQ ID NO: 62) or a sequence at least about 80%, at least about 85%, at least about 90%, at least about 95%, at least about 99% identical thereto is selected for the vector genome.

The expression cassette is designed to be carried by a replication-incompetent viral vector or a suitable non-viral vector. In certain embodiments, in addition to or as an alternative to a bicistronic expression cassette, a vector may contain a first expression cassette comprising a VASH-SVBP inhibitor and a second expression cassette comprising a therapeutic gene exogenous to the vector, e.g., a second VASH-SVBP expression cassette and/or an expression cassette comprising a TTL enzyme. In certain embodiments, the expression cassette comprises: cTNT promoter-chimeric intron-TTL-IRES-VASH-SVBP inhibitor-WRPE-Rabbit globin polyA, wherein the VASH-SVBP inhibitor is a dicer siRNA. In some embodiments, dicer siRNA are 27-mer. In certain embodiments, nucleic acid sequence of nt 214 to 4258 of SEQ ID NO: 1 (or SEQ ID NO: 63) a sequence at least about 80%, at least about 85%, at least about 90%, at least about 95%, at least about 99% identical thereto is selected for the expression cassette.

In certain embodiments, the expression cassette is specifically targeted to the heart. In still other embodiments, the expression cassette is specifically targeted to the cardiac microtubules.

In one embodiment, the expression cassette is designed for expression in the heart, including the cardiomyocytes. The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], inducible promoters, tissue specific promoters, or a promoter responsive to physiologic cues may be utilized in the vectors described herein. In certain embodiments, a cardiac-specific promoter is used. In certain embodiments, a cardiac troponin T (cTNT) promoter sequence is used.

Examples of constitutive promoters suitable for controlling expression of the therapeutic products include, but are not limited to chickenβ-actin (CB) promoter, human cytomegalovirus (CMV) promoter, ubiquitin C promoter (UbC), the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86: 10006-10 (1989), the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in certain embodiments include, but are not limited to, endothelin-I (ET-I) and Flt-I, which are specific for endothelial cells, FoxJ1 (that targets ciliated cells).

Inducible promoters suitable for controlling expression of the therapeutic product include promoters responsive to exogenous agents (e.g., pharmacological agents) or to physiological cues. These response elements include, but are not limited to a hypoxia response element (HRE) that binds HIF-1α and β, a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., pp 167-220, 1991).

In one embodiment, expression of the shRNA and/or other gene product is controlled by a regulatable promoter that provides tight control over the transcription of the gene encoding the shRNA, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that may be used in certain embodiments include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In certain embodiments, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, Mass.).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the shRNA and/or other product can be controlled, for example, by the Tet-on/off system (Gossen et al., 1995, Science 268:1766-9; Gossen et al., 1992, Proc. Natl. Acad. Sci. USA 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA 91(17):8180-4; Schillinger et al., 2005, Proc. Natl. Acad. Sci. USA 102(39):13789-94); the humanized tamoxifen-dep regulatable system (Roscilli et al., 2002, Mol. Ther. 6(5):653-63). The gene switch may be based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT™ system. In one embodiment, the molecule is rapamycin [e.g., marketed as Rapamune™ by Pfizer]. In another embodiment, a rapalog known as AP21967 [ARIAD] is used. Examples of these dimerizer molecules that can be used include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck].

Other suitable enhancers include those that are appropriate for a desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. Other suitable introns include those known in the art, e.g., such as are described in WO 2011/126808. In some embodiments, an intron is a chimeric intron. Examples of suitable polyA sequences include, e.g., rabbit binding globulin (rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence [see, e.g., MA Zanta-Boussif, et al, Gene Therapy (2009) 16: 605-619].

In some embodiments, expression control sequences include sequences that stabilize cytoplasmic mRNA, for example Woodchuck Hepatitis Virus (WHP) Posttranscriptional Regulatory Element (WPRE).

In certain embodiments, the shRNA (or other RNA or transgene) sequences are engineered in a non-viral vector. Such a non-viral vector may be a plasmid carrying an expression cassette which includes, at a minimum, the shRNA coding sequence and optionally, a promoter (e.g., a cardiac troponin T (cTNT) promoter sequence) or other regulatory elements, which is delivered to the heart. Non-viral delivery of nucleic acid molecules to smooth and cardiac muscle systems may include chemical or physical methods. Chemical methods include the use of cationic liposomes (“lipoplex”), polymers (“polyplex”), combinations of the two (“lipopolyplex”), calcium phosphate, and DEAE dextran. Additionally, or optionally, such nucleic acid molecules may be used in a composition further comprising one or more reagents, including, e.g., liposomal reagents such as, e.g., DOTAP/DOPE, Lipofectin, Lipofectamine, etc, and cationic polymers such as PEI, Effectene, and dendrimers. Such reagents are effective for transfecting smooth muscle cells. In addition to the chemical methods, a number of physical methods exist that promote the direct entry of uncomplexed DNA into the cell. These methods can include microinjection of individual cells, hydroporation, electroporation, ultrasound, and biolistic delivery (i.e., the gene gun).

In certain embodiments, an expression cassette comprising at least one shRNA is carried by a viral vector, e.g., a recombinant adenovirus, lentivirus, or adeno-associated virus. In such embodiments, the viral vector is suitably a replication-defective virus. In some embodiments, the expression cassette comprising at least one shRNA is selected from SEQ ID NO: 4, 6, 8, 10, 12, and/or 14.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

### Recombinant AAV

In some embodiments, the RNA targeting the VASH-SVBP complex is expressed from a recombinant adeno-associated virus, which has an AAV capsid and a vector genome packaged in the AAV capsid. In certain embodiments, the AAV capsid is from clade F. See, e.g., [U.S. Pat. No. 7,906,111; WO 2018/160582; WO 2019/168961. For example, in certain embodiments, suitable AAV may include any AAV which effectively targets cardiac cells or permits expression of the, e.g., AAV9, AAVhu31, AAVhu32, AAVhu68, AAV1, AAV2, AAV6, AAV6.2, or another AAV which targets cardiac cells.

In certain embodiments, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Further, the vector genome contains regulatory sequences which direct expression of the gene products. Suitable components of a vector genome are discussed in more detail herein.

In one embodiment, the rAAV is pseudotyped, i.e., the AAV capsid is from a different source AAV than that the AAV which provides the ITRs. In one embodiment, the ITRs of AAV serotype 2 are used. However, ITRs from other suitable sources may be selected. Optionally, the AAV may be a self-complementary AAV.

Where the gene is to be expressed from an AAV, the expression cassettes described herein include an AAV 5′ inverted terminal repeat (ITR) and an AAV 3′ ITR. However, other configurations of these elements may be suitable. In one embodiment, the ITRs are from an AAV different than that supplying a capsid. In one embodiment, the ITR sequences from AAV2. However, ITRs from other AAV sources may be selected. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR is reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable. Where a pseudotyped AAV is to be produced, the ITRs in the expression are selected from a source which differs from the AAV source of the capsid. For example, AAV2 ITRs may be selected for use with an AAV capsid having a particular efficiency for targeting CNS or tissues or cells within the CNS. In one embodiment, the ITR sequences from AAV2, or the deleted version thereof (ΔITR), are used for convenience and to accelerate regulatory approval. However, ITRs from other AAV sources may be selected.

As used herein, an “expression cassette” refers to a nucleic acid molecule which comprises a coding sequence, promoter, and may include other regulatory sequences therefor. In certain embodiments, a vector genome may contain two or more expression cassettes. In other embodiments, the term “transgene” may be used interchangeably with “expression cassette”.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein, “recombinant AAV9 viral particle” refers to nuclease-resistant particle (NRP) which has an AAV9 the capsid having packaged therein a heterologous nucleic acid molecule comprising an expression cassette for a desired gene product. Such an expression cassette typically contains an AAV 5′ and/or 3′ inverted terminal repeat sequence flanking a gene sequence, in which the gene sequence is operably linked to expression control sequences. These and other suitable elements of the expression cassette are described in more detail below and may alternatively be referred to herein as the transgene genomic sequences. This may also be referred to as a “full” AAV capsid. Such a rAAV viral particle is termed “pharmacologically active” when it delivers the transgene to a host cell which is capable of expressing the desired gene product carried by the expression cassette.

In many instances, rAAV particles are referred to as “DNase resistant.” However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a transgene to a host cell and protects these packaged genomic sequences from degradation (digestion) during nuclease incubation steps designed to remove contaminating nucleic acids which may be present from the production process.

As used herein, an “AAV9 capsid” is a self-assembled AAV capsid composed of multiple AAV9 vp proteins. The AAV9 vp proteins are typically expressed as alternative splice variants encoded by a nucleic acid sequence of GenBank accession: AY530579 (SEQ ID NO: 29) which encodes the vp1 amino acid sequence of GenBank accession: AAS99264 (SEQ ID NO: 31). These splice variants result in proteins of different length. In certain embodiments, “AAV9 capsid” includes an AAV having an amino acid sequence which is 99% identical to AAS99264 or 99% identical thereto. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809. See, also, AAV9 deamidation pattern and compositions as described, e.g., in WO 2019/168961. Optionally, AAV capsid may include, e.g., natural isolates (e.g., hu31 or hu32), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in U.S. Pat. Nos. 9,102,949, 8,927,514, US2015/349911; and WO 2016/049230A1. However, in other embodiments, other variants of AAV9, or AAV9 capsids having at least about 95% identity to the above-referenced sequences may be selected. See, e.g., US Published Patent Application No. 2015/0079038. Methods of generating the capsid, coding sequences therefore, and methods for production of rAAV viral vectors have been described. See, e.g., Gao et al, Proc. Natl. Acad. Sci. U.S.A. 100 (10), 6081-6 (2003) and US 2013/0045186A1.

The AAVhu68 capsid is derived from a novel isolate in Clade F. See, e.g., WO 2018/160582, which is incorporated herein by reference in its entirety.

Other suitable AAV capsids may be selected. See, e.g., WO 2019/168961 and WO 2019/169004, published Sep. 6, 2019, which are incorporated by reference herein in their entirely. See also, e.g., WO 2020/223232 A1 (AAV rh.90), WO 2020/223231 A1 (AAV rh.91), and WO 2020/223236 A1 (AAV rh.92, AAV rh.93, AAV rh.91.93), which are incorporated herein by reference in its entirety. These documents also describe other AAV which may be selected for generating AAV and are incorporated by reference. In some embodiments, an AAV capsid (cap) for use in the viral vector can be generated by mutagenesis (i.e., by insertions, deletions, or substitutions) of one of the aforementioned AAV caps or its encoding nucleic acid. In some embodiments, the AAV capsid is chimeric, comprising domains from two or three or four or more of the aforementioned AAV capsid proteins. In some embodiments, the AAV capsid is a mosaic of vp1, vp2, and vp3 monomers from two or three different AAVs or recombinant AAVs. In some embodiments, an rAAV composition comprises more than one of the aforementioned caps.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Methods of preparing AAV-based vectors are known. See, e.g., US Published Patent Application No. 2007/0036760 (Feb. 15, 2007), which is incorporated by reference herein. The use of AAV capsids of AAV9 are particularly well suited for the compositions and methods described herein. The sequences of AAV9 and methods of generating vectors based on the AAV9 capsid are described in U.S. Pat. No. 7,906,111; US2015/0315612; WO 2012/112832; which are incorporated herein by reference. However, other AAV capsids may be selected or generated. For example, the sequences of AAV1, AAV5, and AAV6 are known as are methods of generating vectors. See, e.g., U.S. Pat. No. 7,282,199 B2, U.S. Pat. Nos. 7,790,449, and 8,318,480, which are incorporated herein by reference. The sequences of a number of such AAV are provided in the above-cited U.S. Pat. No. 7,282,199 B2, U.S. Pat. Nos. 7,790,449, 8,318,480, and 7,906,111, and/or are available from GenBank. The sequences of any of the AAV capsids can be readily generated synthetically or using a variety of molecular biology and genetic engineering techniques. Suitable production techniques are well known to those of skill in the art. See, e.g., Sambrook et al, Molecular Cloning: A Laboratory Manual, Cold Spring Harbor Press (Cold Spring Harbor, N.Y.). Alternatively, oligonucleotides encoding peptides (e.g., CDRs) or the peptides themselves can generated synthetically, e.g., by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These and other suitable production methods are within the knowledge of those of skill in the art and are not a limitation.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt) /mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

### Replication-Defective Adenovirus Vectors

In one embodiment, replication-defective adenoviral vectors are used. Any of a number of suitable adenoviruses may be used as a source of the adenoviral capsid sequence and/or in production. See, e.g., U.S. Pat. Nos. 9,617,561; 9,592,284; 9,133,483; 8,846,031; 8,603,459; 8,394,386; 8,105,574; 7,838,277; 7,344,872; 8,387,368; 6,365,394; 6,287,571; 6,281,010; 6,270,996; 6,261,551; 6,251,677; 6,203,975; 6,083,716; 6,019,978; 6,001,557; 5,872,154; 5,871,982; 5,856,152; 5,698,202. Still other adenoviruses are available from the American Type Culture Collection. In one embodiment, the adenoviral particles are rendered replication-defective by deletions in the E1a and/or E1b genes. Alternatively, the adenoviruses are rendered replication-defective by another means, optionally while retaining the E1a and/or E1b genes. The adenoviral vectors can also contain other mutations to the adenoviral genome, e.g., temperature-sensitive mutations or deletions in other genes. In other embodiments, it is desirable to retain an intact E1a and/or E1b region in the adenoviral vectors. Such an intact E1 region may be located in its native location in the adenoviral genome or placed in the site of a deletion in the native adenoviral genome (e.g., in the E3 region).

In the construction of useful adenovirus vectors for delivery of a gene to the human (or other mammalian) cell, a range of adenovirus nucleic acid sequences can be employed in the vectors. For example, all or a portion of the adenovirus delayed early gene E3 may be eliminated from the adenovirus sequence which forms a part of the recombinant virus. The function of E3 is believed to be irrelevant to the function and production of the recombinant virus particle. Adenovirus vectors may also be constructed having a deletion of at least the ORF6 region of the E4 gene, and more desirably because of the redundancy in the function of this region, the entire E4 region. Still another adenoviral vector contains a deletion in the delayed early gene E2a. Deletions may also be made in any of the late genes L1 through L5 of the adenovirus genome. Similarly, deletions in the intermediate genes IX and IVa₂ may be useful for some purposes. Other deletions may be made in the other structural or non-structural adenovirus genes. The above discussed deletions may be used individually, i.e., an adenovirus sequence for use as described herein may contain deletions in only a single region. Alternatively, deletions of entire genes or portions thereof effective to destroy their biological activity may be used in any combination. For example, in one exemplary vector, the adenovirus sequence may have deletions of the E1 genes and the E4 gene, or of the E1, E2a and E3 genes, or of the E1 and E3 genes, or of E1, E2a and E4 genes, with or without deletion of E3, and so on. As discussed above, such deletions may be used in combination with other mutations, such as temperature-sensitive mutations, to achieve a desired result.

An adenoviral vector lacking any essential adenoviral sequences (e.g., E1a, E1b, E2a, E2b, E4 ORF6, L1, L2, L3, L4 and L5) may be cultured in the presence of the missing adenoviral gene products which are required for viral infectivity and propagation of an adenoviral particle. These helper functions may be provided by culturing the adenoviral vector in the presence of one or more helper constructs (e.g., a plasmid or virus) or a packaging host cell. See, for example, the techniques described for preparation of a “minimal” human Ad vector in International Patent Application WO96/13597, published May 9, 1996, and incorporated herein by reference.

Depending upon the adenovirus gene content of the viral vectors employed to carry the expression cassette, a helper adenovirus or non-replicating virus fragment may be necessary to provide sufficient adenovirus gene sequences necessary to produce an infective recombinant viral particle containing the expression cassette. Useful helper viruses contain selected adenovirus gene sequences not present in the adenovirus vector construct and/or not expressed by the packaging cell line in which the vector is transfected. In one embodiment, the helper virus is replication-defective and contains a variety of adenovirus genes in addition to the sequences described above. Such a helper virus is desirably used in combination with an E1-expressing cell line. Helper viruses may also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus may optionally contain a second reporter minigene. A number of such reporter genes are known to the art. The presence of a reporter gene on the helper virus which is different from the transgene on the adenovirus vector allows both the Ad vector and the helper virus to be independently monitored. This second reporter is used to enable separation between the resulting recombinant virus and the helper virus upon purification.

To generate recombinant adenoviruses (Ad) deleted in any of the genes described above, the function of the deleted gene region, if essential to the replication and infectivity of the virus, must be supplied to the recombinant virus by a helper virus or cell line, i.e., a complementation or packaging cell line. In many circumstances, a cell line expressing the human E1 can be used to transcomplement the Ad vector. However, in certain circumstances, it will be desirable to utilize a cell line which expresses the E1 gene products can be utilized for production of an E1-deleted adenovirus. Such cell lines have been described. See, e.g., U.S. Pat. No. 6,083,716. If desired, one may utilize the sequences provided herein to generate a packaging cell or cell line that expresses, at a minimum, the adenovirus E1 gene under the transcriptional control of a promoter for expression in a selected parent cell line. Inducible or constitutive promoters may be employed for this purpose. Examples of such promoters are described in detail elsewhere in this specification. A parent cell is selected for the generation of a novel cell line expressing any desired adenovirus gene. Without limitation, such a parent cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75] cells, among others. These cell lines are all available from the American Type Culture Collection, 10801 University Boulevard, Manassas, Va. 20110-2209. Other suitable parent cell lines may be obtained from other sources. Such E1-expressing cell lines are useful in the generation of recombinant adenovirus E1 deleted vectors. Additionally, or alternatively, cell lines that express one or more adenoviral gene products, e.g., E1a, E1b, E2a, and/or E4 ORF6, can be constructed using essentially the same procedures are used in the generation of recombinant viral vectors. Such cell lines can be utilized to transcomplement adenovirus vectors deleted in the essential genes that encode those products, or to provide helper functions necessary for packaging of a helper-dependent virus (e.g., adeno-associated virus). The preparation of a host cell involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus genomes, combined with polymerase chain reaction, synthetic methods, and any other suitable methods which provide the desired nucleotide sequence.

In still another alternative, the essential adenoviral gene products are provided in trans by the adenoviral vector and/or helper virus. In such an instance, a suitable host cell can be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T 1/2, HEK 293 cells or PERC6 (both of which express functional adenoviral E1) [Fallaux, F J et al, (1998), Hum Gene Ther, 9:1909-17], Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation; nor is the type of mammalian cell, i.e., fibroblast, hepatocyte, tumor cell, etc.

Generally, when delivering the vector comprising the minigene by transfection, the vector is delivered in an amount from about 5 μg to about 100 μg DNA, and preferably about 10 to about 50 μg DNA to about 1×10⁴ cells to about 1×10¹³ cells, and preferably about 10⁵ cells. However, the relative amounts of vector DNA to host cells may be adjusted, taking into consideration such factors as the selected vector, the delivery method and the host cells selected. The vector may be any vector known in the art or disclosed above, including naked DNA, a plasmid, phage, transposon, cosmids, episomes, viruses, etc. Introduction into the host cell of the vector may be achieved by any means known in the art or as disclosed above, including transfection, and infection. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously-added factors, for example. Introduction of the molecules (as plasmids or viruses) into the host cell may also be accomplished using techniques known to the skilled artisan and as discussed throughout the specification. In preferred embodiment, standard transfection techniques are used, e.g., CaPO₄ transfection or electroporation. Assembly of the selected DNA sequences of the adenovirus (as well as the transgene and other vector elements into various intermediate plasmids, and the use of the plasmids and vectors to produce a recombinant viral particle are all achieved using conventional techniques. Such techniques include conventional cloning techniques of cDNA such as those described in texts [Sambrook et al, cited above], use of overlapping oligonucleotide sequences of the adenovirus genomes, polymerase chain reaction, and any suitable method which provides the desired nucleotide sequence. Standard transfection and co-transfection techniques are employed, e.g., CaPO₄ precipitation techniques. Other conventional methods employed include homologous recombination of the viral genomes, plaquing of viruses in agar overlay, methods of measuring signal generation, and the like.

Dosages of the viral vector will depend primarily on factors such as the condition being treated, the age, weight and health of the patient, and may thus vary among patients. For example, a therapeutically effective adult human or veterinary dosage of the viral vector is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for an oral formulation. One of skill in the art may adjust these doses, depending upon the route of administration, and the therapeutic or vaccinal application for which the recombinant vector is employed. The levels of expression of the transgene, or for an immunogen, the level of circulating antibody, can be monitored to determine the frequency of dosage administration. Yet other methods for determining the timing of frequency of administration will be readily apparent to one of skill in the art.

### Lentivirus Systems

A variety of different lentivirus systems are known in the art. See, e.g., WO2001089580 A1 for a method for obtaining stable cardiovascular transduction with a lentivirus system. See, e.g., U.S. Pat. No. 6,521,457. See, also, discussion in N B Wasala, et al, “The evolution of heart gene delivery vectors”, J Gen Med., 2011 October; 13(10): 557-565, which is incorporated herein by reference.

The vectors described herein are suitable formulated into a composition which can be delivered to the patient.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

“Pharmaceutically acceptable excipient” and “pharmaceutically acceptable carrier” refer to a substance or substances that aid the administration of an active agent to and absorption by a subject and can be included in the compositions described herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution alcohols, oils, gelatins, carbohydrates such as lactose, amylase or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the active compounds. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like. One of skill in the art will recognize that other pharmaceutical excipients are useful.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney), oral, inhalation, intranasal, intrathecal, intratracheal, intraarterial, intraocular, intravenous, intramuscular, subcutaneous, intradermal, and other parental routes of administration. Routes of administration may be combined, if desired.

Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector. The dosage will be adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method.

In one embodiment, the viral constructs may be delivered in doses of from at least about least 1×10⁹ GCs to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. However, other pHs within the broadest ranges and these subranges may be selected for other route of delivery.

The term “preparation” is intended to include the formulation of the active moiety. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sub lingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., biologic or viral vector).

In certain embodiments, the composition is specifically targeted (e.g., via direct injection) to the heart. In certain embodiments, the composition or gene of interest is specifically expressed in the heart (e.g., cardiomyocytes).

In certain embodiments, the pharmaceutical composition comprising a replication-defective virus as described herein is administered to a patient. In certain embodiments, the patent has heart failure associated with failing cardiomyoctes. In certain embodiments, the patient has heart failure with reduced ejection fraction (HFrEF). In certain embodiments, the patient has heart failure with preserved ejection fraction (HFpEF). In certain embodiments, the pharmaceutical composition is used in reducing viscoelasticity and/or speed up cardiomyocyte relaxation. In some embodiments, the pharmaceutical composition is used in increasing the speed of cardiomyocyte relaxation via a Ca²⁺-independent mechanism. In certain embodiments, the pharmaceutical composition is used for reducing cardiomyocyte stiffness, improving myocyte relaxation, increasing ventricular compliance, and/or reducing diastolic pressures. In certain embodiments, the pharmaceutical composition is used in treating patients with diastolic dysfunction. In certain embodiments, the pharmaceutical composition is used in preparing a medicament for use in treatment of failing cardiomyocytes. In certain embodiments, the pharmaceutical composition is used in preparing a medicament for use in improvement of kinetics of failing ventricular cardiomyocytes. In certain embodiments, the pharmaceutical composition is used in preparing a medicament for use in reducing viscoelasticity of cardiomyocytes. In certain embodiments, the pharmaceutical composition comprises a replication-defective vector comprising at least one shRNA as described herein. In certain embodiments, the pharmaceutical composition comprises the vector, wherein the vector comprises two or more shRNA. In certain embodiments, the pharmaceutical composition comprising the vector comprising at least one shRNA, and optionally with a second therapeutic transgene (e.g., ttl).

In certain embodiments, the method for treating failing human cardiomyocytes comprising delivering a replication-defective vector or a composition as described wherein to the patient as a sole therapeutic intervention. Such a vector may express at least one shRNA as described herein. In certain embodiments, the vector expresses two or more shRNA. In certain embodiments, the vector expresses at least one shRNA, optionally with a second therapeutic transgene (e.g., ttl). In certain embodiments, the vector is co-administered with in a combination therapy with one or more of a calcium desensitizer, myosin inhibitors, a small molecule VASH inhibitor, a small molecule small vasohibin binding protein inhibitor, tubulin tyrosine ligase, and/or parthenolide therapy.

In certain embodiments, the compositions provided herein here may be co-administered with a tubulin-tyrosine ligase (TTL). Optionally, the viral vectors may be engineered to express one or more shRNA and a TTL from the same vector. In other embodiments, compositions may comprise a vector expressing one or more shRNA targeted to VASH and a second vector which expresses TTL. In still other embodiments, TTL may be delivered as a protein. In certain embodiments, the nucleic acid molecule is specifically targeted to the heart. In still other embodiments, the nucleic acid molecule is specifically targeted to the cardiac microtubules. The nucleic acid may be delivered by non-viral delivery systems and/or by viral delivery systems. Optionally, the therapy may involve co-administration of two or more of the enzyme, a nucleic acid expressing the enzyme, and/or a small molecule drug which reduces detyrosination and/or inflammation.

As used herein, the term “tubulin-tyrosine ligase” refers to a human enzyme which catalyzes the post-translational addition of a tyrosine to the C-terminal end of detyrosinated alpha-tubulin. One suitable human amino acid sequence is provided in UNIPROT [Q8NG68] (377 amino acids in length), available at: www.uniprot.org/uniprot/Q8NG68:

(SEQ ID NO: 27)
MYTFVVRDENSSVYAEVSRLLLATGHWKRLRRDNPRFNLMLGERNRLPF
GRLGHEPGLVQLVNYYRGADKLCRKASLVKLIKTSPELAESCTWFPESY
VIYPTNLKTPVAPAQNGIQPPISNSRTDEREFFLASYNRKKEDGEGNVW
IAKSSAGAKGEGILISSEASELLDFIDNQGQVHVIQKYLEHPLLLEPGH
RKFDIRSWVLVDHQYNIYLYREGVLRTASEPYHVDNFQDKTCHLTNHCI
QKEYSKNYGKYEEGNEMFFKEFNQYLTSALNITLESSILLQIKHIIRNC
LLSVEPAISTKHLPYQSFQLFGFDFMVDEELKVWLIEVNGAPACAQKLY
AELCQGIVDIAISSVFPPPDVEQPQTQPAAFIKL .

Any suitable coding sequence for this protein may be back translated, optionally taking into consideration the codons preferred for human use. Such a nucleic acid sequence may be DNA (e.g., cDNA) or RNA (e.g., mRNA, tRNA, among others).

In one embodiment, a defective vector is provided which comprises a nucleic acid sequence encoding tubulin tyrosine ligase (TTL) under the control of a regulatory control sequence which directs expression thereof in the heart. See, e.g., US Published Patent Application No. US 2018/0326022, published Nov. 15, 2018, which is incorporated by reference in its entirety.

In one aspect, a method for improving heart function in humans is provided for treating a patient in a combination regimen with a further therapeutic which inhibits tubulin carboxypeptidase (TCP). In certain embodiments, the therapeutic (active ingredient) may be sesquiterpene lactones, such as parthenolide or costunolide, or a prodrug, derivative, pharmaceutically acceptable salt or analog thereof. In yet another embodiment, the therapeutic is an inhibitor of TCP activity such as epoY, epoEY, or epoEEY.

In another aspect, a method for treating heart failure in humans is provided which comprises dosing a patient with a co-therapeutic which interferes with detyrosinated microtubules in cardiomyocytes. The therapeutic may be a small molecule drug selected from one or more of: sesquiterpene lactones including parthenolide (PTL), costunolide or PTL pro-drugs such as LC-1, or microtubule destabilizers including colchicine, vinblastine, and nocodazole. In certain embodiments, a method is provided for treating patients with a composition which decreases detyrosination of cardiac microtubules. This method is useful for stabilizing loss of heart function and/or preventing heart failure in patients (e.g., humans). The therapeutic may be a small molecule drug selected from one or more of: sesquiterpene lactones including parthenolide (PTL), costunolide or PTL pro-drugs such as LC-1, or microtubule destabilizers including colchicine, vinblastine, and nocodazole. In yet another embodiment, the therapeutic is an inhibitor of TCP activity such as epoY, epoEY, or epoEEY. In certain embodiments, a method is provided for treating patients with a composition which decreases or prevents detyrosination of cardiac microtubules.

In certain embodiments, epoY, epoEY, or epoEEY, may be selected for use in a combination therapy described herein. EpoY, epoEY, and epoEEY contain the epoxide functional group from parthenolide coupled to one, two, or three amino acids from the α-tubulin C terminus, respectively. (Single-letter abbreviations for the amino acid residues are as follows: A, Ala; C, Cys; D, Asp; E, Glu; F, Phe; G, Gly; H, His; I, Ile; K, Lys; L, Leu; M, Met; N, Asn; P, Pro; Q, Gln; R, Arg; S, Ser; T, Thr; V, Val; W, Trp; and Y, Tyr. See, e.g., Aillaud et al, Science 358 (6369), 1448-1453 and the supplementary materials therewith, incorporated by reference in its entirety, for an illustrative description of synthesis of these compounds. See, e.g., US Published Patent Application No. US 2018/0326022, published Nov. 15, 2018, which is incorporated by reference in its entirety.

The term “pharmaceutically acceptable salts” includes salts of the active compounds that are prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When the compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolyl-sulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66: 1-19 (1977)). Certain specific compounds contain both basic and acidic functionalities that allow the compounds to be converted into either base or acid addition salts. Other pharmaceutically acceptable carriers known to those of skill in the art are suitable. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Methods for preferentially targeting cardiac cells and/or for minimizing off-target non-cardiac gene transfer have been described.

In certain embodiments, a method such as that in U.S. Pat. No. 7,399,750, is used to increase the dwell time of the vector carrying the gene of interest in the heart by the induction of hypothermia, isolation of the heart from circulation, and near or complete cardiac arrest. Permeabilizing agents are an essential component of this method and are used during the administration of the virus to increase the uptake of the virus by the cardiac cells. This method is particularly well suited to viral vectors, where the gene expression may be is highly specific to cardiac muscle and, in particularly in the case of rAAV vectors, expression may be maintained long-term, with no signs of myocardiac inflammation. Still other systems and techniques may be used including, without limitation, e.g., a “bio-pacemaker”, such as that described in U.S. Pat. No. 8,642,747, US-2011-0112510.

Where co-administered separately from a vector containing a VASH1-SVBP inhibitor, one or more of the compounds identified herein may be administered alone or can be co-administered further in a combination with one or more active compounds to the patient. These compositions can be delivered transdermally, by a topical route, formulated as applicator sticks, solutions, suspensions, emulsions, gels, creams, ointments, pastes, jellies, paints, powders, and aerosols. Oral preparations include tablets, pills, powder, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions may additionally include components to provide sustained release and/or comfort. Such components include high molecular weight, anionic mucomimetic polymers, gelling polysaccharides and finely-divided drug carrier substrates. The compositions can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1 995; as biodegradable and injectable gel formulations (see, e.g., Gao Pharm. Res. 12: 857-863, 1 995); or, as micro spheres for oral administration (see, e.g., Eyles, J. Pharm. Pharmacol. 49: 669-674, 1997). In another embodiment, the compositions can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ (e.g., the heart), one can focus the delivery of the compositions into the target cells in vivo.

Co-administration is meant to include simultaneous or sequential administration of the compound individually or in combination (more than one compound or agent) with the vector containing the VASH-SVBP complex inhibitor. Thus, the preparations can also be combined, when desired, with other active substances.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

Identity or similarity with respect to a sequence is defined herein as the percentage of amino acid residues in the candidate sequence that are identical (i.e., same residue) or similar (i.e., amino acid residue from the same group based on common side-chain properties, see below) with the peptide and polypeptide regions provided herein, after aligning the sequences and introducing gaps, if necessary, to achieve the maximum percent sequence identity. Percent (%) identity is a measure of the relationship between two polynucleotides or two polypeptides, as determined by comparing their nucleotide or amino acid sequences, respectively. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology. There are a number of algorithms, and computer programs based thereon, which are available to be used the literature and/or publicly or commercially available for performing alignments and percent identity. The selection of the algorithm or program is not a limitation.

Examples of suitable alignment programs including, e.g., the software CLUSTALW under Unix and then be imported into the Bioedit program (Hall, T. A. 1999, BioEdit: a user-friendly biological sequence alignment editor and analysis program for Windows 95/98/NT. Nucl. Acids. Symp. Ser. 41:95-98); the Clustal Omega available from EMBL-EBI (Sievers, Fabian, et al. “Fast, scalable generation of high-quality protein multiple sequence alignments using Clustal Omega.” Molecular systems biology 7.1 (2011): 539 and Goujon, Mickael, et al. “A new bioinformatics analysis tools framework at EMBL-EBI.” Nucleic acids research 38.suppl 2 (2010): W695-W699); the Wisconsin Sequence Analysis Package, version 9.1 (Devereux J. et al., Nucleic Acids Res., 12:387-395, 1984, available from Genetics Computer Group, Madison, Wis., USA). The programs BESTFIT and GAP, may be used to determine the % identity between two polynucleotides and the % identity between two polypeptide sequences.

Other programs for determining identity and/or similarity between sequences include, e.g., the BLAST family of programs available from the National Center for Biotechnology Information (NCB), Bethesda, Md., USA and accessible through the home page of the NCBI at www.ncbi.nlm.nih.gov), the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used; and FASTA (Pearson W. R. and Lipman D. J., Proc. Natl. Acad. Sci. USA, 85:2444-8, 1988, available as part of the Wisconsin Sequence Analysis Package). SeqWeb Software (a web-based interface to the GCG Wisconsin Package: Gap program).

In certain embodiments, the shRNA or other sequences are designed to be specifically recognized by target sequences in the VASH-SVBP complex.

As used herein, the term “target cell” refers to any target cell in which expression of the functional gene product (e.g., shRNA) is desired. Examples of target cells may include, but are not limited to, cardiac cells and cardiomyoctes.

The term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “about” encompasses a variation within and including ±10%, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### Example 1—VASH1- and VASH2-SVBP Complexes Act as a Tubulin Carboxypeptidase in Cardiomyocytes with VASH1 being Predominant

Heart failure is often characterized by diastolic dysfunction, or inadequate ventricular filling due to the inability of the myocardium to fully relax. Diastolic dysfunction is a hallmark of hypertrophic cardiomyopathy (HCM)1 and heart failure with preserved ejection fraction (HFpEF), (HFrEF) diseases often characterized by prolonged and impaired left ventricular (LV) relaxation, slow LV filling, and increased diastolic LV stiffness.2 Diastolic dysfunction can be initiated by factors extrinsic to the cardiomyocyte such as hypertension and fibrosis, or intrinsic impairments to cardiomyocyte calcium cycling and myofilament calcium sensitivity.

In addition to mechanisms that directly control sarcomere contraction and relaxation, myocyte motion can be modulated by the mechanical properties of the non-sarcomeric cytoskeleton.

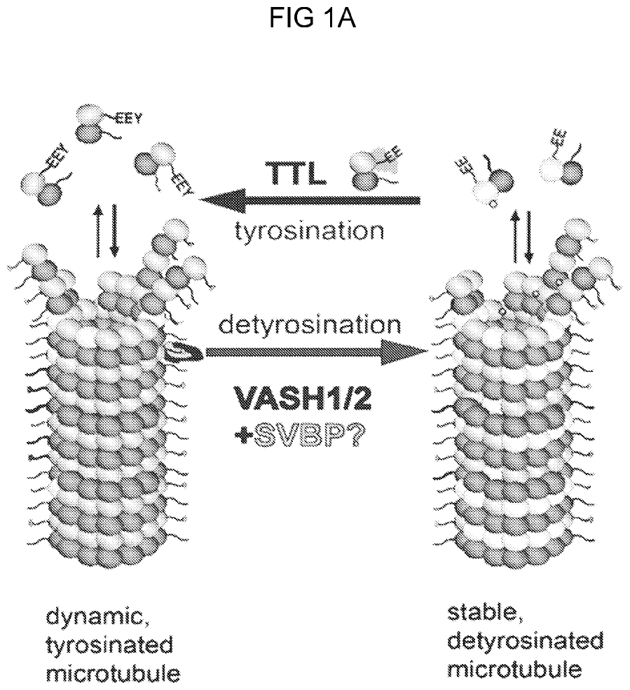

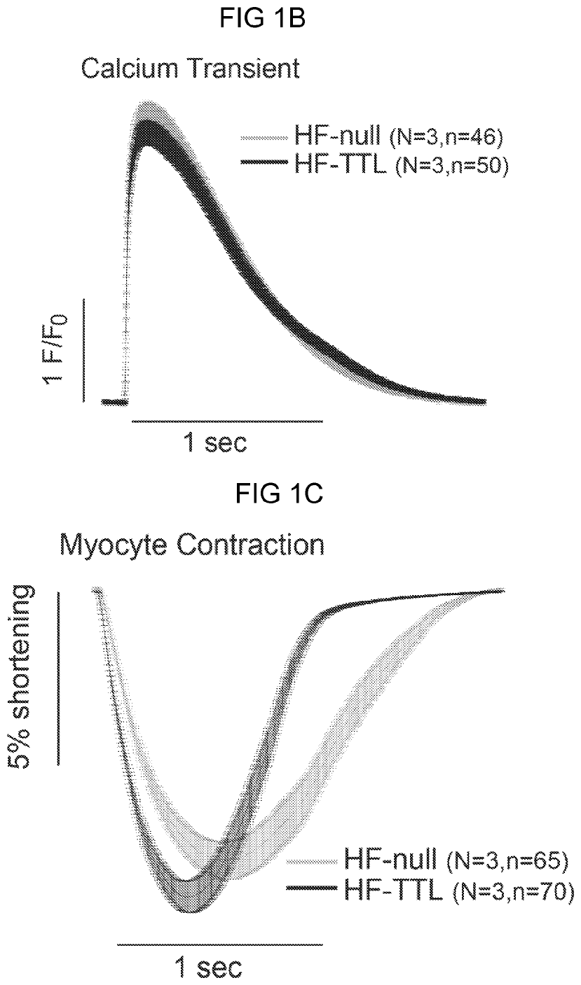

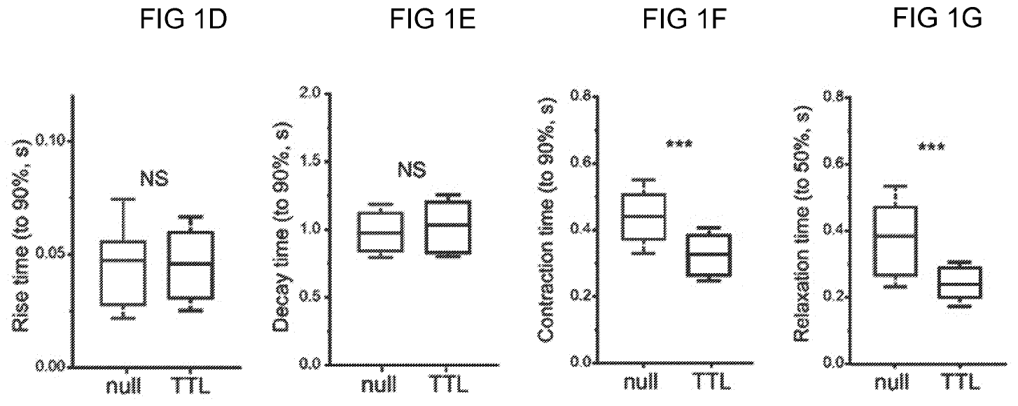

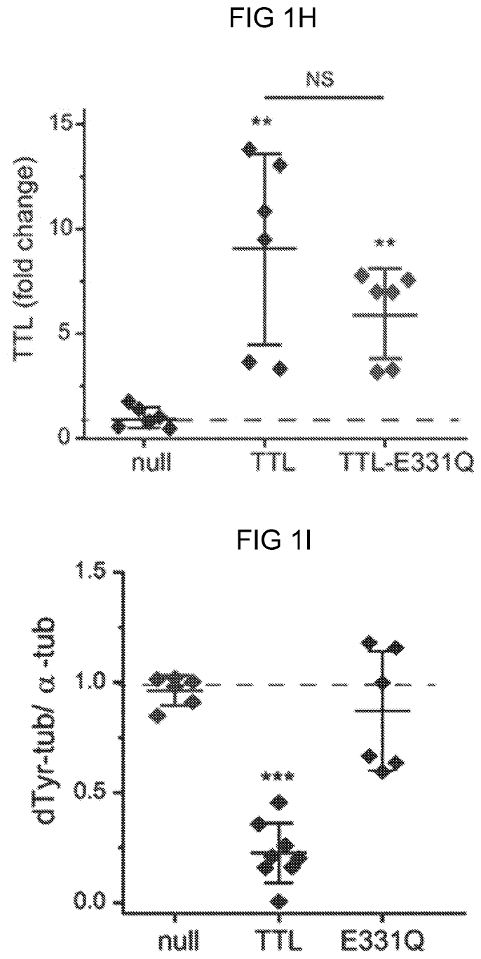

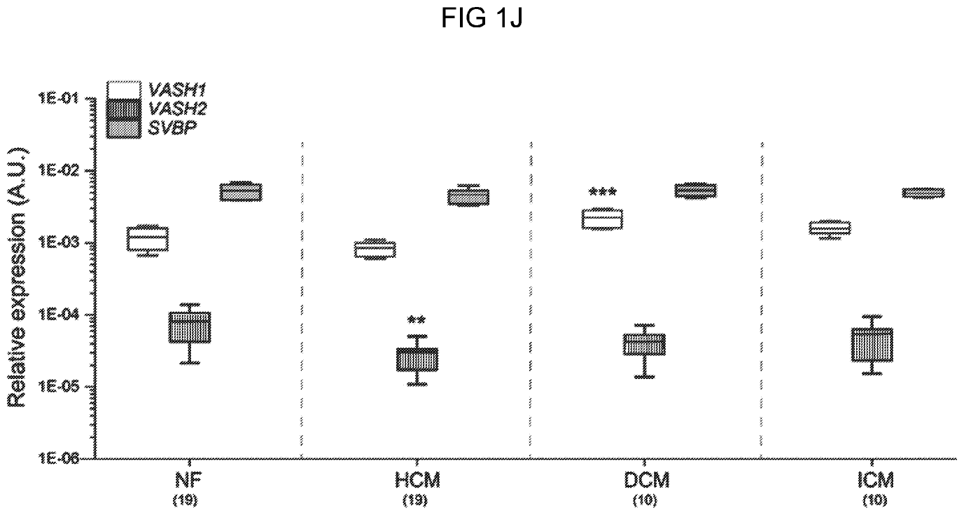

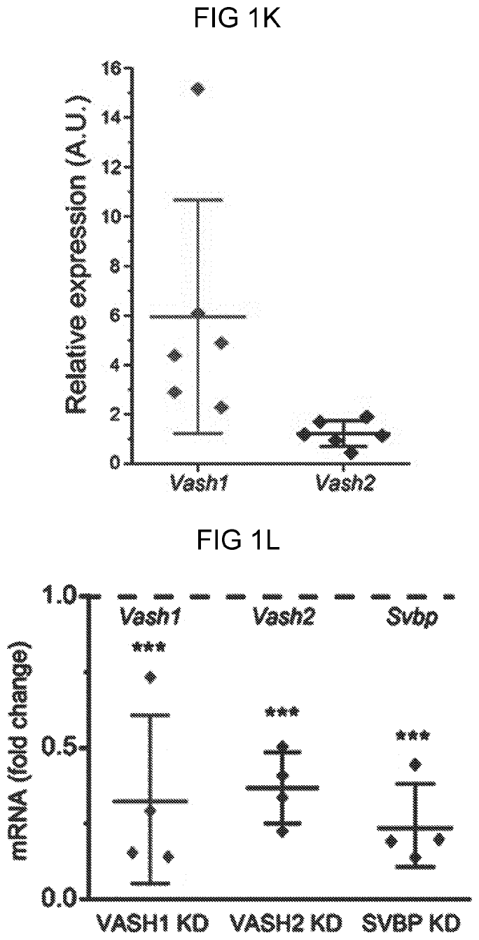

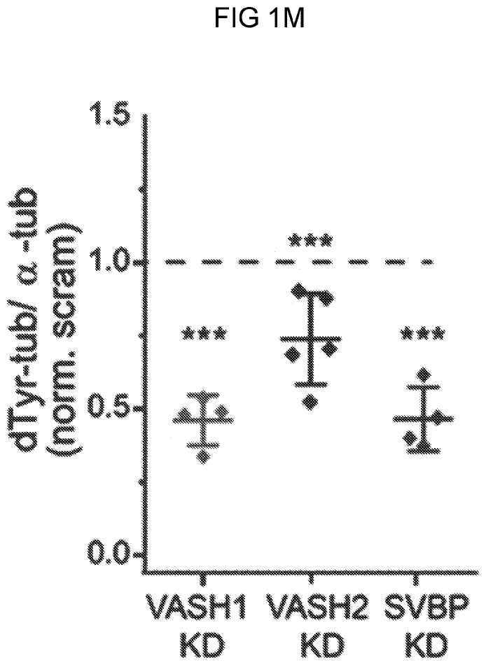

Cardiac microtubules provide viscoelastic resistance to myofilament shortening and re-lengthening through physical coupling of microtubules to the myofilaments.^{3,4} This physical interaction is tuned by detyrosination, a post-translational modification of the C-terminal tail of α-tubulin (FIG. 1A). In human heart failure (HF), the cardiac microtubule network proliferates and becomes more detyrosinated, increasing viscous drag on the myofilaments.⁵ Multiple recent proteomic and biochemical assessments of patient myocardium suggest that detyrosinated microtubules are upregulated across the spectrum of end-stage HF,⁵ as well as in patients with aortic stenosis⁶ or inherited HCM.⁷ Together, this data implicates microtubule detyrosination as a therapeutic target to lower stiffness and improve myocyte motion in HF.

The recent identification of a detyrosinating enzyme complex of vasohibin-1 or -2 (VASH1/2) and their obligate partner small vasohibin binding protein (SVBP) potentially enables the development of more specific approaches to reduce detyrosination.^{11,12} However, alternative tubulin carboxypeptidases beyond VASH exist in other cell types,¹² and it remains to be determined if either VASH1 or VASH2 are active detyrosinases in cardiomyocytes, and whether their selective inhibition is sufficient to improve contractility. In this study, we identify the VASH1-SVBP complex as a predominant tubulin carboxypeptidase in cardiomyocytes.

### Materials and Methods

### Human Myocardial Tissue

Procurement of human myocardial tissue was performed under protocols and ethical regulations approved by Institutional Review Boards at the University of Pennsylvania and the Gift-of-Life Donor Program (Pennsylvania, USA) and as described.⁵ Briefly, failing human hearts of non-ischemic origin were procured at the time of orthotropic heart transplantation at the Hospital of the University of Pennsylvania following informed consent from all participants. Non-failing (NF) hearts were obtained at the time of organ donation from cadaveric donors. In all cases, hearts were arrested in situ using ice-cold cardioplegia solution and transported on wet ice. Whole hearts and dissected left ventricle were weighed to determine levels of hypertrophy. Failing hearts are etiologically defined by clinical diagnosis of HF, which is subdivided into HFpEF (ejection fraction >50%) and HFrEF (ejection fraction <30%). Hearts were utilized for a particular experiment as they arrived until the required sample size was reached for each etiology of interest (e.g., triplicate quality isolations and experiments for NF, HFpEF and HFrEF).

For this study myocytes were isolated from 22 hearts (see method details below) for functional studies. For further details on classification, descriptive statistics, and experiments performed on each heart, see e.g., Chen et al., 2020 Circulation Research, 127(2):e14-e27, Table 1 Supplementary Materials.

Human Left Ventricular Myocyte Isolation and Cell Culture Human left ventricular myocytes were isolated as described previously.⁵ Culture medium consisted of F-10 (1×) Nutrient Mixture (Ham) [+] L-Glutamine (Life Technologies, 11550-043) supplemented with insulin-transferrin selenium-X (Gibco, 51500-056), 20 mmol/L HEPES, 1 μg μl⁻¹ primocin (Invivogen, ant-pm-1), 0.4 mmol/L extra CaCl₂), 5% FBS, and 25 μmol/L cytochalasin D (Cayman, 11330). Viable myocytes were concentrated by gravity and the proper amount of medium was added in culture so that neighboring cells were not in direct contact. Viral constructs were permitted to express for 48 hours with a multiplicity of infection=100-200.

### Animals

Animal care and procedures were approved and performed in accordance with the standards set forth by the University of Pennsylvania Institutional Animal Care and Use Committee and the Guide for the Care and Use of Laboratory Animals published by the US National Institutes of Health.

### Rat Cardiomyocyte Isolation and Culture

Primary adult ventricular cardiac myocytes were isolated from 6 to 8 week old Sprague Dawley rats as previously described.¹³ Briefly, the heart was removed from a rat anesthetized under isoflurane and retrograde-perfused on a Langendorff apparatus with a collagenase solution. The digested heart was then minced and triturated using a glass pipette. The resulting supernatant was separated and centrifuged at 300 revolution per minute (rpm) to isolate cardiomyocytes which were then resuspended in rat cardiomyocyte media at low density. Cardiomyocytes were cultured at 37° C. and 5% CO₂ with 25 μmol/L of cytochalasin D. The viability of rat cardiomyocytes upon isolation is typically on the order of 50-75% rod-shaped, electrically excitable cells, and the survivability for 48 hrs of culture is >80% (See Heffler et al.¹⁴ for our quantification of cardiomyocyte morphology in culture). While the viability of human cardiomyocyte isolations is lower (with more variation between preparations), the 48 hr survivability is similar to rat cardiomyocytes, and did not differ between experimental groups.

Rat cardiomyocyte media: medium 199 (Thermo Fisher 115090) supplemented with 1× Insulin-transferrin-selenium-X (Gibco 51500056), 1 μg μl⁻¹ primocin (Invivogen ant-pm-1), 20 mmol/L HEPES at pH 7.4 and 25 μmol/L cytochalasin D.

### Contractility and Calcium Transient Measurement

Experiments were performed as previously described.⁵ Briefly, prior to contractility measurement, cultured human myocytes were transferred to fresh warm medium without cytochalasin D. Contractility was measured in a custom-fabricated cell chamber (IonOptix) mounted on an LSM Zeiss 880 inverted confocal microscope using either a 40 or 63× oil 1.4 numerical aperture objective and transmitted light camera (IonOptix MyoCam-S). Experiments were conducted at room temperature and field stimulation was provided at 0.5 Hz with a cell stimulator (MyoPacer, IonOptix). After 10-30 seconds of pacing to achieve steady state, five traces were recorded and analyzed. Sarcomere length was measured optically by Fourier transform analysis (IonWizard, IonOptix).

For simultaneous calcium and contractility measurement, myocytes were incubated with 2 μmol/L fluo-3-acetoxymethyl ester (Invitrogen) on rocker for 10 minutes at room temperature, then enriched by gravity for 5 minutes and replenished with fresh warm medium without cytochalasin D. Sarcomere length and fluo3 fluorescence were measured in a custom-fabricated cell chamber (IonOptix) mounted on a Zeiss inverted microscope using a 40× water 1.2 numerical aperture objective and CCD video camera (MyoCam-S3) coupling to a cell framing adaptor that connects to a photomultiplier tube (PMT400 Sub-System, IonOptix). Cell framing box for fluorescence detection was set at a fixed size. Fluorescence level of a blank area in the dish was recorded to account for background noise. Myocytes were electrically paced at 0.5 Hz at room temperature and both sarcomere length and fluorescence counts were recorded simultaneously for five steady-state transients.

### Generation of Short Hairpin RNAs

Adenoviruses encoding shVASH and shSVBP constructs were generated and produced in a similar manner as previously described³, but directed towards single target sites under the U6 promotor in three separate viruses (SEQ ID NOs: 4, 6, 8, 10, 12, and 14). Target sites for VASH1: sh1: gctgcagtacaatcacacagg (Nucleotide 1-21 of SEQ ID NO: 16), sh2: gggacacagttctttgaaatt (Nucleotide 1-21 of SEQ ID NO: 17), sh3: gggaatttacctcaccaacag (Nucleotide 1-21 of SEQ ID NO: 18); for SVBP: sh1: gacaaagagcagagatctatg (Nucleotide 1-21 of SEQ ID NO: 19), sh2: gcagcagcagtttgatgagtt (Nucleotide 1-21 of SEQ ID NO: 20), sh3: gcagcagtttgatgagttctg (Nucleotide 1-21 of SEQ ID NO: 21); for VASH2: sh1: gtcaagaaggtcaagattggg (Nucleotide 1-21 SEQ ID NO: 24), sh2: ggtcaagattgggctgtacgt (Nucleotide 1-21 SEQ ID NO: 25), sh3: gtcaagattgggctgtacgt (Nucleotide 1-21 SEQ ID NO: 26). eBFP2 was used as a marker of transduction. For data in the primary figures, VASH1 sh1, SVBP sh3, and VASH2 sh2 were utilized.

RNA Isolation, cDNA Synthesis, and RT-qPCR

RNA was isolated from cardiomyocytes using RNAzol RT (Molecular Research Center RN190) following the manufacturer's instructions. Briefly, cardiomyocytes were lysed in RNAzol RT reagent. One ml of the lysate was combined with 0.4 ml of water and shaken vigorously for 15 seconds and stored for 15 minutes at room temperature. Samples were then centrifuged at 12,000 g for 15 minutes. The supernatant was removed to a new tube and mixed with one volume of isopropanol, stored at room temperature for 10 minutes, then centrifuged at 12,000 g for 10 minutes. The RNA pellet was then washed with 75% ethanol two times and solubilized in RNase-free water. RNA concentration was determined using a Nanodrop (ThermoFisher) and 2 g RNA was reverse transcribed using cDNA synthesis kit (TaKaRa #6110A or SuperScript IV Thermo Fisher #18091150) following manufacturer's instructions. Twenty ng of cDNA template was then used to conduct RT-qPCR in three technical replicates. For Vash1 and Svbp knockdown data sets, Powerup SYBR green master mix (#A25742, Thermo Fisher) was used with the following primers: Gapdh (F-5′-CGTGCCGCCTGGAGAAAC-3′ (SEQ ID NO: 32) and R-5′-TGGGAGTTGCTGTTGAAGTCG-3′ (SEQ ID NO: 33)), Vash1 (F-5′-TCGTCGGCTGGAAAGTAGGCAC-3′ (SEQ ID NO: 34) and R-5′-TCGTCGGCTGGAAAGTAGGCAC-3′ (SEQ ID NO: 35)), Svbp (F-5′-AACCAGCCTTCAGAGTGGAGAAGG-3′ (SEQ ID NO: 36) and R-5′-GCTCCGTCATGACTCTGTTGAGAGC-3′ (SEQ ID NO: 37)); for Vash2 knockdown and relative transcript levels of Vash1 and Vash2 in rat myocytes, PrimeTime gene expression master mix (#1055772) with the following primers/probes: Vash2 (probe 5′-/56-FAM/TCAAGATCT/ZEN/TCATCCGCATGTCCCTG/3IABkFQ/-3′ (SEQ ID NO: 38), F-5′-GAAGCAACTTGTCCTCAATGTC-3′ (SEQ ID NO: 39) and R-5′-GGATTCTCACTTGGGTTGGAG-3′ (SEQ ID NO: 40)) (assay name Rn.PT.58.45226291); Vash1 (probe 5′-56-FAM/TGCCTACTT/ZEN/TCCAGCCGACAACG/3IABkFQ/-3′ (SEQ ID NO: 41), F-5′-GCCCAAGATTCCCATACCAA-3′ (SEQ ID NO: 42) and R-5′-ACTGTGTCCCTGTGTGATTG-3′ (SEQ ID NO: 43)) (assay name Rn.PT.58.37363926); Gapdh (probe 5′-/56-FAM/CAGCACCAG/ZEN/CATCACCCCATTTG/3IABkFQ/-3′ (SEQ ID NO: 44), F-5′-AACCCATCACCATCTTCCAG-3′ (SEQ ID NO: 45) and R-5′-CCAGTAGACTCCACGACATAC-3′ (SEQ ID NO: 46)) (assay name Rn.PT.39a.11180736.g) (Integrated DNA Technologies). Cycle threshold (Ct) values were quantified on a QuantStudio 3 Real Time PCR system (ThermoFisher). Gene expression fold change was quantified using the delta-delta Ct method normalized to Gapdh and the scram group.

For RT-qPCR experiments in human samples, total RNA was extracted from septal myectomies of HCM patients (N=19), from explanted hearts of DCM patients (N=10) and from ischemic cardiomyopathy (ICM, N=10) patients as well as from NF human heart tissue not suitable for transplantation or from donors that did not die from cardiac disease but of another cause (NF, N=10-11) using the SV Total RNA Isolation Kit (Promega) according to the manufacturer's instructions and as described previously.¹⁵ RNA concentration and purity was determined photometrically using the Nanodrop ND-1000. RNA was reverse-transcribed to cDNA using the Superscript III (Invitrogen) kit. Subsequently, RT-qPCR was performed using the Maxima SYBR Green/ROX qPCR Master Mix (Thermo Scientific) and the specific following primers: GAPDH (F-5′-ATGTTCGTCATGGGTGTGAA-3′ (SEQ ID NO: 47) and R-5′-TGAGTCCTTCCACGATACCA-3′ (SEQ ID NO: 48)), VASH1 (F-5′-AGAGGAAGGGGAAGAGGACC-3′ (SEQ ID NO: 49) and R-5′-GTAGGCACACTCGGTATGGG-3′ (SEQ ID NO: 50)), VASH2 (F-5′-GTTCCACGTCAACAAGAGCG-3′ (SEQ ID NO: 51) and R-5′-CGACAGCCTGTAGTTTGGGA-3′ (SEQ ID NO: 52)), SVBP (F-5′-CAGCAGAGTTGAGAAGGCCA-3′ (SEQ ID NO: 53) and R-5′-CCAGGAGGCTGCATCTGTTT-3′ (SEQ ID NO: 54)). Relative gene expression of VASH1, VASH2 and SVBP was calculated using the delta Ct method (with a formula 2\hat{ }(−delta Ct)) normalized to GAPDH.

### Microtubule Fractionation Assay

Isolated rat cardiomyocytes were pelleted by gravity, washed with warm microtubule stabilizing buffer containing 0.1 mol/L PIPES (pH 6.8), 2 mmol/L EGTA, 0.1 mmol/L EDTA, 0.5 mmol/L MgCl₂, 20% glycerol, and centrifuged at 300 rpm for 2 minutes. The resulting pellet was resuspended in 150 μl microtubule-stabilizing buffer supplemented with 0.1% Triton X-100 and 1× protease and phosphatase Inhibitor cocktail (Cell Signaling #5872S) and incubated for 30 minutes at 37° C. Next, cells were centrifuged at 300 rpm for 2 minutes and the supernatant was collected as the free tubulin fraction. The resulting pellet was resuspended in 150 μl RIPA buffer (Cayman #10010263) supplemented with extra 0.8% SDS, disrupted by pipetting every 15 minutes on ice for 1 hour, then boiled at 100° C. for 3 minutes. Finally, the fraction was centrifuged at 12,000 g for 2 minutes and the supernatant was collected as the polymerized fraction.

### Western Blot

For whole cell protein extraction, isolated rat cardiomyocytes were lysed in RIPA buffer (Cayman #10010263) supplemented with protease and phosphatase Inhibitor cocktail (Cell Signaling #5872S) on ice for 1 hour. The supernatant was collected and combined with 4× loading dye (Li-COR #928-40004), supplemented with 10% 2-mercaptoethonol, and boiled for 8 minutes. The resulting lysate was resolved on SDS-PAGE gel and protein was blotted to nitrocellulose membrane (Li-COR #926-31902) with mini Trans-Blot Cell (Bio-Rad). Membranes were blocked for an hour in Odyssey Blocking Buffer (TBS) (LI-COR #927-50000) and probed with corresponding primary antibodies overnight at 4° C. Membranes were rinsed with TBS containing 0.5% Tween 20 (TBST) three times and incubated with secondary antibodies TBS supplemented with extra 0.2% Tween 20 for 1 hour at room temperature. Membranes were washed again with TBST (0.5% Tween 20) and imaged on an Odyssey Imager. Image analysis was performed using Image Studio Lite software (LI-COR). All samples were run in duplicates and analyzed in reference to GAPDH.

### Antibodies and Labels

Detyrosinated tubulin; rabbit polyclonal (Abcam ab48389); western blot: 1: 1,000. Alpha tubulin; mouse monoclonal, clone DM1A (Cell Signaling #3873); western blot: 1:1,000

TTL; rabbit polyclonal (Proteintech 13618-1-AP); western blot: 1:500.

GAPDH; mouse monoclonal (VWR GenScript A01622-40); western blot: 1:1,000.

IRDye 800CW Donkey anti-Mouse IgG (H+L) (LI-COR 925-32212); western blot: 1:10,000.

IRDye 680RD Donkey anti-Rabbit IgG (H+L) (LI-COR 925-68073); western blot: 1:10,000.

IRDye 680RD Donkey anti-Mouse IgG (H+L) (LI-COR 926-68072); western blot: 1:10,000.

IRDye 800CW Donkey anti-Rabbit IgG (H+L) (LI-COR 926-32213); western blot: 1:10,000.

VASH1; rabbit polyclonal (Abcam ab199732); western blot 1:5000-1:1000

An antibody for human and mouse VASH1 (named anti-VASH1 Gre) was produced in rabbits by using a peptide C-RIRGATDLPKIPIPSVPTFQPTTPV-NH2 ((SEQ ID NO: 55) corresponding to exposed region of the protein, see Wang, Bose and Choi et al.)¹⁶ linked at the N-terminal to the keyhole limpet hemocyanin protein via the cysteine. Sera from rabbits were purified on the respective peptides. Validation of these antibodies was performed in HEK293T cells protein extracts. HEK293T cultures and transfections, as well as SDS-PAGE and immunoblots, were performed as in Aillaud et al.¹¹

Co-Immunoprecipitation (co-IP)

Isolated rat cardiomyocytes were lysed in IP buffer containing Tris 50 mmol/L pH 8, NaCL 150 mmol/L, NP40 1% and protease inhibitor cocktail (Cell Signaling #5872S) on ice for 30 minutes with pipetting every 10 minutes. Protein concentration was quantified using Braford dye reagent (Bio-Rad #5000205). Equal amount of protein (1.5 mg) from each sample was incubated with 6.5 g of anti-SVBP (Gre) and 10 μl of Dynabeads protein G (Invitrogen 10004D, rinsed four times with ice-cold PBS before used), mixed on a tube revolver (Thermo Fisher #11-676-341F5). Negative controls for co-IP were run with Rabbit IgG (6.5 g) or without antibody. After overnight incubation at 4° C., beads were rinsed with IP buffer for four times and eluted at room temperature using 30 μl 1× Laemli buffer (Bio-Rad) supplemented with 2-mercaptoethonol. Elution supernatant was then transferred to fresh tubes and heated at 70° C. for 5 min. Fifteen μl of the elution from co-IP and whole cells lysates were used in western blot, performed as described above except the followings. Blots were stained with anti-VASH1 (Gre) at 1:1,000 at 4° C. overnight and a secondary antibody against native rabbit IgG at 1:1,000 (TrueBlot anti-rabbit IgG HRP from Rockland #18-8816-31) at room temperature for 1 hour. Blots were developed using a chemiluminescent substrate (West Pico Plus, Thermo Fisher #34577) and autoradiography film (Hyblot CL #E3012).

### Statistics

Statistical analysis and graphing were performed using OrginPro software (OriginLabs). For box plots, box represents 25^{th} to 75^{th} percentiles, the whiskers 1 s.d. and mean line. Data was checked for normality using normality test within OriginPro. All experiments were replicated in multiple rat or human hearts (biologically independent samples/independent experiments) for each condition, indicated by the N number in each figure and figure legend. The exact n values used to calculate statistics and the statistical tests for significance are stated in individual graphs or figure legends. Two-sided t-tests were used to determined statistical significance between experimental conditions vs. control. Where comparisons between sets were both repetitive and restricted, the Bonferroni multiple comparisons correction was used to adjust the significance threshold of two-sided t-tests accordingly (FIGS. 1D to 1G, 1L, and 1M). One-way analysis of variance (ANOVA) with post-hoc Bonferroni test was used to correct multiple comparisons sharing a single control condition (FIGS. 1H to 1J).

### Results

### A Note on Human Samples

Twenty-two human hearts were used in this study for functional tests. All patient studies were conducted on explanted human hearts from patients with non-ischemic heart failure (hypertrophic or dilated cardiomyopathy, HCM or DCM, respectively). When sufficient samples were available, hearts were further sub-classified as either heart failure with reduced ejection fraction (HFrEF) for patients with left-ventricular ejection fraction (LVEF) below 30%, or HFpEF for those with LVEF above 50% at time of transplant. Non-failing (NF) donor hearts were used as controls. For relevant clinical characteristics please see Table 1, and for descriptions of experiments performed on each heart.

TABLE 1
Summary descriptive statistics of human hearts used in this study.

| | No. of | | | |
|---|---|---|---|---|
| Etiology | hearts | Age (year) | LVMI (g/m²) | LVEF (%) |
| NF | 2M, 4F | 54.83 ± 6.09 | 101.03 ± 8.96 | 58.33 ± 5.27 |
| HFpEF | 3M, 3F | 52.17 ± 5.06 | 150.26 ± 10.96 | 67.5 ± 2.81 |
| HFrEF | 6M, 4F | 48.1 ± 3.75 | 116.18 ± 6.49 | 18 ± 1.53 |

Each value is presented as mean ± s.e.m.
Abbreviations: F, female; LVMI, left ventricular mass index; M, male.

TTL Overexpression Improves Contractility in Isolated Cardiomyocytes from Failing Human Hearts Independent of Changes to the Calcium Transient

We previously showed that tubulin tyrosine ligase (TTL) overexpression reduces viscoelasticity and improves contractility in NF human cardiomyocytes.⁵ Because the microtubule network increases in density and becomes highly detyrosinated during heart disease, we wanted to extend these studies to cardiomyocytes from failing hearts, and examine any effect of TTL overexpression on calcium handling. We transduced cardiomyocytes from patients with HF with adenovirus overexpressing TTL (SEQ ID NOs: 1, 22, and 27) or a control virus (null), and assessed electrically stimulated [Ca²⁺]ᵢ transients and sarcomere shortening. Typically, we conduct myocyte contractility assays in the absence of Ca²⁺ indicator dye, as they can slow contractile kinetics due to Ca²⁺ buffering and obscure kinetic phenotypes. For this reason, the [Ca²⁺]ᵢ transient data (in FIGS. 1B to 1G) was collected on a separate subset of cardiomyocytes from those evaluated for contractility. TTL overexpression robustly improved myocyte contraction and relaxation kinetics, with no detectable change to the kinetics or amplitude of the calcium transient (FIG. 1B). These results are consistent with the hypothesis that reducing detyrosination improves the contractility of failing cardiomyocytes through mechanisms predominantly independent of a change in calcium cycling.

Generation of Constructs to Isolate TTL Tubulin Sequestration from Tyrosination

Although TTL overexpression reduces the proportion of detyrosinated microtubules, the precise mechanism by which TTL improves contractility is not clear, as TTL can depolymerize the microtubule network via sequestration of free tubulin,⁸ which is sufficient to improve contractility.⁴ To isolate effects of TTL attributable to tubulin sequestration vs. tyrosination, we generated adenovirus encoding an established TTL catalytic-dead construct (TTL-E331Q) (SEQ ID NO: 28) that binds tubulin with wildtype kinetics, but cannot enzymatically tyrosinate tubulin.⁸ We validated this tool in healthy rat cardiomyocytes, where adenoviral overexpression of TTL-E331Q to the same level of TTL did not significantly reduce detyrosinated tubulin (western blot data is not shown; quantification based on western blot signal is shown in FIGS. 1H and 1I).

### VASH1/SVBP is a Predominant Cardiac Tubulin Carboxypeptidase

To interrogate detyrosination, independent of any potential microtubule depolymerization via TTL, we sought to directly manipulate a cardiac detyrosinase. We conducted experiments to verify if the recently identified VASH-SVBP complex is a primary detyrosinase in myocardium, to determine if any isoform of VASH is dominant, and whether VASH/SVBP expression changes in disease. Transcriptional profiling of healthy and diseased human myocardium identified that VASH1 has approximately 15-fold higher expression level than VASH2 in NF tissue (FIG. 1J). In isolated rat cardiomyocytes, we found Vash1 to be expressed ˜5 fold more than Vash2 (FIG. 1K). In diseased myocardium, VASH1 gene expression was higher in dilated cardiomyopathy (DCM) and did not significantly change in ischemic cardiomyopathy or HCM, while VASH2 gene expression was modestly decreased in HF (FIG. 1J). When comparing the expression of VASH1 to VASH2 across all etiologies of HF, VASH1 was expressed ˜28-50 fold higher than VASH2. SVBP was consistently expressed at a high level and did not show differential expression with disease.

We also sought to evaluate protein abundance of VASH1/2 and SVBP, but were aware of the lack of well-validated antibodies for these targets (note the lack of any such antibodies used in recent publications identifying the role and structure of these enzymes). To address this, we generated new antibodies that were validated in parallel with commercial antibodies in vitro. While the custom VASH1 antibody was indeed more sensitive and specific than commercially available options, we still were unable to detect a reliable band in cardiac cell or tissue lysate. Thus, further work is needed to detect these enzymes, which are likely of relatively low abundance in the cardiac proteome.

We next depleted VASH1, VASH2, and SVBP in healthy rat cardiomyocytes to determine if they function as tubulin carboxypeptidases. For each of these genes, we generated three adenoviruses encoding short hairpin RNAs (shRNAs) specific for three target sites conserved between rats and humans. Delivery of each of these shRNAs by adenovirus to isolated rat cardiomyocytes resulted in a reduction of detyrosinated tubulin, without changing total tubulin levels (western blot not shown; quantification based on western blot signal is shown in FIG. 1M). We selected the shRNAs that resulted in the most robust and consistent decrease in detyrosinated tubulin levels and confirmed that they depleted Vash1, Vash2 and Svbp transcripts via RT-qPCR (FIG. 1L). With similar decreases in transcript levels (FIG. 1L), VASH1 and SVBP KD induced an ˜55% reduction in detyrosinated tubulin while VASH2 KD caused a 25% reduction (FIG. 1M). As SVBP depletion inhibits the functionality of both VASH1 and VASH2,¹⁶⁻¹⁸ the observation that VASH1 and SVBP depletion produced an equivalent reduction in detyrosinated tubulin also suggests that VASH2 has a lesser detyrosinating role in cardiomyocytes. This is consistent with a recent patent¹⁹ demonstrating that VASH1 KO robustly reduces detyrosinated tubulin in the heart, while VASH2 KO has a more modest effect. The fact that TTL overexpression reduces detyrosinated-tubulin by ˜80% while VASH1/SVBP depletion lowers levels by ˜55% is consistent with the abundant expression of TUBA4A in cardiomyocytes,^{5,11} an α-tubulin isotype translated in its detyrosinated form, and thus insensitive to manipulation of a detyrosinase, but readily tyrosinated by TTL.

### Discussion

From the above data we come to the following conclusion: the VASH1-SVBP complex acts as a primary tubulin carboxypeptidase in cardiomyocytes. This work identifies new therapeutic targets for the modulation of cardiomyocyte relaxation and diastolic function. Prior to the discovery of VASH-SVBP as a detyrosinating enzyme complex, the putative carboxypeptidase was commonly inhibited pharmacologically with parthenolide. While effective at high concentrations, parthenolide enacts multiple off target effects including alterations in calcium handling⁵ and cellular signaling cascades.^{10,20} Detyrosination can be genetically reduced by overexpression of the tyrosinating enzyme TTL, and like parthenolide, TTL overexpression reduces stiffness and improves contractility in human cardiomyocytes. Yet the mechanism of TTL action is confounded by the ability of TTL to depolymerize the microtubule network through its 1:1 interaction with free tubulin.^{8,9} While mild depolymerization of the microtubule network might be acutely beneficial in improving cardiomyocyte contractility, chronic or gross depolymerization could lead to trafficking and signaling defects.^{13,21} A TTL-based therapeutic approach is further complicated by a reliance on gene therapy to deliver TTL or the unlikely identification of a TTL agonist. In contrast, small molecule inhibitors of VASH1 or the VASH-SVBP interaction can be more readily designed based on recent structural studies of the complex¹⁶⁻¹⁸ or identified using high-throughput screens, potentially facilitating translational studies. The role of VASH1 as a negative regulator of angiogenesis must be considered, but the vascular architecture of Vash1 KO mice appears largely unchanged.²²

### Example 2—Knockdown of VASH1 Reduces Cardiomyocyte Stiffness and Improves Contractility Independent of the Microtubule Depolymerization

Detyrosination can be genetically decreased by overexpression of tubulin tyrosine ligase (TTL), an enzyme highly specific for tubulin and responsible for ligating the terminal tyrosine residue back to detyrosinated tubulin.⁸ Promisingly, overexpression of TTL in cardiomyocytes from patients with HF reduces cardiomyocyte stiffness, improves contractility,^{3,5} and reduces cell stiffness during diastolic stretch.⁴ However, the precise mechanism by which TTL modulates myocyte mechanics is unclear, as beyond its tyrosinating activity, TTL can also depolymerize microtubules by binding free tubulin dimers, preventing their incorporation into polymerized microtubules.^{8,9} Because it is mechanistically unclear if TTL exerts its effect via changes in microtubule detyrosination, depolymerization, or both, methods to more specifically alter detyrosination are necessary to fully understand the mechanism of TTL action and to better define therapeutic targets for HF. The recent identification of a detyrosinating enzyme complex of vasohibin-1 or -2 (VASH1/2) and their obligate partner small vasohibin binding protein (SVBP) potentially enables the development of more specific approaches to reduce detyrosination.^{11,12} In this study, we confirm that genetic depletion of VASH1 is sufficient to lower stiffness and improve contractility in cardiomyocytes from patients with HF. Further, we identify tyrosination-dependent and independent effects of TTL overexpression and find that tyrosination preferentially improves the relaxation kinetics of cardiomyocytes, and exerts its most profound effects on cardiomyocytes from patients with HF and diastolic dysfunction.

### Nanoindentation

Mechanical properties at the microscopic scale were measured using nanoindentation⁴ (Piuma Chiaro, Optics11, The Netherlands). Freshly isolated human myocytes were attached to glass bottom dishes coated with MyoTak¹³ in normal Tyrode's solution containing 140 mmol/L NaCl, 0.5 mmol/L MgCl₂, 0.33 mmol/L NaH₂PO₄, 5 mmol/L HEPES, 5.5 mmol/L glucose, 1 mmol/L CaCl₂), 5 mmol/L KCl, pH to 7.4 at room temperature. A spherical nano-indentation probe with a radius of 3.05 μm and a stiffness of 0.026 N m⁻¹ was used. Myocytes were indented to a depth of 1.5-3.5 μm with velocities of 0.1, 0.25, 0.5, 1.0, 2.0, 5.0, 10.0, 20.0, 50.0, 100.0, and 150.0 μm s⁻¹. The tip was held in this indentation depth for 1 s, and retracted over 2 s. The Young's moduli were calculated automatically by the software, by fitting the force versus indentation curve to the Hertz equation.

### Statistics

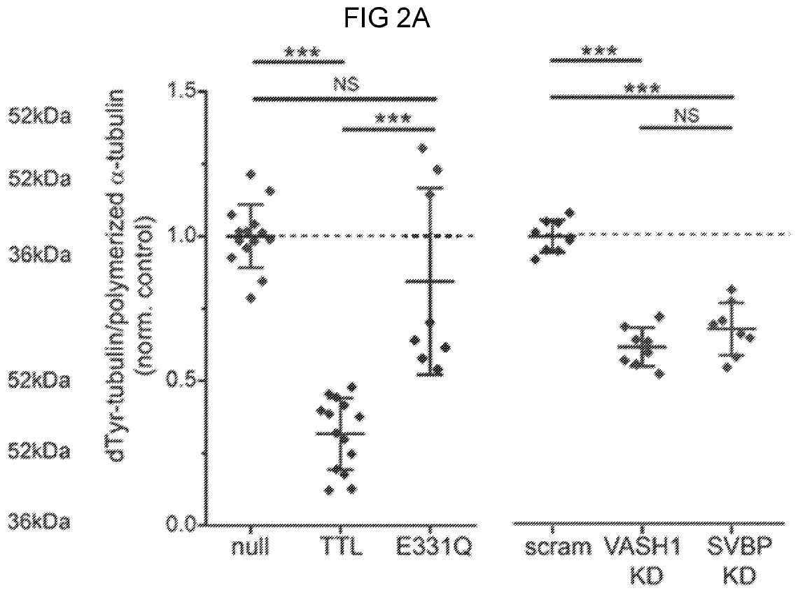

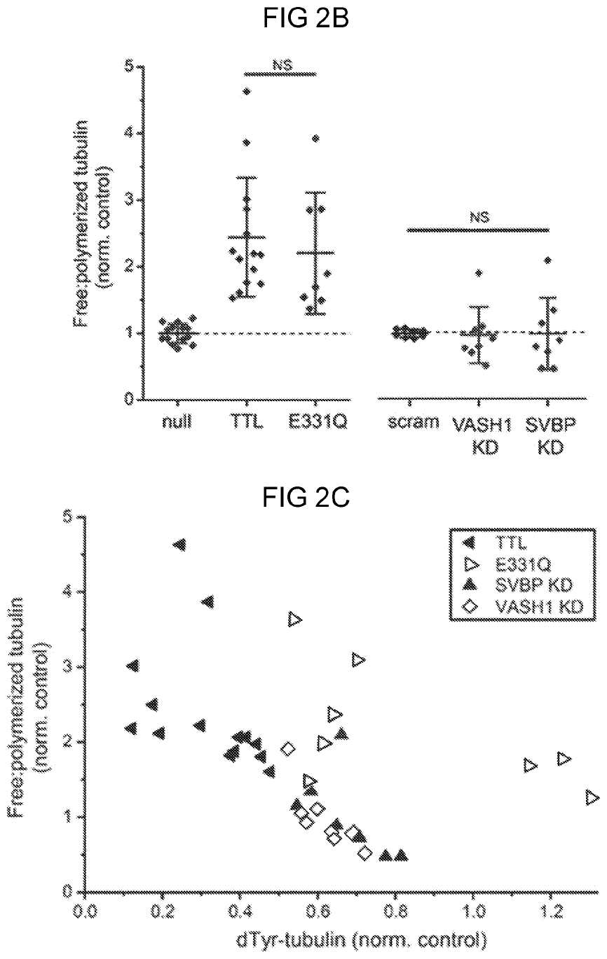

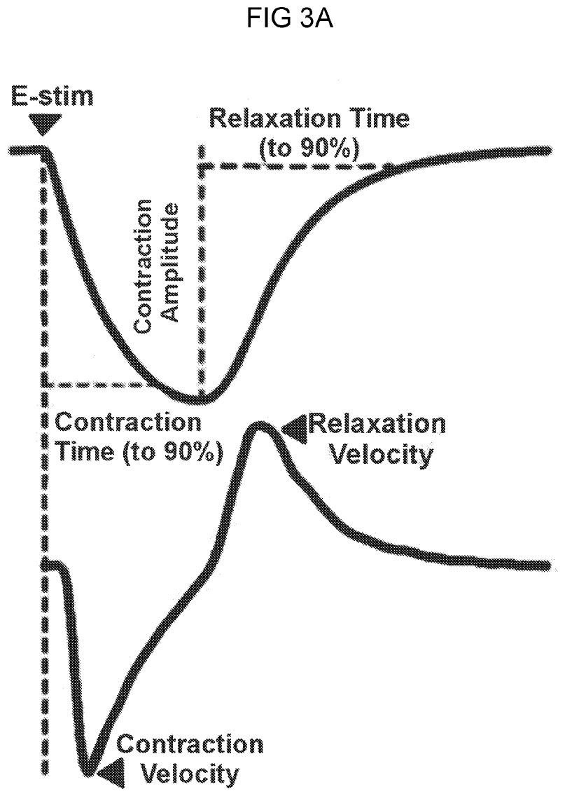

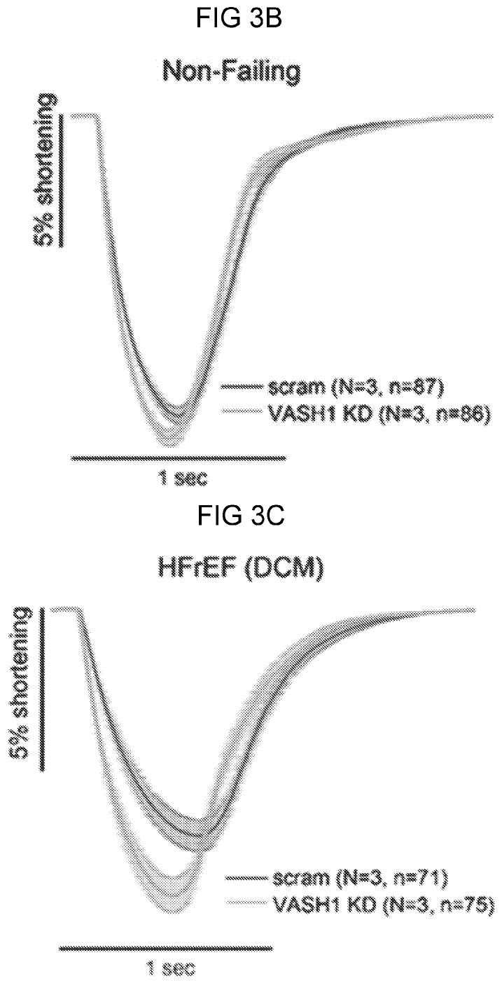

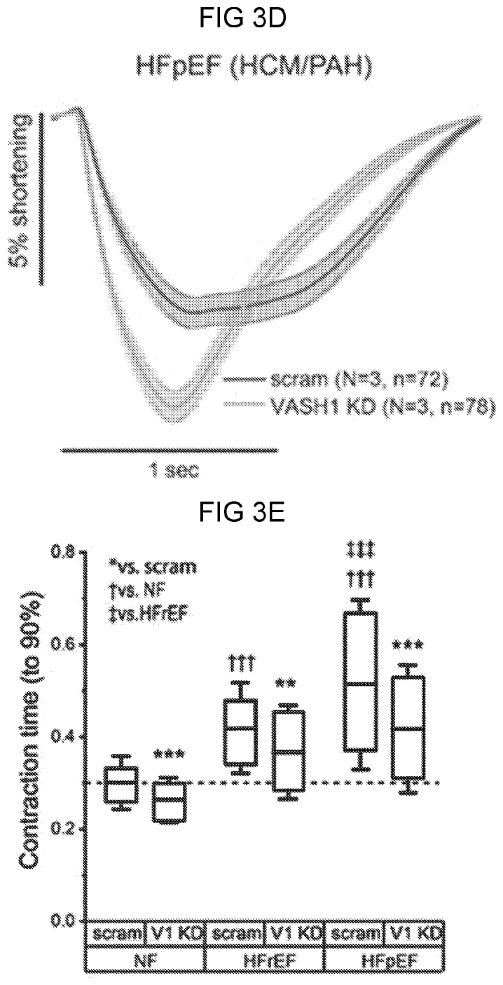

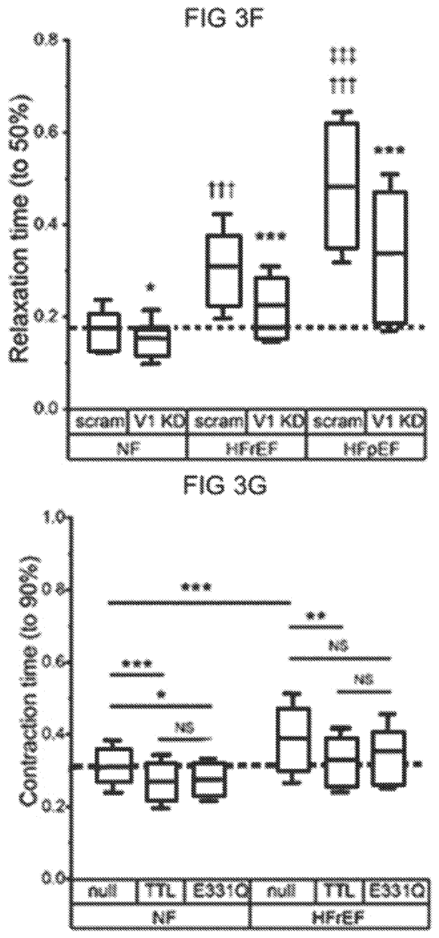

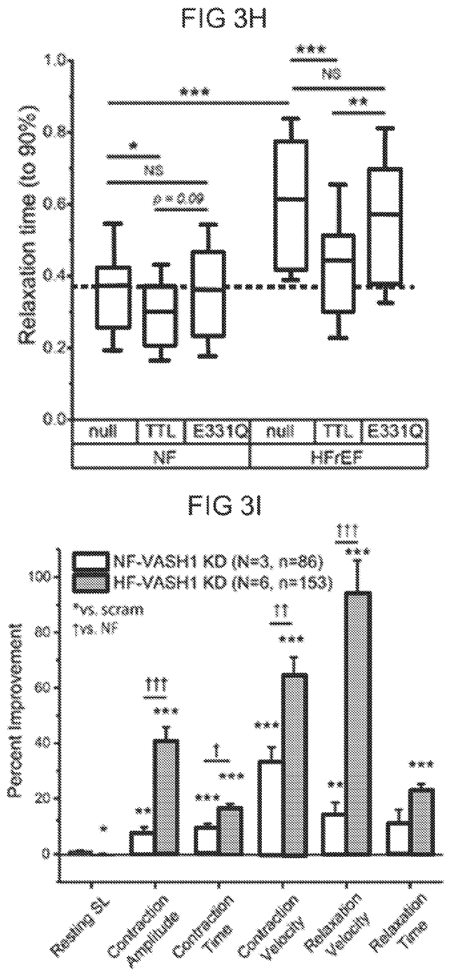

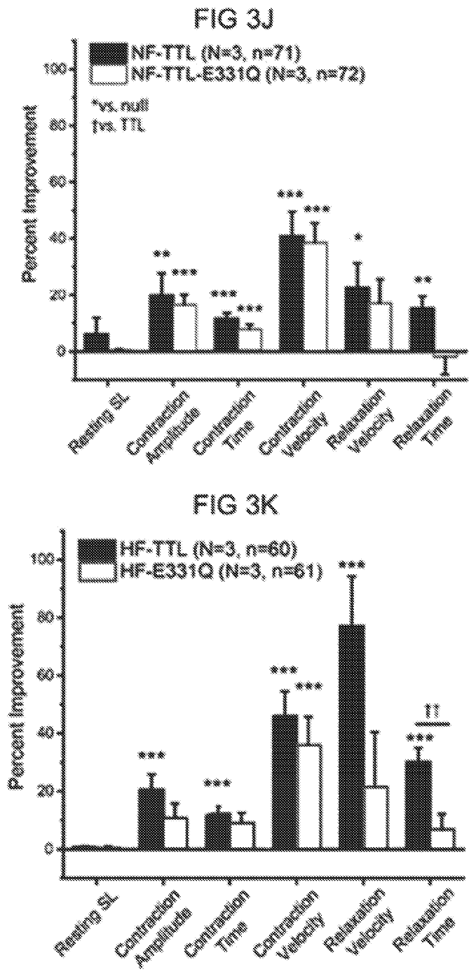

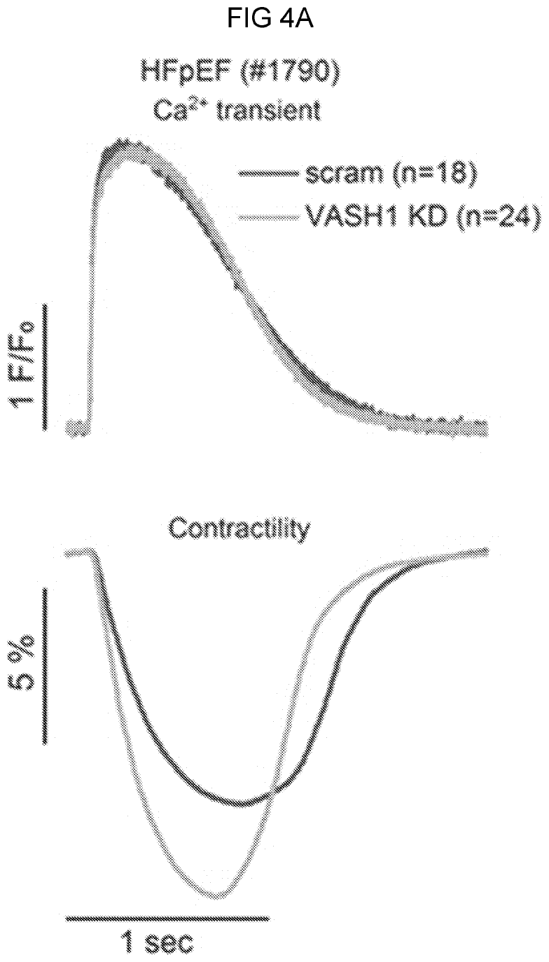

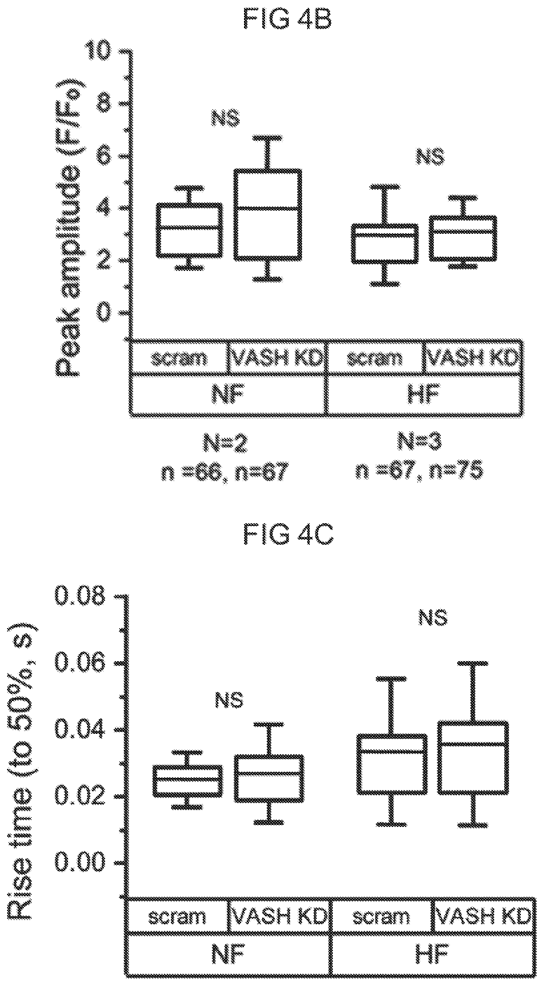

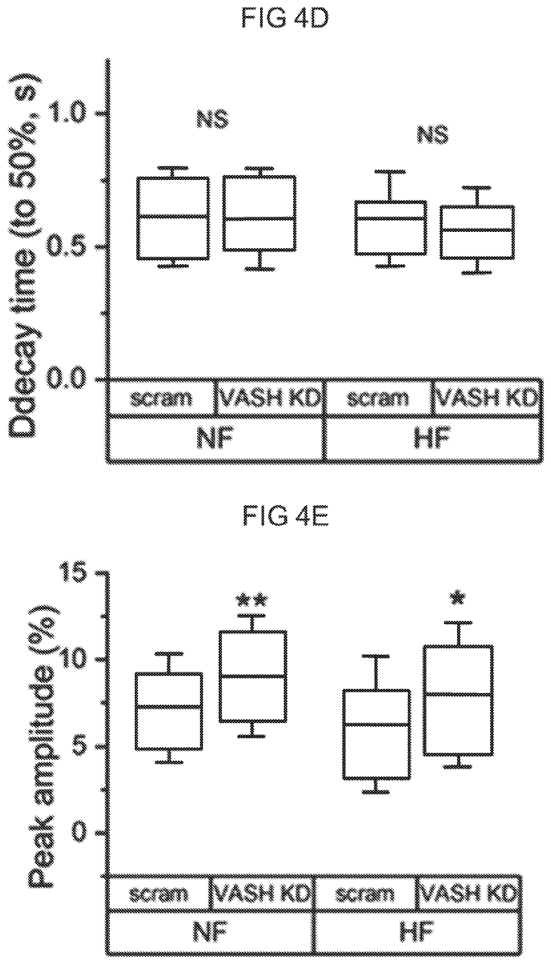

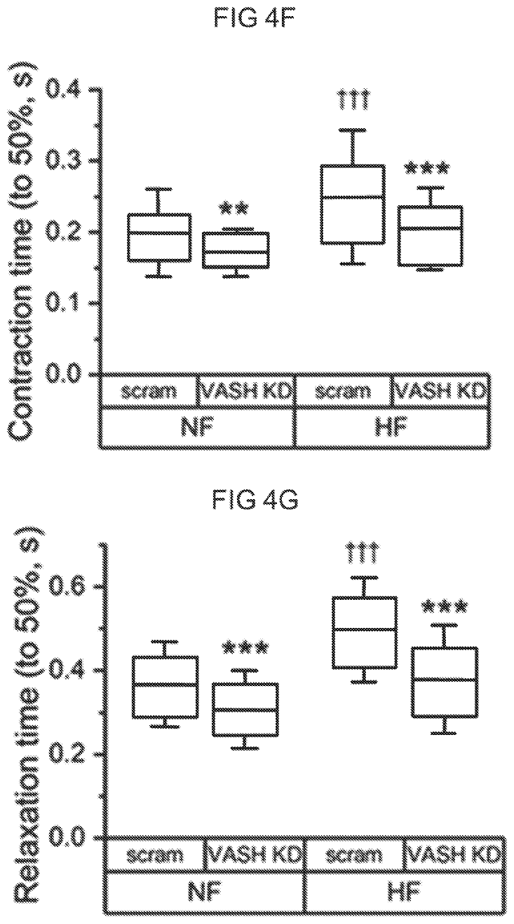

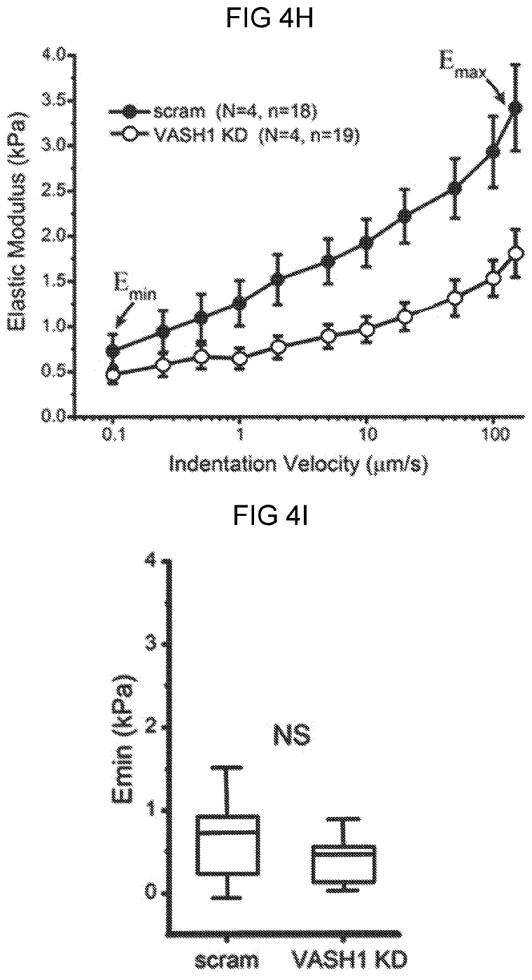

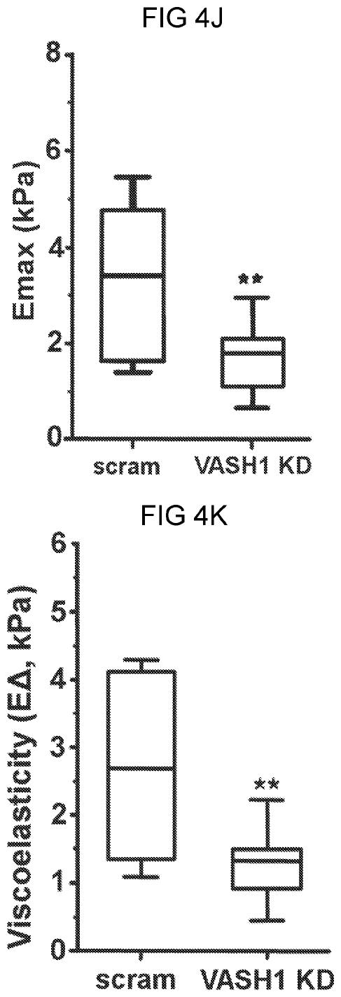

Statistical analysis and graphing were performed using OrginPro software (OriginLabs). Values are presented as mean±s.e.m. in line graphs and bar graphs FIGS. 3I to 3K; FIG. 4I). For box plots, box represents 25^{th} to 75^{th} percentiles, the whiskers 1 s.d. and mean line. Data was checked for normality using normality test within OriginPro. All experiments were replicated in multiple rat or human hearts (biologically independent samples/independent experiments) for each condition, indicated by the N number in each figure and figure legend. The exact n values used to calculate statistics and the statistical tests for significance are stated in individual graphs or figure legends. Two-sided t-tests were used to determined statistical significance between experimental conditions vs. control. Where comparisons between sets were both repetitive and restricted, the Bonferroni multiple comparisons correction was used to adjust the significance threshold of two-sided t-tests accordingly (FIGS. 3E to 3K; FIGS. 4B to 4D). One-way analysis of variance (ANOVA) with post-hoc Bonferroni test was used to correct multiple comparisons sharing a single control condition (FIGS. 2B and 2C; FIGS. 3E to 3H).

### Results

### Tubulin Sequestration by TTL, and not VASH1/SVBP Knockdown, Decreases Microtubule Stability in Healthy Cardiomyocytes

With these new tools generated, we sought to determine the effect that TTL/TTL-E331Q overexpression or VASH1/SVBP knockdown has on the stability of the microtubule network. Using a fractionation assay that allows for separation of soluble, free tubulin from polymerized microtubules, we found that overexpression of TTL (˜10 fold) in healthy rat cardiomyocytes led to a robust (˜80%) reduction in detyrosinated tubulin that did not occur with TTL-E331Q (FIG. 2A). Both TTL and TTL-E331Q overexpression increased the amount of free tubulin relative to polymerized microtubules (FIGS. 2A and 2B), indicating modest depolymerization of the microtubule network. In contrast, knockdown of either VASH1 or SVBP reduced the level of detyrosinated tubulin in polymerized microtubules (˜40%), yet the free:polymerized tubulin ratio remained unchanged (FIGS. 2A and 2B). These results suggest that tubulin binding via TTL is sufficient to depolymerize the microtubule network in isolated rat cardiomyocytes, and that modest reductions in detyrosination alone are not sufficient to depolymerize microtubules.

We noted that TTL overexpression or knockdown resulted in variable changes in the ratio of free:polymerized tubulin that seemed to mirror changes in detyrosination. Indeed, plotting the change in detyrosinated tubulin from each experiment against the change in free:polymerized tubulin revealed an inverse correlation with levels of detyrosination (FIG. 2C), suggesting that decreasing detyrosination may decrease microtubule network stability in cardiomyocytes, but requires a threshold reduction in detyrosination. This would imply that any destabilizing effect of VASH1/SVBP depletion would be contingent on a sufficient initial proportion of detyrosinated microtubules in the network, such as that occurs in HF.

### Effects of VASH1 Knockdown and TTL-E331Q Overexpression on Myocyte Contractility

We next sought to determine how TTL/TTL-E331Q overexpression or VASH1 knockdown would affect the contractility of isolated cardiomyocytes from failing and NF hearts. In cardiomyocytes from NF donor hearts, VASH1 knockdown produced modest but consistent speeding of contraction and relaxation kinetics, and subtle effects on contractile amplitudes (FIGS. 3A to 3I). We also interrogated the effect of VASH1 depletion in cardiomyocytes from 6 patients with HF, 3 with HFrEF (systolic dysfunction), and 3 with HFpEF (diastolic dysfunction). As shown in the average traces of cardiomyocytes from these hearts transduced with control adenovirus (FIGS. 3B to 3D), failing heart cells exhibited lower contractile amplitudes and slower kinetics than NF controls. There was markedly slower relaxation in failing cardiomyocytes that was particularly evident in HFpEF cardiomyocytes (FIGS. 3E to 3H), consistent with the impaired relaxation implicated in this disease etiology. On average, relaxation velocity was 2.1-fold and 3.6-fold lower in HFrEF and HFpEF than in NF controls, respectively. VASH1 depletion markedly improved contractile amplitudes and contraction and relaxation kinetics in failing cardiomyocytes, eliciting a 1.6-fold and 2-fold improvement in relaxation velocity in HFrEF and HFpEF cardiomyocytes, respectively (FIGS. 3A to 3I).

The effect of SVBP depletion was examined in cardiomyocytes from a separate subset of 3 failing hearts (2 HFrEF, 1HFpEF), where it elicited similar improvements in contraction and relaxation kinetics as VASH1 knockdown.

In NF cardiomyocytes, both TTL (SEQ ID NO. 27) and TTL-E331Q (SEQ ID NO. 28) overexpression improved most metrics of systolic function compared to cells transduced with control adenovirus, although improvements in relaxation time appeared to only be sensitive to TTL, and not to TTL-E331Q (FIGS. 3G, 3H and 3J). This became more apparent in cardiomyocytes from failing hearts (HFrEF), where both constructs improved systolic parameters, yet TTL had a significantly greater effect than its catalytically dead counterpart on relaxation time, and robustly improved relaxation velocity (FIGS. 3G, 3H and 3K).

Together, this data indicates that 1) relaxation kinetics are particularly slowed in failing cardiomyocytes; 2) selectively targeting microtubule detyrosination via either the tyrosinase or detyrosinase is sufficient to robustly improve relaxation kinetics; 3) the effect of reducing microtubule detyrosination is most remarkable in failing cardiomyocytes; and 4) the microtubule depolymerizing activity of TTL is sufficient to augment systolic, but not necessarily diastolic parameters.

### VASH-1 Knockdown Reduces Myocyte Stiffness and has Negligible Effects on [Ca²⁺]; Transients

Typically, we conduct myocyte contractility assays in the absence of Ca²⁺ indicator dye, as they can slow contractile kinetics due to Ca²⁺ buffering and obscure kinetic phenotypes. Recognizing that inhibition of tubulin detyrosination with parthenolide has off target consequences on calcium handling in human cardiomyocytes,⁵ we sought to more directly examine the relationship between calcium handling and contractility after VASH1 knockdown in myocytes. Thus, cardiomyocytes from a separate subset of 5 hearts were loaded with a low concentration of the Ca²⁺ indicator dye fluo-3 and simultaneously assessed for [Ca²⁺]ᵢ transients and contractility. As shown in the average trace of myocytes from a HFpEF heart (FIG. 4A), VASH1 knockdown robustly improved fractional shortening, contraction and relaxation speed independent of any observable change in Ca²⁺ handling in those same cells. This Ca²⁺-independent improvement in contractility was observed in both NF and failing cardiomyocytes (FIGS. 4B to 4G).

In the absence of any demonstrable effect on Ca²⁺ cycling, the observed improvement of systolic and diastolic kinetics with VASH1 knockdown are consistent with a reduction of viscoelastic resistance to sarcomere motion contributed by detyrosinated microtubules.⁴ To test this hypothesis, we directly probed viscoelasticity via transverse nanoindentation of failing human cardiomyocytes with or without VASH1 knockdown. In response to very slow deformation, the Young's modulus of VASH1-depleted cells was unchanged, suggesting a minimal effect on transverse elasticity (Eₘᵢₙ, FIG. 4I). Yet at rates of deformation relevant to the cardiac cycle, VASH1 knockdown reduced transverse stiffness, with a prominent reduction in peak stiffness at high speeds of deformation (Eₘₐₓ) (FIG. 4J) and viscoelasticity (EΔ, rate-dependent stiffness) (FIG. 4K) in cardiomyocytes from failing human hearts. Together, these data are consistent with improved kinetics of failing ventricular cardiomyocytes conferred at least in part by reductions in viscoelasticity, due to depletion of VASH1 and reduction in detyrosinated microtubules.

### Discussion

From the above data we come to the following conclusions: 1) knockdown of VASH1 reduces cardiomyocyte stiffness and improves contractility, particularly in cardiomyocytes from patients with HF; and 2) targeting microtubule detyrosination specifically speeds cardiomyocyte relaxation, which does not require microtubule depolymerization or changes to the Ca²⁺ transient. This work identifies new therapeutic targets for the modulation of cardiomyocyte relaxation and diastolic function. This study represents the first examination of direct suppression of a tubulin carboxypeptidase in cardiomyocytes.

VASH1 depletion had minor effects on the contractile profile of NF cardiomyocytes, with more robust effects on cardiomyocytes from patients with HF, particularly HFpEF where relaxation kinetics were dramatically slowed. Diastolic dysfunction is a hallmark of both HCM and HFpEF, where both intrinsic myocyte adaptations and extrinsic changes can impair myocardial relaxation.^{1,2} Reducing cardiomyocyte stiffness and improving myocyte relaxation could reduce isovolumic relaxation times, increase ventricular compliance, and reduce diastolic pressures, all of which may benefit patients with diastolic dysfunction. Pharmacological approaches to promote myocyte relaxation and treat HCM are in development, including calcium desensitizers^{23,24} and most notably with myosin inhibitors that have advanced to clinical trials.²⁵ Interestingly, myosin inhibitors speed relaxation proportional to a reduction in contractility, as do interventions that reduce myofilament calcium sensitivity. It is unclear if the therapeutic benefit of such approaches arises from the faster relaxation, reduced systolic force production, or a net decrease in time under tension that may limit hypertrophic remodeling. Intriguingly, our results indicate an alternative, Ca²⁺-independent mechanism to speed relaxation. Reducing viscoelasticity (energy loss) may speed cardiomyocyte relaxation without compromising systolic force production or increasing energetic demand, as would be the case for an increase in calcium cycling. How such a strategy would fit in the toolkit and compare to current approaches for treating different etiologies of diastolic or systolic dysfunction is of interest and will require extensive further study.

A limitation of the current work is the difficulty in quantifying protein levels of VASH/SVBP, which may be aided by further antibody refinement or targeted mass-spectrometry approaches. Additionally, protein expression does not speak to enzymatic activity directly, and little is known about post-translational regulation of these enzymes and the effect it has on detyrosinase activity.

These results provide the first demonstration of the effects of VASH1/SVBP inhibition on the intrinsic relaxation and viscoelasticity of isolated cardiomyocytes. This is a simplified system, and the diastolic pressure-volume relationship in vivo is influenced by numerous additional factors, including filling pressures that load and stretch cardiomyocytes. Of note, similar reductions in viscoelasticity with parthenolide or TTL overexpression equate to increased compliance of cardiomyocytes upon loaded cell stretch,⁴ so we surmise the same will hold true for inhibition of VASH1. The current study prompts future investigations of the role of VASH1 in animal models of diastolic dysfunction, yet care must be taken in experimental design.

It is difficult to predict how chronic manipulations of TTL may affect cardiac function (or how effects will be interpreted), given the numerous potential consequences of altering microtubule detyrosination,²⁶ for example on autophagy,²⁷ oxidative stress,^{13,28} and intracellular trafficking.²⁹⁻³¹ Further, it is unclear whether common murine models of heart failure recapitulate the cytoskeletal remodeling that occurs in patients with HCM and heart failure.⁵⁻⁷ Studies utilizing small molecule inhibition or rapid, inducible depletion of VASH1 in models of diastolic dysfunction that recapitulate the patient cytoskeletal landscape will be most informative.

### REFERENCES

1. Rakowski H, Carasso S. Quantifying Diastolic Function in Hypertrophic Cardiomyopathy. Circulation. 2007; 116:2662-2665.
2. Borlaug B A, Paulus W J. Heart failure with preserved ejection fraction: pathophysiology, diagnosis, and treatment. Eur Heart J. 2011; 32:670-679.
3. Robison P, Caporizzo M A, Ahmadzadeh H, Bogush A I, Chen C, Margulies K B, Shenoy V B, Prosser B L. Detyrosinated microtubules buckle and bear load in contracting cardiomyocytes. Science. 2016; 352:aafO659.
4. Caporizzo M, Chen C, Salomon A, Margulies K B, Prosser B L. Microtubules provide a viscoelastic resistance to myocyte motion. Biophys J. 2018; 115:1796 1807.
5. Chen C, Caporizzo M A, Bedi K, Vite A, Bogush A I, Robison P, Heffler J G, Salomon A K, Kelly N A, Babu A, Morley M P, Margulies K B, Prosser B L. Suppression of detyrosinated microtubules improves cardiomyocyte function in human heart failure. Nat Med. 2018; 24:1225-1233.
6. Zile M R, Green G R, Schuyler G T, Aurigemma G P, Miller D C, Cooper G. Cardiocyte cytoskeleton in patients with left ventricular pressure overload hypertrophy. J Am Coll Cardiol. 2001; 37:1080-1084.
7. Dorsch L M, Schuldt M, dos Remedios C G, Schinkel A, de Jong P L, Michels M, Kuster D, Brundel B, van der Velden J. Protein Quality Control Activation and Microtubule Remodeling in Hypertrophic Cardiomyopathy. Cells. 2019; 8:741.
8. Szyk A, aconescu A, Piszczek G, Roll-Mecak A. Tubulin tyrosine ligase structure reveals adaptation of an ancient fold to bind and modify tubulin. Nat Struct Mol Biol. 2011; 18:1250.
9. Nieuwenhuis J, Brummelkamp T R. The Tubulin Detyrosination Cycle: Function and Enzymes. Trends Cell Biol. 2018; 29:80-92.
10. Wen J, You K-R, Lee S-Y, Song C-H, Kim D-G. Oxidative Stress-mediated Apoptosis THE ANTICANCER EFFECT OF THE SESQUITERPENE LACTONE PARTHENOLIDE. J Biol Chem. 2002; 277:38954-38964.
11. Aillaud C, Bose C, Peris L, Bosson A, Heemeryck P, Dijk J, Friec J, Boulan B, Vossier F, Sanman L E, Syed S, Amara N, Couté Y, Lafanechbre L, Denarier E, Delphin C, Pelletier L, Humbert S, Bogyo M, Andrieux A, Rogowski K, Moutin M-J. Vasohibins/SVBP are tubulin carboxypeptidases (TCPs) that regulate neuron differentiation. Science. 2017; 358:1448-1453.
12. Nieuwenhuis J, Adamopoulos A, Bleijerveld O B, Mazouzi A, Stickel E, Celie P, Altelaar M, Knipscheer P, Perrakis A, Blomen V A, Brummelkamp T R. Vasohibins encode tubulin detyrosinating activity. Science. 2017; 358:1453-1456.
13. Prosser B L, Ward C W, Lederer W. X-ROS Signaling: Rapid Mechano-Chemo Transduction in Heart. Science. 2011; 333:1440-1445.
14. Heffler J, Shah P P, Robison P, Phyo S, Veliz K, Uchida K, Bogush A, Rhoades J, Jain R, Prosser B L. A Balance Between Intermediate Filaments and Microtubules Maintains Nuclear Architecture in the Cardiomyocyte. Circ Res. 2019;
15. Singh S R, Zech A, Geertz B, Reischmann-Düsener S, Osinska H, Prondzynski M, Krämer E, Meng Q, Redwood C, van der Velden J, Robbins J, Schlossarek S, Carrier L. Activation of Autophagy Ameliorates Cardiomyopathy in Mybpc3-Targeted Knockin Mice. Circulation Hear Fail. 2018; 10:e004140.
16. Wang N, Bose C, Choi S, Boulan B, Peris L, Olieric N, Bao H, Krichen F, Chen L, Andrieux A, Olieric V, Moutin M-J, Steinmetz M O, Huang H. Structural basis of tubulin detyrosination by the vasohibin-SVBP enzyme complex. Nat Struct Mol Biol. 2019; 1-12.
17. Li F, Hu Y, Qi S, Luo X, Yu H. Structural basis of tubulin detyrosination by vasohibins. Nat Struct Mol Biol. 2019; 26:583-591.
18. Liao S, Rajendraprasad G, Wang N, Eibes S, Gao J, Yu H, Wu G, Tu X, Huang H, Barisic M, Xu C. Molecular basis of vasohibins-mediated detyrosination and its impact on spindle function and mitosis. Cell Res. 2019; 1-15.
19. Andrieux A, Moutin M-J, Bose C, Aillaud C, Peris L, Delagrange P, inventors; INSERM, Universite Grenoble Alpes, Commissariat A L'energie Atomique Et Aux Energies Alternatives, Les Laboratoires Servier, assignees. Methods and pharmaceutical compositions for treating tubulin carboxypeptidases associated diseases. World International Patent Number: PCT/EP2018/079448. 2018 Oct. 26.
20. Ghantous A, Sinjab A, Herceg Z, Darwiche N. Parthenolide: from plant shoots to cancer roots. Drug Discov Today. 2013; 18:894-905.
21. Shaw R M, Fay A J, Puthenveedu M A, von Zastrow M, Jan Y-N, Jan L Y. Microtubule Plus-End-Tracking Proteins Target Gap Junctions Directly from the Cell Interior to Adherens Junctions. Cell. 2007; 128:547-560.
22. Kimura H, Miyashita H, Suzuki Y, Kobayashi M, Watanabe K, Sonoda H, Ohta H, Fujiwara T, Shimosegawa T, Sato Y. Distinctive localization and opposed roles of vasohibin-1 and vasohibin-2 in the regulation of angiogenesis. Blood. 2009; 113:4810-4818.
23. Tadano N, Du C, Yumoto F, Morimoto S, Ohta M, Xie M, Nagata K, Zhan D, Lu Q, Miwa Y, Takahashi-Yanaga F, Tanokura M, Ohtsuki I, Sasaguri T. Biological actions of green tea catechins on cardiac troponin C. Brit J Pharmacol. 2010; 161:1034-1043.
24. Adhikari B B, Wang K. Interplay of Troponin- and Myosin-Based Pathways of Calcium Activation in Skeletal and Cardiac Muscle: The Use of W7 as an Inhibitor of Thin Filament Activation. Biophys J. 2004; 86:359-370.
25. Green E M, Wakimoto H, Anderson R L, Evanchik M J, Gorham J M, Harrison B C, Henze M, Kawas R, Oslob J D, Rodriguez H M, Song Y, Wan W, Leinwand L A, Spudich J A, well R S, Seidman J, Seidman C E. A small-molecule inhibitor of sarcomere contractility suppresses hypertrophic cardiomyopathy in mice. Science. 2016; 351:617-621.
26. Caporizzo M A, Chen C, Prosser B L. Cardiac microtubules in health and heart disease. Exp Biol Med. 2019; 153537021986896.
27. Mohan N, Sorokina E M, Verdeny I, Alvarez A, Lakadamyali M. Detyrosinated microtubules spatially constrain lysosomes facilitating lysosome-autophagosome fusion. J Cell Biol. 2018; 218:jcb.201807124.
28. Kerr J P, Robison P, Shi G, Bogush A I, Kempema A M, Hexum J K, Becerra N, Harki D A, Martin S S, Raiteri R, Prosser B L, Ward C W. Detyrosinated microtubules modulate mechanotransduction in heart and skeletal muscle. Nat Commun. 2015; 6:8526.
29. Sirajuddin M, Rice L M, Vale R D. Regulation of microtubule motors by tubulin isotypes and post-translational modifications. Nat Cell Biol. 2014; 16:335-344.
30. Barisic M, e Sousa R, Tripathy S K, Magiera M M, Zaytsev A V, Pereira A L, Janke C, Grishchuk E L, Maiato H. Microtubule detyrosination guides chromosomes during mitosis. Science. 2015; 348:799-803.
31. Hammond J W, Huang C-F, Kaech S, Jacobson C, Banker G, Verhey K J. Posttranslational Modifications of Tubulin and the Polarized Transport of Kinesin-1 in Neurons. Mol Biol Cell. 2010; 21:572-583.

All patents, patent applications, and publications, and references to GenBank or another publicly available sequences database cited throughout the disclosure, are expressly incorporated herein by reference in its entirety. U.S. Provisional Patent Application No. 62/990,147, filed Mar. 16, 2020 is incorporated herein by reference. The Sequence listing filed herewith, labeled 20-9311PCT_ST25.txt, and the sequences and text therein is incorporated by reference. While this invention has been disclosed with reference to specific embodiments, it is apparent that other embodiments and variations of this invention are devised by others skilled in the art without departing from the true spirit and scope of the invention. The appended claims include such embodiments and equivalent variations.

### SEQUENCE LISTING FREE TEXT

The following information is provided for sequences containing free text under numeric identifier <223>.

| SEQ ID NO: | Free Text under <223> |
|---|---|
| 1 | <223> plasmid comprising AAV vector genome for |
| | expression of TTL |
| | <220> |
| | <221> repeat_region |
| | <222> (1)..(130) |
| | <223> 5′UTR |
| | <220> |
| | <221> promoter |
| | <222> (214)..(655) |
| | <223> cTNT cardiac specific promoter |
| | <220> |
| | <221> Intron |
| | <222> (772)..(904) |
| | <223> Intron 1 |
| | <220> |
| | <221> CDS |
| | <222> (997)..(2160) |
| | <223> TTLcds |
| | <220> |
| | <221> RBS |
| | <222> (2194)..(2778) |
| | <223> IRES |
| | <220> |
| | <221> CDS |
| | <222> (2782)..(3459) |
| | <223> dsRED |
| | <220> |
| | <221> misc_feature |
| | <222> (3523)..(4110) |
| | <223> WPRE (Woodchuck Hepatitis Virus |
| | Posttranscriptional Regulatory |
| | Element) |
| | <220> |
| | <221> misc_feature |
| | <222> (4132)..(4258) |
| | <223> Rabbit globin poly A |
| | <220> |
| | <221> repeat_region |
| | <222> (4347)..(4476) |
| | <223> 3′ITR |
| | <220> |
| | <221> misc_feature |
| | <222> (4653)..(5108) |
| | <223> fl ori |
| | <220> |
| | <221> misc_feature |
| | <222> (5239)..(6096) |
| | <223> AP(R) |
| | <220> |
| | <221> rep_origin |
| | <222> (6270)..(6858) |
| | <223> Origin of replication |
| 2 | <223> Synthetic Construct |
| 3 | <223> Synthetic Construct |
| 4 | <223> plasmid comprisisng an entry clone vector for |
| | use in adenoviral |
| | destination vector for production of adenoviruses |
| | encoding shRNA |
| | (i.e. shVASH1-1) |
| | <220> |
| | <221> rep_origin |
| | <222> (87)..(760) |
| | <223> pUC origin |
| | <220> |
| | <221> terminator |
| | <222> (1031)..(1058) |
| | <223> rrnB T2 transcription terminator |
| | <220> |
| | <221> terminator |
| | <222> (1190)..(1233) |
| | <223> rrnB Tit transcription terminator |
| | <220> |
| | <221> primer bind |
| | <222> (1300)..(1315) |
| | <223> M13 Forward priming site |
| | <220> |
| | <221> misc_recomb |
| | <222> (1332)..(1431) |
| | <223> aatL1 |
| | <220> |
| | <221> promoter |
| | <222> (1468)..(1731) |
| | <223> U6 Promotor (RNA Pol III) |
| | <220> |
| | <221> primer bind |
| | <222> (1653)..(1672) |
| | <223> U6 Forward priming site |
| | <220> |
| | <221> misc_feature |
| | <222> (1728)..(1731) |
| | <223> shRNA 5′ overhang |
| | <220> |
| | <221> misc_RNA |
| | <222> (1732)..(1777) |
| | <223> shVASH1 sh1 (shRNA) |
| | <220> |
| | <221> misc_feature |
| | <222> (1732)..(1752) |
| | <223> Stem (targeting shRNA) |
| | <220> |
| | <221> misc_feature |
| | <222> (1753)..(1756) |
| | <223> Loop (targeting shRNA) |
| | <220> |
| | <221> misc_feature |
| | <222> (1757)..(1777) |
| | <223> Stem (targeting shRNA) |
| | <220> |
| | <221> terminator |
| | <222> (1778)..(1783) |
| | <223> Pol III T(Pol III Transcription terminator) |
| | <220> |
| | <221> misc_feature |
| | <222> (1778)..(1781) |
| | <223> 5′ overhang (targeting shRNA) |
| | <220> |
| | <221> misc_recomb |
| | <222> (1788)..(1887) |
| | <223> attl2 |
| | <220> |
| | <221> primer bind |
| | <222> (1928)..(1944) |
| | <223> M13 Reverse Priming Site |
| | <220> |
| | <221> CDS |
| | <222> (2057)..(2866) |
| | <223> Kan (Kanamycin Resistance Gene) |
| 5 | <223> Synthetic Construct |
| 6 | <223> plasmid comprisisng an entry clone vector for |
| | use in adenoviral |
| | destination vector for production of adenoviruses |
| | encoding shRNA |
| | (i.e. shVASH1-2) |
| | <220> |
| | <221> rep_origin |
| | <222> (90)..(763) |
| | <223> pUC ori |
| | <220> |
| | <221> terminator |
| | <222> (1034)..(1061) |
| | <223> rrnB T2 (transcription terminator) |
| | <220> |
| | <221> terminator |
| | <222> (1193)..(1236) |
| | <223> rrnR Tit (trascription terminator) |
| | <220> |
| | <221> protein bind |
| | <222> (1303)..(1318) |
| | <223> M13 Forward priming cite |
| | <220> |
| | <221> misc_recomb |
| | <222> (1335)..(1434) |
| | <223> attL1 |
| | <220> |
| | <221> promoter |
| | <222> (1471)..(1734) |
| | <223> U6 promotor (RNA pol III) |
| | <220> |
| | <221> primer bind |
| | <222> (1656)..(1675) |
| | <223> U6 Forward priming site |
| | <220> |
| | <221> misc_feature |
| | <222> (1731)..(1734) |
| | <223> 5′ overhang |
| | <220> |
| | <221> misc_RNA |
| | <222> (1735)..(1780) |
| | <223> shVASH1 sh2 (shRNA) |
| | <220> |
| | <221> misc_feature |
| | <222> (1735)..(1755) |
| | <223> Stem (targeting shRNA) |
| | <220> |
| | <221> misc_feature |
| | <222> (1756)..(1759) |
| | <223> Loop (targeting shRNA) |
| | <220> |
| | <221> misc_feature |
| | <222> (1760)..(1780) |
| | <223> Stem (targeting shRNA) |
| | <220> |
| | <221> terminator |
| | <222> (1781)..(1786) |
| | <223> Pol III T (Pol III Transcription Terminator) |
| | <220> |
| | <221> misc_feature |
| | <222> (1781)..(1784) |
| | <223> 5′ overhang |
| | <220> |
| | <221> misc_recomb |
| | <222> (1791)..(1890) |
| | <223> attL2 |
| | <220> |
| | <221> primer bind |
| | <222> (1931)..(1947) |
| | <223> M13 Reverse priming site |
| | <220> |
| | <221> CDS |
| | <222> (2060)..(2869) |
| | <223> Kanamycin Resistance Gene |
| 7 | <223> Synthetic Construct |
| 8 | <223> plasmid comprisisng an entry clone vector for |
| | use in adenoviral |
| | destination vector for production of adenoviruses |
| | encoding shRNA |
| | (i.e. shVASH1-3) |
| | <220> |
| | <221> rep_origin |
| | <222> (63)..(736) |
| | <223> pUC origin |
| | <220> |
| | <221> terminator |
| | <222> (1007)..(1034) |
| | <223> rrnB T2 transcription terminator |
| | <220> |
| | <221> terminator |
| | <222> (1166)..(1209) |
| | <223> rrnB Tit transcription temrinator |
| | <220> |
| | <221> primer bind |
| | <222> (1276)..(1291) |
| | <223> M13 Forward primer bind |
| | <220> |
| | <221> misc_recomb |
| | <222> (1308)..(1407) |
| | <223> attL1 |
| | <220> |
| | <221> promoter |
| | <222> (1444)..(1707) |
| | <223> U6 Promotor (RNA Pol III) |
| | <220> |
| | <221> primer bind |
| | <222> (1629)..(1648) |
| | <223> U6 Forward priming site |
| | <220> |
| | <221> misc_feature |
| | <222> (1704)..(1707) |
| | <223> 5′ overhang |
| | <220> |
| | <221> misc_RNA |
| | <222> (1708)..(1753) |
| | <223> shVASH1 sh3 (shRNA) |
| | <220> |
| | <221> misc_feature |
| | <222> (1708)..(1728) |
| | <223> Stem (targeting shRNA) |
| | <220> |
| | <221> misc_feature |
| | <222> (1729)..(1732) |
| | <223> Loop (targeting shRNA) |
| | <220> |
| | <221> misc_feature |
| | <222> (1733)..(1753) |
| | <223> Stem (targeting shRNA) |
| | <220> |
| | <221> terminator |
| | <222> (1754)..(1759) |
| | <223> Pol III T (Pol III Transcription Terminator) |
| | <220> |
| | <221> misc_feature |
| | <222> (1754)..(1757) |
| | <223> 5′ overhang |
| | <220> |
| | <221> misc_recomb |
| | <222> (1764)..(1863) |
| | <223> attL2 |
| | <220> |
| | <221> primer bind |
| | <222> (1904)..(1920) |
| | <223> M13 reverse priming site |
| | <220> |
| | <221> CDS |
| | <222> (2033)..(2842) |
| | <223> Kanamycin Resistance Gene |
| 9 | <223> Synthetic Construct |
| 10 | <223> plasmid comprisisng an entry clone vector for |
| | use in adenoviral |
| | destination vector for production of adenoviruses |
| | encoding shRNA |
| | (i.e. shSVBP-1) |
| | <220> |
| | <221> rep_origin |
| | <222> (74)..(747) |
| | <223> pUC origin |
| | <220> |
| | <221> terminator |
| | <222> (1018)..(1045) |
| | <223> rrnB T2 transcription terminator |
| | <220> |
| | <221> terminator |
| | <222> (1177)..(1220) |
| | <223> rrnB Tit transcription terminator |
| | <220> |
| | <221> primer bind |
| | <222> (1287)..(1302) |
| | <223> M13 Forward priming site |
| | <220> |
| | <221> misc_recomb |
| | <222> (1319)..(1418) |
| | <223> attLl |
| | <220> |
| | <221> promoter |
| | <222> (1455)..(1718) |
| | <223> U6 Promotor (RNA Pol III) |
| | <220> |
| | <221> primer bind |
| | <222> (1640)..(1659) |
| | <223> U6 Priming site |
| | <220> |
| | <221> misc_feature |
| | <222> (1715)..(1718) |
| | <223> 5′ overhang |
| | <220> |
| | <221> misc_RNA |
| | <222> (1719)..(1764) |
| | <223> shSVBP sh1 (shRNA SVBP1) |
| | <220> |
| | <221> misc_feature |
| | <222> (1740)..(1743) |
| | <223> Loop (targeting shRNA) |
| | <220> |
| | <221> terminator |
| | <222> (1765)..(1770) |
| | <223> Pol III T (Pol III transcription terminator) |
| | <220> |
| | <221> misc_feature |
| | <222> (1765)..(1768) |
| | <223> 5′ overhang |
| | <220> |
| | <221> misc_recomb |
| | <222> (1775)..(1874) |
| | <223> attL2 |
| | <220> |
| | <221> primer bind |
| | <222> (1915)..(1931) |
| | <223> M13 Reverse priming site |
| | <220> |
| | <221> CDS |
| | <222> (2044)..(2853) |
| | <223> Kanamycin Resistance Gene |
| 11 | <223> Synthetic Construct |
| 12 | <223> plasmid comprisisng an entry clone vector for |
| | use in adenoviral |
| | destination vector for production of adenoviruses |
| | encoding shRNA |
| | (i.e. shSVBP-2) |
| | <220> |
| | <221> rep_origin |
| | <222> (76)..(749) |
| | <223> pUC origin |
| | <220> |
| | <221> terminator |
| | <222> (1020)..(1047) |
| | <223> rrnB T2 |
| | <220> |
| | <221> terminator |
| | <222> (1179)..(1222) |
| | <223> rrnB Tit (transcription terminator) |
| | <220> |
| | <221> primer bind |
| | <222> (1289)..(1304) |
| | <223> M13 Forward priming site |
| | <220> |
| | <221> misc_recomb |
| | <222> (1321)..(1420) |
| | <223> attL1 |
| | <220> |
| | <221> promoter |
| | <222> (1457)..(1720) |
| | <223> U6 Promotor (RNA Pol III) |
| | <220> |
| | <221> primer bind |
| | <222> (1642)..(1661) |
| | <223> U6 Forward Priming Site |
| | <220> |
| | <221> misc_feature |
| | <222> (1717)..(1720) |
| | <223> 5′ overhang |
| | <220> |
| | <221> misc_RNA |
| | <222> (1721)..(1766) |
| | <223> shSVBP2 sh2 (shRNA SVBP) |
| | <220> |
| | <221> misc_feature |
| | <222> (1742)..(1745) |
| | <223> Loop (targeting shRNA) |
| | <220> |
| | <221> terminator |
| | <222> (1767)..(1772) |
| | <223> Pol III T (Pol III transcription terminator) |
| | <220> |
| | <221> misc_feature |
| | <222> (1767)..(1770) |
| | <223> 5′ overhang |
| | <220> |
| | <221> misc_recomb |
| | <222> (1777)..(1876) |
| | <223> attL2 |
| | <220> |
| | <221> primer bind |
| | <222> (1917)..(1933) |
| | <223> M13 Reverse priming site |
| | <220> |
| | <221> CDS |
| | <222> (2046)..(2855) |
| | <223> Kanamycin resistance gene |
| 13 | <223> Synthetic Construct |
| 14 | <223> plasmid comprisisng an entry clone vector for |
| | use in adenoviral |
| | destination vector for production of adenoviruses |
| | encoding shRNA |
| | (i.e. shSVBP-3) |
| | <220> |
| | <221> rep_origin |
| | <222> (65)..(738) |
| | <223> pUC origin |
| | <220> |
| | <221> terminator |
| | <222> (1009)..(1036) |
| | <223> rrnB T2 (transcription terminator) |
| | <220> |
| | <221> terminator |
| | <222> (1168)..(1211) |
| | <223> rrnB Tit (transcription terminator) |
| | <220> |
| | <221> primer bind |
| | <222> (1278)..(1293) |
| | <223> M13 Forward priming site |
| | <220> |
| | <221> misc_recomb |
| | <222> (1310)..(1409) |
| | <223> attL1 |
| | <220> |
| | <221> promoter |
| | <222> (1446)..(1709) |
| | <223> U6 Promotor (RNA Pol III) |
| | <220> |
| | <221> primer bind |
| | <222> (1631)..(1650) |
| | <223> U6 Forward priming site |
| | <220> |
| | <221> misc_feature |
| | <222> (1706)..(1709) |
| | <223> 5′ overhnag |
| | <220> |
| | <221> misc_RNA |
| | <222> (1710)..(1755) |
| | <223> shSVBP sh3 (shRNA SVBP) |
| | <220> |
| | <221> misc_feature |
| | <222> (1731)..(1734) |
| | <223> Loop (targeting shRNA) |
| | <220> |
| | <221> terminator |
| | <222> (1756)..(1761) |
| | <223> Pol III T (Pol III transcription terminator) |
| | <220> |
| | <221> misc_feature |
| | <222> (1756)..(1759) |
| | <223> 5′ overhang |
| | <220> |
| | <221> misc_recomb |
| | <222> (1766)..(1865) |
| | <223> attL2 |
| | <220> |
| | <221> primer bind |
| | <222> (1906)..(1922) |
| | <223> M13 Reverse priming site |
| | <220> |
| | <221> CDS |
| | <222> (2035)..(2844) |
| | <223> Kanamycin Resisance Gene |
| 15 | <223> Synthetic Construct |
| 16 | <223> targeting shRNA |
| | <220> |
| | <221> misc_feature |
| | <222> (1)..(21) |
| | <223> Stem (targeting shRNA) |
| | <220> |
| | <221> misc_feature |
| | <222> (22)..(25) |
| | <223> Loop (targeting shRNA) |
| | <220> |
| | <221> misc_feature |
| | <222> (26)..(46) |
| | <223> Stem (targeting shRNA) |
| 17 | <223> targeting shRNA |
| | <220> |
| | <221> misc_feature |
| | <222> (1)..(21) |
| | <223> Stem (targeting shRNA) |
| | <220> |
| | <221> misc_feature |
| | <222> (22)..(25) |
| | <223> Loop (targeting shRNA) |
| | <220> |
| | <221> misc_feature |
| | <222> (26)..(46) |
| | <223> Stem (targeting shRNA) |
| 18 | <223> targeting shRNA |
| | <220> |
| | <221> misc_feature |
| | <222> (1)..(21) |
| | <223> Stem (targeting shRNA) |
| | <220> |
| | <221> misc_feature |
| | <222> (22)..(25) |
| | <223> Loop (targeting shRNA) |
| | <220> |
| | <221> misc_feature |
| | <222> (26)..(46) |
| | <223> Stem (targeting shRNA) |
| 19 | <223> targeting shRNA |
| | <220> |
| | <221> misc_feature |
| | <222> (1)..(21) |
| | <223> Stem (targeting shRNA) |
| | <220> |
| | <221> misc_feature |
| | <222> (22)..(25) |
| | <223> Loop (targeting shRNA) |
| | <220> |
| | <221> misc_feature |
| | <222> (26)..(46) |
| | <223> Stem (targeting shRNA) |
| 20 | <223> targeting shRNA |
| | <220> |
| | <221> misc_feature |
| | <222> (1)..(21) |
| | <223> Stem (targeting shRNA) |
| | <220> |
| | <221> misc_feature |
| | <222> (22)..(26) |
| | <223> Loop (targeting shRNA) |
| | <220> |
| | <221> misc_feature |
| | <222> (26)..(46) |
| | <223> Stem (targeting shRNA) |
| 21 | <223> targeting shRNA |
| | <220> |
| | <221> misc_feature |
| | <222> (1)..(21) |
| | <223> Stem (targeting shRNA) |
| | <220> |
| | <221> misc_feature |
| | <222> (22)..(26) |
| | <223> Loop (targeting shRNA) |
| | <220> |
| | <221> misc_feature |
| | <222> (26)..(46) |
| | <223> Stem (targeting shRNA) |
| 22 | <223> TTL |
| | <220> |
| | <221> CDS |
| | <222> (l)..(1164) |
| | <223> TTL |
| 23 | <223> Synthetic Construct |
| 24 | <220> |
| | <221> misc_feature |
| | <222> (1)..(21) |
| | <223> Stem (targeting shRNA) |
| | <220> |
| | <221> misc_feature |
| | <222> (22)..(25) |
| | <223> Loop (targeting shRNA) |
| | <220> |
| | <221> misc_feature |
| | <222> (26)..(46) |
| | <223> Stem (targeting shRNA) |
| 25 | <223> shRNA targeting VASH2 |
| | <220> |
| | <221> misc_feature |
| | <222> (1)..(21) |
| | <223> Stem (targeting shRNA) |
| | <220> |
| | <221> misc_feature |
| | <222> (22)..(25) |
| | <223> Loop (targeting shRNA) |
| | <220> |
| | <221> misc_feature |
| | <222> (26)..(46) |
| | <223> Stem (targeting shRNA) |
| 26 | <223> shRNA targeting |
| | <220> |
| | <221> misc_feature |
| | <222> (1)..(21) |
| | <223> Stem (targeting shRNA) |
| | <220> |
| | <221> misc_feature |
| | <222> (22)..(25) |
| | <223> Loop (targeting shRNA) |
| | <220> |
| | <221> misc_feature |
| | <222> (26)..(46) |
| | <223> Stem (targeting shRNA) |
| 27 | <223> TTL protein |
| 28 | <223> Inactive TTL protein (TTL E331Q) |
| | <220> |
| | <221> MUTAGEN |
| | <222> (331)..(331) |
| | <223> E331Q Mutagen (Inactive TTL) |
| 29 | <223> VP1 (AY530579)-translated |
| 30 | <223> RT/qPCR probe/primer |
| 31 | <223> RT/qPCR probe/primer |
| 32 | <223> RT/qPCR probe/primer |
| 33 | <223> RT/qPCR probe/primer |
| 34 | <223> RT/qPCR probe/primer |
| 35 | <223> RT/qPCR probe/primer |
| 36 | <223> RT/qPCR probe/primer |
| 37 | <223> RT/qPCR probe/primer |
| 38 | <223> RT/qPCR probe/primer |
| | <220> |
| | <221> misc_feature |
| | <222> (1)..(1) |
| | <223> 5′ 6-FAM modifictaion |
| | <220> |
| | <221> misc_feature |
| | <222> (9)..(9) |
| | <223> ZEN modification |
| | <220> |
| | <221> misc_feature |
| | <222> (26)..(26) |
| | <223> 3′ Iowa Black ® FQ |
| 39 | <223> RT/qPCR probe/primer |
| 40 | <223> RT/qPCR probe/primer |
| 41 | <223> RT/qPCR probe/primer |
| | <220> |
| | <221> misc_feature |
| | <222> (1)..(1) |
| | <223> 5′ 6-FAM modification |
| | <220> |
| | <221> misc_feature |
| | <222> (9)..(9) |
| | <223> ZEN modification |
| | <220> |
| | <221> misc_feature |
| | <222> (23)..(23) |
| | <223> 3′Iowa Black ® FQ |
| 42 | <223> RT/qPCR probe/primer |
| 43 | <223> RT/qPCR probe/primer |
| 44 | <223> RT/qPCR probe/primer |
| | <220> |
| | <221> misc_feature |
| | <222> (1)..(1) |
| | <223> 5′ 6-FAM modication |
| | <220> |
| | <221> misc_feature |
| | <222> (9)..(9) |
| | <223> ZEN modification |
| | <220> |
| | <221> misc_feature |
| | <222> (23)..(23) |
| | <223> 3′ Iowa Black ® FQ modicfication |
| 45 | <223> RT/qPCR probe/primer |
| 46 | <223> RT/qPCR probe/primer |
| 47 | <223> RT/qPCR probe/primer |
| 48 | <223> RT/qPCR probe/primer |
| 49 | <223> RT/qPCR probe/primer |
| 50 | <223> RT/qPCR probe/primer |
| 51 | <223> RT/qPCR probe/primer |
| 52 | <223> RT/qPCR probe/primer |
| 52 | <223> RT/qPCR probe/primer |
| 54 | <223> RT/qPCR probe/primer |
| 55 | <223> peptide for immunization for antibody production |
| 56 | <223> VASH1 CDS |
| | <220> |
| | <221> misc_RNA |
| | <222> (1747)..(1767) |
| | <223> VASHI sh1 target |
| | <220> |
| | <221> misc_RNA |
| | <222> (1766)..(1786) |
| | <223> VASHI sh2 target |
| | <220> |
| | <221> misc_RNA |
| | <222> (1879)..(1899) |
| | <223> VASH1 sh3 target |
| 58 | <223> VASH2 CDS |
| | <220> |
| | <221> misc_RNA |
| | <222> (999)..(1019) |
| | <223> VASH2 sh1 target |
| | <220> |
| | <221> misc_RNA |
| | <222> (1007)..(1027) |
| | <223> VASH2 sh2 target |
| | <220> |
| | <221> misc_RNA |
| | <222> (1008)..(1028) |
| | <223> VASH2 sh3 target |
| 60 | <223> SVBPCDS |
| | <220> |
| | <221> misc_RNA |
| | <222> (345)..(365) |
| | <223> SVBP sh1 target |
| | <220> |
| | <221> misc_RNA |
| | <222> (394)..(414) |
| | <223> SVBP sh2 target |
| | <220> |
| | <221> misc_RNA |
| | <222> (397)..(417) |
| | <223> SVBP sh3 target |
| 62 | <223> vector genome of AAV.cTNT.TTL-IRES-dsRED |
| | <220> |
| | <221> repeat_region |
| | <222> (l)..(130) |
| | <223> 5′ITR |
| | <220> |
| | <221> promoter |
| | <222> (214)..(655) |
| | <223> cTNT promoter |
| | <220> |
| | <221> Intron |
| | <222> (772)..(904) |
| | <223> chimeric intron |
| | <220> |
| | <221> misc_feature |
| | <222> (997)..(2160) |
| | <223> TTL |
| | <220> |
| | <221> RBS |
| | <222> (2194)..(2778) |
| | <223> IRES |
| | <220> |
| | <221> misc_feature |
| | <222> (2782)..(3459) |
| | <223> DsRED |
| | <220> |
| | <221> misc_feature |
| | <222> (3523)..(4110) |
| | <223> WRPE |
| | <220> |
| | <221> polyA_signal |
| | <222> (4132)..(4258) |
| | <223> Rabbit globin polyA |
| | <220> |
| | <221> repeat_region |
| | <222> (4347)..(4476) |
| | <223> 3′ITR |
| 63 | <223> expression cassette AAV.cTNT.TTL-IRES- |
| | dsRED |
| | <220> |
| | <221> promoter |
| | <222> (1)..(442) |
| | <223> cTNT promoter |
| | <220> |
| | <221> Intron |
| | <222> (559)..(691) |
| | <223> chimeric intron |
| | <220> |
| | <221> misc_feature |
| | <222> (784)..(1947) |
| | <223> TTL |
| | <220> |
| | <221> RBS |
| | <222> (1981)..(2565) |
| | <223> IRES |
| | <220> |
| | <221> misc_feature |
| | <222> (2569)..(3246) |
| | <223> DsRed |
| | <220> |
| | <221> misc_feature |
| | <222> (3310)..(3897) |
| | <223> WRPE |
| | <220> |
| | <221> polyA_signal |
| | <222> (3919)..(4045) |
| | <223> Rabbit globin polyA |

1. A replication-defective vector useful for treating patients having heart failure comprising an expression cassette which comprises a coding sequence for an inhibitor of vasohibin (VASH)-small vasohibin binding protein (SVBP) complex operably linked to regulatory sequences which direct expression thereof.

2. The replication-defective vector according to claim 1, wherein the inhibitor is an RNA specifically targeted to VASH1, VASH2 or SVBP.

3. The replication-defective vector according to claim 1, wherein the inhibitor is an RNA specifically targeted to VASH1.

4. The replication-defective vector according to claim 2, wherein the RNA is a short hairpin RNA (shRNA).

5. The replication-defective vector according to claim 2, wherein the inhibitor is an RNA specifically targeted to:
(a)
sh1:
(Nucleotide 1-21 of SEQ ID NO: 16)
gctgcagtacaatcacacagg,
sh2:
(Nucleotide 1-21 of SEQ ID NO: 17)
gggacacagttctttgaaatt,
or
sh3:
(Nucleotide 1-21 of SEQ ID NO: 18)
gggaatttacctcaccaacag
for VASH1;
(b)
sh1:
(Nucleotide 1-21 SEQ ID NO: 24)
gtcaagaaggtcaagattggg,
sh2:
(Nucleotide 1-21 SEQ ID NO: 25)
ggtcaagattgggctgtacgt,
sh3:
(Nucleotide 1-21 SEQ ID NO: 26)
gtcaagattgggctgtacgt[[,]]
for VASH2;
or
(c)
sh1:
(Nucleotide 1-21 of SEQ ID NO: 19)
gacaaagagcagagatctatg,
sh2:
(Nucleotide 1-21 of SEQ ID NO: 20)
gcagcagcagtttgatgagtt,
sh3:
(Nucleotide 1-21 of SEQ ID NO: 21)
gcagcagtttgatgagttctg[[]]
for SVBP.

6. The replication-defective vector according to claim 1, wherein the inhibitor is an shRNA having the sequence of:
(a) (VASH1 sh1)
(SEQ ID NO: 16)
gctgcagtacaatcacacaggcgaacctgtgtgattgtactgcagc;
(b) (VASH1 sh2)
(SEQ ID NO: 17)
gggacacagttctttgaaattcgaaaatttcaaagaactgtgtccc;
or
(c) (VASH1 sh3)
(SEQ ID NO: 18)
gggaatttacctcaccaacagcgaactgttggtgaggtaaattccc.

7. The replication-defective vector according to claim 6, wherein the inhibitor is an shRNA having a mismatch in stem region of 1, 2, 3, or 4 nucleotide/s in the sequences of (a), (b) or (c).

8. The replication-defective vector according to claim 6, wherein the inhibitor is an shRNA further comprises the loop sequence of:
a)
caaga;
or
b)
tcga.

9. The replication-defective vector according to claim 1, wherein the vector further comprises coding sequences for at least a second VASH-SVBP inhibitor operably linked to regulatory sequences which control expression thereof.

10. The replication-defective vector according to claim 1, wherein the vector further comprises coding sequences for tubulin tyrosine ligase (TTL) operably linked to regulatory sequences which control expression thereof.

11. The replication-defective vector according to claim 10, wherein the vector further comprises coding sequence for additional shRNA operably linked to regulatory sequences which control expression thereof in a bicistronic expression cassette.

12. The replication-defective vector according to claim 1, wherein the regulatory sequences comprise a regulatable or an inducible promoter for controlling expression of the inhibitor.

13. The replication-defective vector according to claim 1, wherein the replication-defective vector is a replication-defective viral vector selected from a lentivirus vector or an adeno-associated viral vector.

14. The replication-defective vector according to claim 1, wherein the replication-defective vector is an adeno-associated viral vector.

15. The replication-defective vector according to claim 14, wherein the replication-defective adeno-associated viral vector has an adeno-associated virus capsid from clade F.

16. The replication-defective vector according to claim 1, wherein the replication-defective virus has an adeno-associated virus capsid from AAV9, AAVhu31, AAVhu32, AAVhu68, or a variant of any one of these adeno-associated virus capsids which targets cardiac cells.

17. The replication-defective vector according to claim 1, wherein the vector is a non-viral vector.

18. A pharmaceutical composition comprising a replication-defective vector according to claim 1 and a diluent, suspending agent, and/or optional preservative.

19-24. (canceled)

25. A method for treating failing human cardiomyocytes comprising delivering to a patient a replication-defective vector according to claim which comprises an expression cassette which comprises a coding sequence for an inhibitor of vasohibin (VASH)-small vasohibin binding protein (SVBP) complex operably linked to regulatory sequences which direct expression thereof.

26. The method according to claim 25, wherein the patient has heart failure with reduced ejection fraction (HFrEF).

27. The method according to claim 25, wherein the patient has heart failure with preserved ejection function (HFpEF).

28. The method according to claim 25, further comprising co-administering the replication-defective vector in a combination therapy with a calcium desensitizer, myosin inhibitors, a small molecule VASH inhibitor, a small molecule small vasohibin binding protein inhibitor, tubulin tyrosine ligase, and/or parthenolide therapy.


SEQUENCE LISTING (63 sequences)

SEQ ID NO: 1 - DNA, 7297 bp, Artificial Sequence
  plasmid comprising AAV vector genome for expression of TTL
  repeat_region (1)..(130): 5′UTR
  promoter (214)..(655): cTNT cardiac specific promoter
  Intron (772)..(904): Intron 1
  CDS (997)..(2160): TTL cds
  RBS (2194)..(2778): IRES
  CDS (2782)..(3459): dsRED
  misc_feature (3523)..(4110): WPRE (Woodchuck Hepatitis Virus Posttranscriptional Regulatory Element)
  misc_feature (4132)..(4258): Rabbit globin poly A
  repeat_region (4347)..(4476): 3′ITR
  misc_feature (4653)..(5108): f1 ori
  misc_feature (5239)..(6096): AP(R)
  rep_origin (6270)..(6858): Origin of replication
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttacgggccccccctcgaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgctaccggactcagatctggtaccatgtacccatacgacgtcccagactacgctatgtacaccttcgtggtacgcgatgagaacagcagcgtctacgccgaggtctcccggctgctcctcgccaccggccactggaagaggctgcggcgagacaaccccagattcaacctgatgctgggagagaggaatcggctgcccttcgggagactgggtcacgagcccgggctggtacagttggtgaattactacaggggtgctgacaaactgtgtcgcaaagcttctttagtgaagctaatcaagacaagccctgaactggctgagtcctgcacatggttccctgaatcttatgtgatttatccaaccaatctcaagactccagttgctccagcacagaatggaattcagccaccaatcagtaactcaaggacagatgaaagagaattctttctcgcctcttataacagaaagaaagaggatggagagggcaacgtttggattgcaaagtcatcagccggtgccaaaggtgaaggcattctcatctcctcagaggcttcagagcttctcgatttcatagacaaccagggccaagtgcacgtgatccagaaatatcttgagcaccctctgctgcttgagccaggtcatcgcaagtttgacatccgaagctgggtcttggtggatcatcagtataatatctacctctatagagagggtgtgcttcggactgcttcagaaccatatcatgttgataatttccaagacaaaacctgccatttgaccaatcactgcattcaaaaagagtattcaaagaactacgggaagtatgaagaaggaaatgaaatgttcttcaaggagttcaatcagtacctaacaagtgctttgaacattaccctagaaagtagtatcttactacaaatcaaacatataataaggaactgcctcctgagcgtggagcctgccattagcaccaagcacctcccttaccagagcttccagctcttcggctttgacttcatggtcgatgaggagctgaaggtgtggctcattgaggtcaacggtgcccctgcatgtgctcagaagctctatgcagaactgtgccaaggcatcgtggacatagccatttccagtgtcttcccacccccagatgtggagcaacctcagacccagccagctgccttcatcaagctgtgactcgagcctaagcttctagataagatgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtctatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcctcgacccgggcggcctcgaggacggggtgaactacgcctgaatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgacgtgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 2 - protein, 387 aa, Artificial Sequence
  Synthetic Construct
MYPYDVPDYAMYTFVVRDENSSVYAEVSRLLLATGHWKRLRRDNPRFNLMLGERNRLPFGRLGHEPGLVQLVNYYRGADKLCRKASLVKLIKTSPELAESCTWFPESYVIYPTNLKTPVAPAQNGIQPPISNSRTDEREFFLASYNRKKEDGEGNVWIAKSSAGAKGEGILISSEASELLDFIDNQGQVHVIQKYLEHPLLLEPGHRKFDIRSWVLVDHQYNIYLYREGVLRTASEPYHVDNFQDKTCHLTNHCIQKEYSKNYGKYEEGNEMFFKEFNQYLTSALNITLESSILLQIKHIIRNCLLSVEPAISTKHLPYQSFQLFGFDFMVDEELKVWLIEVNGAPACAQKLYAELCQGIVDIAISSVFPPPDVEQPQTQPAAFIKL

SEQ ID NO: 3 - protein, 225 aa, Artificial Sequence
  Synthetic Construct
MASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFL

SEQ ID NO: 4 - DNA, 2900 bp, Artificial Sequence
  plasmid comprisisng an entry clone vector for use in adenoviral destination vector for production of adenoviruses encoding shRNA (i.e. shVASH1-1)
  rep_origin (87)..(760): pUC origin
  terminator (1031)..(1058): rrnB T2 transcription terminator
  terminator (1190)..(1233): rrnB T1 t transcription terminator
  primer_bind (1300)..(1315): M13 Forward priming site
  misc_recomb (1332)..(1431): aatL1
  promoter (1468)..(1731): U6 Promotor (RNA Pol III)
  primer_bind (1653)..(1672): U6 Forward priming site
  misc_feature (1728)..(1731): shRNA 5′ overhang
  misc_RNA (1732)..(1777): shVASH1 sh1 (shRNA)
  misc_feature (1732)..(1752): Stem (targeting shRNA)
  misc_feature (1753)..(1756): Loop (targeting shRNA)
  misc_feature (1757)..(1777): Stem (targeting shRNA)
  terminator (1778)..(1783): Pol III T(Pol III Transcription terminator)
  misc_feature (1778)..(1781): 5′ overhang (targeting shRNA)
  misc_recomb (1788)..(1887): attl2
  primer_bind (1928)..(1944): M13 Reverse Priming Site
  CDS (2057)..(2866): Kan (Kanamycin Resistance Gene)
gcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgcagtacaatcacacaggcgaacctgtgtgattgtactgcagcttttttctagacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcaga

SEQ ID NO: 5 - protein, 269 aa, Artificial Sequence
  Synthetic Construct
MSHIQRETSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 6 - DNA, 2900 bp, Artificial Sequence
  plasmid comprisisng an entry clone vector for use in adenoviral destination vector for production of adenoviruses encoding shRNA (i.e. shVASH1-2)
  rep_origin (90)..(763): pUC ori
  terminator (1034)..(1061): rrnB T2 (transcription terminator)
  terminator (1193)..(1236): rrnR T1 t (trascription terminator)
  protein_bind (1303)..(1318): M13 Forward priming cite
  misc_recomb (1335)..(1434): attL1
  promoter (1471)..(1734): U6 promotor (RNA pol III)
  primer_bind (1656)..(1675): U6 Forward priming site
  misc_feature (1731)..(1734): 5′ overhang
  misc_RNA (1735)..(1780): shVASH1 sh2 (shRNA)
  misc_feature (1735)..(1755): Stem (targeting shRNA)
  misc_feature (1756)..(1759): Loop (targeting shRNA)
  misc_feature (1760)..(1780): Stem (targeting shRNA)
  terminator (1781)..(1786): Pol III T (Pol III Transcription Terminator)
  misc_feature (1781)..(1784): 5′ overhang
  misc_recomb (1791)..(1890): attL2
  primer_bind (1931)..(1947): M13 Reverse priming site
  CDS (2060)..(2869): KAnamycin Resistance Gene
agagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggacacagttctttgaaattcgaaaatttcaaagaactgtgtcccttttttctagacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggc

SEQ ID NO: 7 - protein, 269 aa, Artificial Sequence; identical to SEQ ID NO: 5
  Synthetic Construct

SEQ ID NO: 8 - DNA, 2900 bp, Artificial Sequence
  plasmid comprisisng an entry clone vector for use in adenoviral destination vector for production of adenoviruses encoding shRNA (i.e. shVASH1-3)
  rep_origin (63)..(736): pUC origin
  terminator (1007)..(1034): rrnB T2 transcription terminator
  terminator (1166)..(1209): rrnB T1 t transcription temrinator
  primer_bind (1276)..(1291): M13 Forward primer bind
  misc_recomb (1308)..(1407): attL1
  promoter (1444)..(1707): U6 Promotor (RNA Pol III)
  primer_bind (1629)..(1648): U6 Forward priming site
  misc_feature (1704)..(1707): 5′ overhang
  misc_RNA (1708)..(1753): shVASH1 sh3 (shRNA)
  misc_feature (1708)..(1728): Stem (targeting shRNA)
  misc_feature (1729)..(1732): Loop (targeting shRNA)
  misc_feature (1733)..(1753): Stem (targeting shRNA)
  terminator (1754)..(1759): Pol III T (Pol III Transcription Terminator)
  misc_feature (1754)..(1757): 5′ overhang
  misc_recomb (1764)..(1863): attL2
  primer_bind (1904)..(1920): M13 reverse priming site
  CDS (2033)..(2842): Kanamycin Resistance Gene
gcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaatttacctcaccaacagcgaactgttggtgaggtaaattcccttttttctagacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacg

SEQ ID NO: 9 - protein, 269 aa, Artificial Sequence; identical to SEQ ID NO: 5
  Synthetic Construct

SEQ ID NO: 10 - DNA, 2900 bp, Artificial Sequence
  plasmid comprisisng an entry clone vector for use in adenoviral destination vector for production of adenoviruses encoding shRNA (i.e. shSVBP-1)
  rep_origin (74)..(747): pUC origin
  terminator (1018)..(1045): rrnB T2 transcription terminator
  terminator (1177)..(1220): rrnB T1 t transcription terminator
  primer_bind (1287)..(1302): M13 Forward priming site
  misc_recomb (1319)..(1418): attL1
  promoter (1455)..(1718): U 6 Promotor (RNA Pol III)
  primer_bind (1640)..(1659): U6 Priming site
  misc_feature (1715)..(1718): 5′ overhang
  misc_RNA (1719)..(1764): shSVBP sh 1  (shRNA SVBP1)
  misc_feature (1740)..(1743): Loop (targeting shRNA)
  terminator (1765)..(1770): Pol III T (Pol III transcription terminator)
  misc_feature (1765)..(1768): 5′ overhang
  misc_recomb (1775)..(1874): attL2
  primer_bind (1915)..(1931): M13 Reverse priming site
  CDS (2044)..(2853): Kanamycin Resistance Gene
ttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaaagagcagagatctatgcgaacatagatctctgctctttgtcttttttctagacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgac

SEQ ID NO: 11 - protein, 269 aa, Artificial Sequence; identical to SEQ ID NO: 5
  Synthetic Construct

SEQ ID NO: 12 - DNA, 2900 bp, Artificial Sequence
  plasmid comprisisng an entry clone vector for use in adenoviral destination vector for production of adenoviruses encoding shRNA (i.e. shSVBP-2)
  rep_origin (76)..(749): pUC origin
  terminator (1020)..(1047): rrnB T2
  terminator (1179)..(1222): rrnB T1 t (transcription terminator)
  primer_bind (1289)..(1304): M13 Forward priming site
  misc_recomb (1321)..(1420): attL1
  promoter (1457)..(1720): U6 Promotor (RNA Pol III)
  primer_bind (1642)..(1661): U6 Forward Priming Site
  misc_feature (1717)..(1720): 5′ overhang
  misc_RNA (1721)..(1766): shSVBP2 sh2 (shRNA SVBP)
  misc_feature (1742)..(1745): Loop (targeting shRNA)
  terminator (1767)..(1772): Pol III T (Pol III transcription terminator)
  misc_feature (1767)..(1770): 5′ overhang
  misc_recomb (1777)..(1876): attL2
  primer_bind (1917)..(1933): M13 Reverse priming site
  CDS (2046)..(2855): Kanamycin resistance gene
acttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcagcagcagtttgatgagttcgaaaactcatcaaactgctgctgcttttttctagacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctg

SEQ ID NO: 13 - protein, 269 aa, Artificial Sequence; identical to SEQ ID NO: 5
  Synthetic Construct

SEQ ID NO: 14 - DNA, 2900 bp, Artificial Sequence
  plasmid comprisisng an entry clone vector for use in adenoviral destination vector for production of adenoviruses encoding shRNA (i.e. shSVBP-3)
  rep_origin (65)..(738): pUC origin
  terminator (1009)..(1036): rrnB T2 (transcription terminator)
  terminator (1168)..(1211): rrnB T1 t (transcription terminator)
  primer_bind (1278)..(1293): M13 Forward priming site
  misc_recomb (1310)..(1409): attL1
  promoter (1446)..(1709): U6 Promotor (RNA Pol III)
  primer_bind (1631)..(1650): U6 Forward priming site
  misc_feature (1706)..(1709): 5′ overhnag
  misc_RNA (1710)..(1755): shSVBP sh3 (shRNA SVBP)
  misc_feature (1731)..(1734): Loop (targeting shRNA)
  terminator (1756)..(1761): Pol III T (Pol III transcription terminator)
  misc_feature (1756)..(1759): 5′ overhang
  misc_recomb (1766)..(1865): attL2
  primer_bind (1906)..(1922): M13 Reverse priming site
  CDS (2035)..(2844): Kanamycin Resisance Gene
cggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcagcagtttgatgagttctgcgaacagaactcatcaaactgctgcttttttctagacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacggga

SEQ ID NO: 15 - protein, 269 aa, Artificial Sequence; identical to SEQ ID NO: 5
  Synthetic Construct

SEQ ID NO: 16 - DNA, 46 bp, Artificial Sequence
  targeting shRNA
  misc_feature (1)..(21): Stem (targeting shRNA)
  misc_feature (22)..(25): Loop (targeting shRNA)
  misc_feature (26)..(46): Stem (targeting shRNA)
gctgcagtacaatcacacaggcgaacctgtgtgattgtactgcagc

SEQ ID NO: 17 - DNA, 46 bp, Artificial Sequence
  targeting shRNA
  misc_feature (1)..(21): Stem (targeting shRNA)
  misc_feature (22)..(25): Loop (targeting shRNA)
  misc_feature (26)..(46): Stem (targeting shRNA)
gggacacagttctttgaaattcgaaaatttcaaagaactgtgtccc

SEQ ID NO: 18 - DNA, 46 bp, Artificial Sequence
  targeting shRNA
  misc_feature (1)..(21): Stem (targeting shRNA)
  misc_feature (22)..(25): Loop (targeting shRNA)
  misc_feature (26)..(46): Stem (targeting shRNA)
gggaatttacctcaccaacagcgaactgttggtgaggtaaattccc

SEQ ID NO: 19 - DNA, 46 bp, Artificial Sequence
  targeting shRNA
  misc_feature (1)..(21): Stem (targeting shRNA)
  misc_feature (22)..(25): Loop (targeting shRNA)
  misc_feature (26)..(46): Stem (targeting shRNA)
gacaaagagcagagatctatgcgaacatagatctctgctctttgtc

SEQ ID NO: 20 - DNA, 46 bp, Artificial Sequence
  targeting shRNA
  misc_feature (1)..(21): Stem (targeting shRNA)
  misc_feature (22)..(26): Loop (targeting shRNA)
  misc_feature (26)..(46): Stem (targeting shRNA)
gcagcagcagtttgatgagttcgaaaactcatcaaactgctgctgc

SEQ ID NO: 21 - DNA, 46 bp, Artificial Sequence
  targeting shRNA
  misc_feature (1)..(21): Stem (targeting shRNA)
  misc_feature (22)..(26): Loop (targeting shRNA)
  misc_feature (26)..(46): Stem (targeting shRNA)
gcagcagtttgatgagttctgcgaacagaactcatcaaactgctgc

SEQ ID NO: 22 - DNA, 1164 bp, Artificial Sequence
  TTL
  CDS (1)..(1164): TTL
atgtacccatacgacgtcccagactacgctatgtacaccttcgtggtacgcgatgagaacagcagcgtctacgccgaggtctcccggctgctcctcgccaccggccactggaagaggctgcggcgagacaaccccagattcaacctgatgctgggagagaggaatcggctgcccttcgggagactgggtcacgagcccgggctggtacagttggtgaattactacaggggtgctgacaaactgtgtcgcaaagcttctttagtgaagctaatcaagacaagccctgaactggctgagtcctgcacatggttccctgaatcttatgtgatttatccaaccaatctcaagactccagttgctccagcacagaatggaattcagccaccaatcagtaactcaaggacagatgaaagagaattctttctcgcctcttataacagaaagaaagaggatggagagggcaacgtttggattgcaaagtcatcagccggtgccaaaggtgaaggcattctcatctcctcagaggcttcagagcttctcgatttcatagacaaccagggccaagtgcacgtgatccagaaatatcttgagcaccctctgctgcttgagccaggtcatcgcaagtttgacatccgaagctgggtcttggtggatcatcagtataatatctacctctatagagagggtgtgcttcggactgcttcagaaccatatcatgttgataatttccaagacaaaacctgccatttgaccaatcactgcattcaaaaagagtattcaaagaactacgggaagtatgaagaaggaaatgaaatgttcttcaaggagttcaatcagtacctaacaagtgctttgaacattaccctagaaagtagtatcttactacaaatcaaacatataataaggaactgcctcctgagcgtggagcctgccattagcaccaagcacctcccttaccagagcttccagctcttcggctttgacttcatggtcgatgaggagctgaaggtgtggctcattgaggtcaacggtgcccctgcatgtgctcagaagctctatgcagaactgtgccaaggcatcgtggacatagccatttccagtgtcttcccacccccagatgtggagcaacctcagacccagccagctgccttcatcaagctgtga

SEQ ID NO: 23 - protein, 387 aa, Artificial Sequence; identical to SEQ ID NO: 2
  Synthetic Construct

SEQ ID NO: 24 - DNA, 46 bp, Artificial Sequence
  shRNA targeting VASH2
  misc_feature (1)..(21): Stem (targeting shRNA)
  misc_feature (22)..(25): Loop  (targeting shRNA)
  misc_feature (26)..(46): Stem (targeting shRNA)
gtcaagaaggtcaagattgggcgaacccaatcttgaccttcttgac

SEQ ID NO: 25 - DNA, 46 bp, Artificial Sequence
  shRNA targeting VASH2
  misc_feature (1)..(21): Stem (targeting shRNA)
  misc_feature (22)..(25): Loop (targeting shRNA)
  misc_feature (26)..(46): Stem (targeting shRNA)
ggtcaagattgggctgtacgtcgaaacgtacagcccaatcttgacc

SEQ ID NO: 26 - DNA, 46 bp, Artificial Sequence
  shRNA targeting
  misc_feature (1)..(21): Stem (targeting shRNA)
  misc_feature (22)..(25): Loop (targeting shRNA)
  misc_feature (26)..(46): Stem (targeting shRNA)
gtcaagattgggctgtacgtccgaagacgtacagcccaatcttgac

SEQ ID NO: 27 - protein, 377 aa, Artificial Sequence
  TTL protein
MYTFVVRDENSSVYAEVSRLLLATGHWKRLRRDNPRFNLMLGERNRLPFGRLGHEPGLVQLVNYYRGADKLCRKASLVKLIKTSPELAESCTWFPESYVIYPTNLKTPVAPAQNGIQPPISNSRTDEREFFLASYNRKKEDGEGNVWIAKSSAGAKGEGILISSEASELLDFIDNQGQVHVIQKYLEHPLLLEPGHRKFDIRSWVLVDHQYNIYLYREGVLRTASEPYHVDNFQDKTCHLTNHCIQKEYSKNYGKYEEGNEMFFKEFNQYLTSALNITLESSILLQIKHIIRNCLLSVEPAISTKHLPYQSFQLFGFDFMVDEELKVWLIEVNGAPACAQKLYAELCQGIVDIAISSVFPPPDVEQPQTQPAAFIKL

SEQ ID NO: 28 - protein, 377 aa, Artificial Sequence
  Inactive TTL protein (TTL E331Q)
  MUTAGEN (331)..(331): E331Q Mutagen (Inactive TTL)
MYTFVVRDENSSVYAEVSRLLLATGHWKRLRRDNPRFNLMLGERNRLPFGRLGHEPGLVQLVNYYRGADKLCRKASLVKLIKTSPELAESCTWFPESYVIYPTNLKTPVAPAQNGIQPPISNSRTDEREFFLASYNRKKEDGEGNVWIAKSSAGAKGEGILISSEASELLDFIDNQGQVHVIQKYLEHPLLLEPGHRKFDIRSWVLVDHQYNIYLYREGVLRTASEPYHVDNFQDKTCHLTNHCIQKEYSKNYGKYEEGNEMFFKEFNQYLTSALNITLESSILLQIKHIIRNCLLSVEPAISTKHLPYQSFQLFGFDFMVDEELKVWLIQVNGAPACAQKLYAELCQGIVDIAISSVFPPPDVEQPQTQPAAFIKL

SEQ ID NO: 29 - DNA, 2211 bp, Adeno-associated virus 9
  CDS (1)..(2211): VP1 (AY530579)- translated
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 30 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 736 aa, Adeno-associated virus 9; identical to SEQ ID NO: 30

SEQ ID NO: 32 - DNA, 18 bp, Artificial Sequence
  RT/qPCR probe/primer
cgtgccgcctggagaaac

SEQ ID NO: 33 - DNA, 21 bp, Artificial Sequence
  RT/qPCR probe/primer
tgggagttgctgttgaagtcg

SEQ ID NO: 34 - DNA, 22 bp, Artificial Sequence
  RT/qPCR probe/primer
tcgtcggctggaaagtaggcac

SEQ ID NO: 35 - DNA, 22 bp, Artificial Sequence
  RT/qPCR probe/primer
tcgtcggctggaaagtaggcac

SEQ ID NO: 36 - DNA, 24 bp, Artificial Sequence
  RT/qPCR probe/primer
aaccagccttcagagtggagaagg

SEQ ID NO: 37 - DNA, 25 bp, Artificial Sequence
  RT/qPCR probe/primer
gctccgtcatgactctgttgagagc

SEQ ID NO: 38 - DNA, 26 bp, Artificial Sequence
  RT/qPCR probe/primer
  misc_feature (1)..(1): 5′ 6-FAM modifictaion
  misc_feature (9)..(9): ZEN modification
  misc_feature (26)..(26): 3′  Iowa Black FQ
tcaagatcttcatccgcatgtccctg

SEQ ID NO: 39 - DNA, 22 bp, Artificial Sequence
  RT/qPCR probe/primer
gaagcaacttgtcctcaatgtc

SEQ ID NO: 40 - DNA, 21 bp, Artificial Sequence
  RT/qPCR probe/primer
ggattctcacttgggttggag

SEQ ID NO: 41 - DNA, 23 bp, Artificial Sequence
  RT/qPCR probe/primer
  misc_feature (1)..(1): 5′ 6-FAM modification
  misc_feature (9)..(9): ZEN modification
  misc_feature (23)..(23): 3′ Iowa Black FQ
tgcctactttccagccgacaacg

SEQ ID NO: 42 - DNA, 20 bp, Artificial Sequence
  RT/qPCR probe/primer
gcccaagattcccataccaa

SEQ ID NO: 43 - DNA, 20 bp, Artificial Sequence
  RT/qPCR probe/primer
actgtgtccctgtgtgattg

SEQ ID NO: 44 - DNA, 23 bp, Artificial Sequence
  RT/qPCR probe/primer
  misc_feature (1)..(1): 5′ 6-FAM modication
  misc_feature (9)..(9): ZEN modification
  misc_feature (23)..(23): 3′ Iowa Black FQ modicfication
cagcaccagcatcaccccatttg

SEQ ID NO: 45 - DNA, 20 bp, Artificial Sequence
  RT/qPCR probe/primer
aacccatcaccatcttccag

SEQ ID NO: 46 - DNA, 21 bp, Artificial Sequence
  RT/qPCR probe/primer
ccagtagactccacgacatac

SEQ ID NO: 47 - DNA, 20 bp, Artificial Sequence
  RT/qPCR probe/primer
atgttcgtcatgggtgtgaa

SEQ ID NO: 48 - DNA, 20 bp, Artificial Sequence
  RT/qPCR probe/primer
tgagtccttccacgatacca

SEQ ID NO: 49 - DNA, 20 bp, Artificial Sequence
  RT/qPCR probe/primer
agaggaaggggaagaggacc

SEQ ID NO: 50 - DNA, 20 bp, Artificial Sequence
  RT/qPCR probe/primer
gtaggcacactcggtatggg

SEQ ID NO: 51 - DNA, 20 bp, Artificial Sequence
  RT/qPCR probe/primer
gttccacgtcaacaagagcg

SEQ ID NO: 52 - DNA, 20 bp, Artificial Sequence
  RT/qPCR probe/primer
cgacagcctgtagtttggga

SEQ ID NO: 53 - DNA, 20 bp, Artificial Sequence
  RT/qPCR probe/primer
cagcagagttgagaaggcca

SEQ ID NO: 54 - DNA, 20 bp, Artificial Sequence
  RT/qPCR probe/primer
ccaggaggctgcatctgttt

SEQ ID NO: 55 - protein, 25 aa, Artificial Sequence
  peptide for immunization for antibody production
RIRGATDLPKIPIPSVPTFQPTTPV

SEQ ID NO: 56 - DNA, 6449 bp, Homo sapiens
  CDS (1355)..(2449): VASH1 CDS
  misc_RNA (1747)..(1767): VASH1 sh1 target
  misc_RNA (1766)..(1786): VASH1 sh2 target
  misc_RNA (1879)..(1899): VASH1 sh3 target
gcgggccgtagtcgagcctggtgggcggctggctggcaggggacgtggtcggcggcagcggggcccaggggcggcggggctgcagcagcggcggccccagcttcgccgagcagcgatcggctggtgggcttctcgttggcgggctccgcgttggcgggctgctccccaggcaccagcgcagcgcgcgctcgcccggcttcgtcactcgcctccagctacatccgccggccgaggctgcgcgagcctccgggcttgcggtccccgccccgcggtccttcctccaccctcagccagggcagctgcagcccgagcgaacagccacggagcatcctccgcccgcccgggccgcgccaccgcccatgctgcagccagtcccaggcagcgaccccgcggccgcagcccgccgctcagtgacctcagcctggcccctgcgcgccgcccgagccggtcccgctgagccgcgggccccgtgccctgcgatggctcggctggtgcagcgcggcgccaggtgccagccgtcctcccgctgagacgcgcccgagtggggacccgctgggcctcggggctcgcagccttcgcctccccgccgcgcccgctccctttctggggactccgccgctgtttctggggacgaggggacaggggacccagacaaagcccactttgtgcagggagttggccgcaggcggggaatgtgcgcgtcggcgcgcgccccctccccgctcccggccagctgcgagtcttggctcccggacttgtctcgtcgcgtcggagaaatcgccccccagcgccgctctcccgcccgggggtcttggttccgagctcgcgcggccgggagtcgcctcggtcttccttggggcgcgcgcagatgtgagcgtgcgagagttgtgtaggggattttgttccctccgaaactgagacccagggcgcccagtgggcacccgtgccttgactctgtcctttctgcagccgctggtccgagctgtctggcctcagtttccctccgacttttctccgctctgccagccctcactgctgcccgtcattgttctcgcagttagatgggggtgctttgtgacggctgccaagttggggtgtgttctctttattccgtttttcaaacagaacaaggcctccaaggctgaccccagacaacccaccccctcggaccctaattcaccttattgcactgattttttttatcaagtcgtattttattgtacaggagccacgccctgatttcttaaaggcgccttgcactctggccatgtgttatctctgcagccggtgtgtgggaggcctcttgtgagccagttgttttcccgcctccaccacccccctcgaagatttagggatgccaggggggaagaaggtggctgggggtggcagcagcggtgccactccaacgtccgctgcggccaccgccccctctggggtcaggcgtttggagaccagcgaaggaacctcagcccagagagatgaggagccagaagaggaaggggaagaggacctgcgagacggaggcgtccccttctttgtcaaccggggtgggctacctgtggatgaggccacctgggaaaggatgtggaaacacgtggccaagatccaccccgatggagagaaggtggcgcaacggatccgtggggccacagacctgcccaagatccccataccgagtgtgcctacgttccagccgtctacacctgtccctgagcgcctggaagctgtgcagcgctacatcagagagctgcagtacaatcacacagggacacagttctttgaaattaagaagagcagacctctgacagggctgatggacctggccaaggaaatgaccaaagaggccctgccaatcaaatgcctggaagccgtgatcctgggaatttacctcaccaacagcatgcccaccctggagcgcttccccatcagcttcaagacctacttctcagggaactacttccgccacatcgtgctgggggtgaacttcgcgggccgctacggtgcgctgggcatgagtcggcgcgaggacctgatgtacaagccgcccgccttccgcacgctcagcgagctcgtgctggacttcgaggccgcctacggccgctgctggcacgtgctcaagaaggtgaagctgggccagagcgtgtcacacgacccgcacagcgtggagcagatcgagtggaagcactcggtgctggacgtggagcgcctgggccgcgatgacttccgcaaggagctggagcgccacgcccgcgacatgcggctcaagattggcaaagggacgggccctccctctcccaccaaggaccggaagaaggatgtttcttccccgcagcgggcccagtccagcccccaccgcaggaacagccgcagtgaaagacggccctcgggtgacaagaagacttccgagcccaaagccatgccagaccttaacgggtaccagatccgggtctgaggcggatgccagcaccccaggccccacccactcttgggggccaggatccacctgctggaaccagccttatgcatggggaaggcggggctggtgacaaggcagggcaagaggctgcaggaagagtgtgttccagctcagccccccaagctgctctcgctcccactgagccaagccccctaactttgggcctagaggccgttagtattttatttggagtttttaactctacaactgaagtttaaggtatttggggaaaacttagtccaaatggatctgctgatggtgggaaggccagtgcttaacaaatccatgtgtcatggggccaggtgagggaaactgctggttctgctggtgcctctgcccctggcttctctctgggagttgggtgcatcttatcagtgggaaatctcccagccttaccaggcctgtgatggggggtgggggtggggtggagatgtttctccagttctgcctgccctggcagaatcttgacccagggaaagggaagcagggtaggagtccttctgagaaaggtctgtgtagcccattaaccaggagcttggcacaggccacatctgccccaagagcatgagctcgtggctcaggagagccttcaggcccttgaggcccccatgggcagtgctgtgtgggcagaggaggggtgatatgagagcgagcccagggaaggacctctgggcaaaaagttcccaggccctaactgcgtctacttgctcagtcccagctctgcctgttgctctagcccacaggctccctgcggagggtctgggcttggcggaggacccagaatggcactgaggccagcatggctgtgggagttgagcaaaccctgggtgccagtccaggagctgtggctggacatggggtgatggggcgggatgcttgggcctgggtctctccgccagcagtgccaggagcccttgctggggaaatcaagaccagactaggatgcttctgccccaggcctgccttccattctttaacagcccatcttggcttggggttgcaatgatggctgggccagtcacttgtggcagggcatcagggccctggcagggaagaacctaggcacctggggttgtccccagcctgcccgtcagcatgagatacccagtgggaaagtgagaggatgcggagaggttggcagagccaggggtaggttctggaggctcaagcaacaaggaggtgcaggtaaagggtgcagtgcagccactgagggacagctgggaactgggggatgcaagtgagaaagggatgtgggggagagttcaggatcaggctgcttgaggagtaatgggttacctacagcagagacgagatggctgtttgtcaggaggcggtagaaaggtagaaaggattcagattgtggaagggtaaacggatgagatgaccattaaggttttgttttactgagaggctgtaagtctgccagggacagctgtcagtaaggctgcagaagggctggggggctacacaaggaagagcagaactagggttgtaagctcaaatgacgagcaaacggtgagaaggaggcctgaagggctgtgtggggactatggctacctgaagagggcacaccctgttaaaggggccacagctgctcagctgttcttacagtgccggccctgtgttgccagattgtctgctttgtcagaggccagaattctgactttttatgtgaaataggatttttaaatgctggtgatcacttaaaaaaaacttaaaaacccaatactggcaaaagaggatgccagtttgcaatccctgaagtagagagagctcgtgctggggagaagtccaccaagatgctttgaggcgggctgtagcaaagtcctgtttctcagagctgggctgggctggggtaggatccttgcagctgaggaggaggaaaagccactgagtctcctcccagagcgggacaaaccagaggccctttgcagttgctgggtcaccagcgggggtggcgcatggagtacagacagtgtagctcttggcctgccagggagacggatggtgcctttgaagcaaagaggaagggaaggcagaaccagggatgcctttgctgatgagagtgcctgtcagggaaggcgccacaggctggcagctcttcaaaccagcagcgcttgacccagagccagatccagcatggcctggccagaggcaccctgggaggccagctggtcagtcccttgcctccccaagttccccctggggtcaatgagccctgggaggatgcctaacctaactccagccagactaataggggcatggtgacccttgactcaccatcccatcccagctttcagggagtgggggtagtgtggtctccatgttcctactatgcctaagaagagatggctcaccttgggaggtgccaggctgaaactaggtcctttccgggtctggatgctgccgctcaggagcaggggcgtggcctcagctgcctctgggagcttcccgggaatgacagggtttgaggggagtagatatgagagggagccgctcctggttctggagtcttaggaggttccaacttgcaggatcctttcccagagccctccatggagaaaacagcaaaatgaagcccttacctgcttgctgtctgcaagggagggagccgagccccagctgataatcccccagcactcacccttcctgagctgagacttcggggctgtggagaccagcacaggacatagtggtgctttttaaatttatttttaactgtttctcatatgtagcaacccctcctcccctcctgggcatgtttacacaggctctgctctgggggctggcctggctgtgaggtttctggggaggcagagaggcagggactttggggccttagtcaccatccatggtatcacctcatctcacttcctgtgagggacagggcctggctgatgtgatcccagctccccccagttcaggactgtctttcagctcctttgcccctggaggtgggggctgctggctgaggaggggtcaaggtgagttcaagaaagctacctgtggaaaatggaccaggttgggggggtgattgcaaagtctccccaaagcctggctcctcatgctcagtgccaggggcagaacactggggagccaggtatagagagccttcctgtcataactgccagtcctcttcctccaaggcctctgcatattctcatgttcccctcacccatcatgccagccacccctatccctcttctagcagggccaagatggggacagcagcagccctctggccttgggatgtgatgataaagcaagcctagggccagggtttggggagcagagagagccaagaagttgaccacgtgtgatttccagcccttcccactgggacttgacttcccaggtcaaggagtccgtctcattctggctggtcgagtgaccagaggcctgtgtgaatgtgtgcacctgcttttcctgcctggaatgttttctggctcagctgcagcaacatctgtgagcccagtgtctgccctgtgtccctgggctcgctccaagtgcaggaacatacatgcagggcccaacatgatgatggtgtgaagggcaggaaacagtcctctgaaggagtggggaggtgggcagtctgcccccgccaggtaccatcgcctcctgccagcttccttagaccaggcagggctgccatggtgctagctgcaagtccatcagtattgaccgtctcgctccatcttggtcctccggagtcccaagtttccttttcatcaaatctgacaagagagaagaaacatgggtgtgcttggcccacagggcctggtggtgatggacctccccgctccctcaagctctggatggctgcagtgttgtactagactttgttcaggctgttctcatctcagtattgccccttcctttcactttcacacttcatctcattcctgttgtcactttccccgaaacgaataaagtctccccagctcctgctgtgtaggctgggcagaaaccacaa

SEQ ID NO: 57 - protein, 365 aa, Homo sapiens
MPGGKKVAGGGSSGATPTSAAATAPSGVRRLETSEGTSAQRDEEPEEEGEEDLRDGGVPFFVNRGGLPVDEATWERMWKHVAKIHPDGEKVAQRIRGATDLPKIPIPSVPTFQPSTPVPERLEAVQRYIRELQYNHTGTQFFEIKKSRPLTGLMDLAKEMTKEALPIKCLEAVILGIYLTNSMPTLERFPISFKTYFSGNYFRHIVLGVNFAGRYGALGMSRREDLMYKPPAFRTLSELVLDFEAAYGRCWHVLKKVKLGQSVSHDPHSVEQIEWKHSVLDVERLGRDDFRKELERHARDMRLKIGKGTGPPSPTKDRKKDVSSPQRAQSSPHRRNSRSERRPSGDKKTSEPKAMPDLNGYQIRV

SEQ ID NO: 58 - DNA, 4341 bp, Homo sapiens
  CDS (405)..(1340): VASH2 CDS
  misc_RNA (999)..(1019): VASH2 sh1 target
  misc_RNA (1007)..(1027): VASH2 sh2 target
  misc_RNA (1008)..(1028): VASH2 sh3 target
attcggaacatcggcggctgcccgggtgacttggttatatgtcacagaataacattcgagaatgggacatggaatgaggcgacggccccagcaagccccagcagccccagccccagccccaggcccagcagcagcagcagcagcccctgctctcccgccgcccggagaggctctgcagccatgaagccaaccgtcccgaggcaccgccggtaagcagggagcctccgcggagctcccgccgccgctcccccttggtgagtcctgccgcagcgagaggcatggagaaggccgcccccgcggggcgctgatcccctcgccgcgcccgcgcgcacacgccccccgccgccgccgccgctgccgccgccgcgcgcccccagtacctcgctccccgcccaggccccaccatgaccggctccgcggccgacactcaccgctgcccccaccccaaaggcgccaaaggcacccggtcccggagcagccacgcgcggcccgtgagcctcgccaccagcgggggctcagaggaggaggacaaagacggcggggtgctgttccacgtcaacaagagcggcttccccatcgacagccacacctgggagcgcatgtggatgcacgtggccaaggtgcaccctaaggggggagaaatggtgggcgccatcaggaacgccgccttcttggcaaagccttcaataccccaggtcccaaactacaggctgtcgatgacgatcccagactggctccaggcgatccagaattacatgaagaccctacactacttaaccaatgggcagccttccattgagcggttccccatcagctttaaaacctacttctcaggaaactactttcaccacgttgtgctggggatttactgcaatggccgctatggctcattgggcatgagccgcagggctgagctgatggacaagccattgacttttcggactctgagtgacctcatctttgactttgaggactcttacaagaaatacctgcacacagtcaagaaggtcaagattgggctgtacgtcccccatgagcctcatagcttccagcccattgagtggaagcagctggtcctcaacgtctcaaagatgctgagggctgacataaggaaggagctggagaaatatgccagggacatgagaatgaagatcctgaaacctgcaagtgcccactctccgacccaagtgagaagccggggaaaatccctgtcccccagaaggagacaggcaagccccccgaggaggctcggccggcgagagaagtcgcctgcactgcctgaaaagaaggtggctgatctgagcactctgaatgaagtgggctatcaaatccgaatttagccaagccataccggccagcaagagggtttctgtggtgcttctctctgcactttacccagcatcttcaggaggaactgcaactatttattaagaacttgtgaattttatttttaaggattcacctggaaatagaatctgagtgggtggtaaccattagctttaaaaaattcactaaaaggcaccatgaaaaggctgaagtaataagccccactggggttggactatgtccctcactcaagatcttaaggataaccgtaactgaagttttatatttttccatttacctactttcttttacttgctttgaacattatgcctcaccaatagtaaatgttcatgaaataatctcttgaacttttggtatagtaaggtaactctaacagtattactgtctttttcagcaataacagaaagcaaaaatgggtgggttttttttaagcagttattacctcagcattttgacatcagatatgcaaacttaatggcgttttgtttttttatattctatttgtattctttccccagtatttcccatggggatctccacaagtttggagttttttcctggtgcacacacgtgaggagatttaaggtactatatgcaagtgttttactaaaaagcactgaaattcttctggcaatacaagaaccattttcaggatcttggagttacttccttcttaatctttcttaaagcattcactgatgtttttgttttttcaaaatgaaacaaaaatatcacattgagaagctagtctatgttctgtcactaacatttaaactttgcagactctaacaaaaagcacaagaggtcacgtactattatacaaatttagcggtactggatttacctctgacattaacacactcaggcagagaccaggagtgatcagcaggtcttcagaaccaaaaaacctttctgttcacatttcatctgatttttaaactgaggcaggctttgattcttctgaaggatgccaagaatcaaactaagggaggactcactgttaaagatgtgttctgatgtcttatattaagaccaaatgtgacatgatgtgattatcttccagtactttgcttttaggtaccatttcatgacattttaggaatgagtattggaaaatataaagaattagaaaagcagcactttttttttaatggaaaagtcttcggtccagtgttacaccttatagtgtaattcagtccctaagcacagaatgaatgtctggcctgcatatggtagttacagtgtaacctctggctgcagaccacacaggacaaccctaacagcctagtcttgtatggtgtaaatatcaagagtacagcttcaatttcatttgctttatcttagcaacaatgccaactcaggagagcagacggccgatttcagtgaagtctggtagtcaacagatgttatttcagtctcagtgcatctcctctggctttctttgactgaaggtgtttataggaaggaagttaaaaaaaaaaaaagctcattgagattctttaccaattctttacaagatttctgggggtgacagggaaagcaaaaggacttcaaagcaaaagggtgcacaagagcttacttcaccctgaaaatcagtattattaatgaaaagtactgttttcttaaagaagtcgaatgtcctttagatgaacaagaccaagtatacatctccattagattaaaatgtagcacagggttaaaaattatcagtttaatctctttaagaacagtattatcagagtttaaaatgagttagctctgtttatctacacacagcaaacccattcgcagcctcttggccacatgtattcagatgtttgaaatagtgaatcatttcattttcattctaaaacaatactgacttagccatataccttctgtttgtcaatctgaaacttgcttacatctaataagtagacctcttataacactgcaaccattctaagagttggaatttatttttgccaagtattaagtactgttacatctaaaatacagaatgtcaaatggttgcatagcttgtttcccacaacaaggaggaaaagaacaggaatacaaactctgtaatatgctgataaagaagccttagaactgccaactggcttgatggttcaattagtaagctaatttctcccacacccgctccttgatttttagactaattttcccaaagcaaggtttagtcacacaaacttgaaagtacatttaaaatgttcttagttgtcatcctacttttattgcctatggaatatgctaatttctaaaaaaaatacgggagaggcaccaaacccctaaattctagtgcaataatttaaaaaacaaaaaacaaaacaaaacaaaaaacatgttatatgcacccccctcccaaagctgattttaagactggctagaattcaaaatataaaaaataaaactatgaagtgatttgaaatcggttttaaaaagttgttagtgcaagagaaattttatgtttaagccatcatggcaatatatgcaaagtttaaatgaatgacagaaatcttgattttagactgtatgttgtgctgttttgagtacactttatttcggagatatttagtattttctatacactctgaaatcatgagcatttcactttttctaagtcaattatttcataaggattttattaatagatattggtaaatagaactttggaaatcttaattcagaattcttactatacctgagtcttttgtaagctatagttttatgtacaaccttgtacagtttttttgacatacaattcagaactttgtttattctcttggactttgttctggccaatacattttttttaaatctttaagaagaactgtgattgttttagtgggtatttttctaattaaatgaaaacttgtataatggtattttagagaatgccagtaagtgcatgtttcaagttaatgtttttctcatcacttgtatgtttctaacacagcatgggactttaataaaaccatcctggaaacttaa

SEQ ID NO: 59 - protein, 311 aa, Homo sapiens
MTGSAADTHRCPHPKGAKGTRSRSSHARPVSLATSGGSEEEDKDGGVLFHVNKSGFPIDSHTWERMWMHVAKVHPKGGEMVGAIRNAAFLAKPSIPQVPNYRLSMTIPDWLQAIQNYMKTLHYLTNGQPSIERFPISFKTYFSGNYFHHVVLGIYCNGRYGSLGMSRRAELMDKPLTFRTLSDLIFDFEDSYKKYLHTVKKVKIGLYVPHEPHSFQPIEWKQLVLNVSKMLRADIRKELEKYARDMRMKILKPASAHSPTQVRSRGKSLSPRRRQASPPRRLGRREKSPALPEKKVADLSTLNEVGYQIRI

SEQ ID NO: 60 - DNA, 807 bp, Homo sapiens
  CDS (245)..(445): SVBP CDS
  misc_RNA (345)..(365): SVBP sh1 target
  misc_RNA (394)..(414): SVBP sh2 target
  misc_RNA (397)..(417): SVBP sh3 target
ctgcgcacgcgcggccgctcctgcgggcggaagcctcctggcgctcggccctgcgcccctccccccggcgcgcggccccgcccaggacgaggggcggtgggcaggcagcgctgggtccagtggtcgggggaaggcgaggattaccctcccggaggcgttgtctgcgactcggcggaggctccaacttccagtggcccggtcgggaaagatcagagcctcctaagaaatatccagaagtcaagccatggatccacctgcacgtaaagaaaaaaccaaagttaaagaatctgtcagcagagttgagaaggccaaacagaaatcagcccagcaggagctgaagcagagacaaagagcagagatctatgccctcaacagagtcatgacagaactggagcagcagcagtttgatgagttctgtaaacagatgcagcctcctggagaatgaggccccaggttcaaaccagatgggatcctgagagctttgagagggtgttgccagttttaagctgagatggcccatttggagccttacagaactagaccagaagacatttgtggataaatctgaactcacttcctgtgtgtaacttctaacttcactggttcctcccatctgaattggccacatgcttgctgaagagacttgttttcttcttctaagacagtggttttgttttttgggtttttttaaaaaaaaaaaaaaaacacacacaaaaaacactttttctattgtttgagaaaaatcagtgtttctcatgaaactctatatcttctccaaaaatacaaataaaatactaataaaatgtt

SEQ ID NO: 61 - protein, 66 aa, Homo sapiens
MDPPARKEKTKVKESVSRVEKAKQKSAQQELKQRQRAEIYALNRVMTELEQQQFDEFCKQMQPPGE

SEQ ID NO: 62 - DNA, 4476 bp, Artificial Sequence
  vector genome of AAV.cTNT.TTL-IRES-dsRED
  repeat_region (1)..(130): 5′ ITR
  promoter (214)..(655): cTNT promoter
  Intron (772)..(904): chimeric intron
  misc_feature (997)..(2160): TTL
  RBS (2194)..(2778): IRES
  misc_feature (2782)..(3459): DsRED
  misc_feature (3523)..(4110): WRPE
  polyA_signal (4132)..(4258): Rabbit globin polyA
  repeat_region (4347)..(4476): 3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttacgggccccccctcgaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgctaccggactcagatctggtaccatgtacccatacgacgtcccagactacgctatgtacaccttcgtggtacgcgatgagaacagcagcgtctacgccgaggtctcccggctgctcctcgccaccggccactggaagaggctgcggcgagacaaccccagattcaacctgatgctgggagagaggaatcggctgcccttcgggagactgggtcacgagcccgggctggtacagttggtgaattactacaggggtgctgacaaactgtgtcgcaaagcttctttagtgaagctaatcaagacaagccctgaactggctgagtcctgcacatggttccctgaatcttatgtgatttatccaaccaatctcaagactccagttgctccagcacagaatggaattcagccaccaatcagtaactcaaggacagatgaaagagaattctttctcgcctcttataacagaaagaaagaggatggagagggcaacgtttggattgcaaagtcatcagccggtgccaaaggtgaaggcattctcatctcctcagaggcttcagagcttctcgatttcatagacaaccagggccaagtgcacgtgatccagaaatatcttgagcaccctctgctgcttgagccaggtcatcgcaagtttgacatccgaagctgggtcttggtggatcatcagtataatatctacctctatagagagggtgtgcttcggactgcttcagaaccatatcatgttgataatttccaagacaaaacctgccatttgaccaatcactgcattcaaaaagagtattcaaagaactacgggaagtatgaagaaggaaatgaaatgttcttcaaggagttcaatcagtacctaacaagtgctttgaacattaccctagaaagtagtatcttactacaaatcaaacatataataaggaactgcctcctgagcgtggagcctgccattagcaccaagcacctcccttaccagagcttccagctcttcggctttgacttcatggtcgatgaggagctgaaggtgtggctcattgaggtcaacggtgcccctgcatgtgctcagaagctctatgcagaactgtgccaaggcatcgtggacatagccatttccagtgtcttcccacccccagatgtggagcaacctcagacccagccagctgccttcatcaagctgtgactcgagcctaagcttctagataagatgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtctatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcctcgacccgggcggcctcgaggacggggtgaactacgcctgaatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgacgtgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 63 - DNA, 4045 bp, Artificial Sequence
  expression cassette AAV.cTNT.TTL-IRES-dsRED
  promoter (1)..(442): cTNT promoter
  Intron (559)..(691): chimeric intron
  misc_feature (784)..(1947): TTL
  RBS (1981)..(2565): IRES
  misc_feature (2569)..(3246): DsRed
  misc_feature (3310)..(3897): WRPE
  polyA_signal (3919)..(4045): Rabbit globin polyA
cgggccccccctcgaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgctaccggactcagatctggtaccatgtacccatacgacgtcccagactacgctatgtacaccttcgtggtacgcgatgagaacagcagcgtctacgccgaggtctcccggctgctcctcgccaccggccactggaagaggctgcggcgagacaaccccagattcaacctgatgctgggagagaggaatcggctgcccttcgggagactgggtcacgagcccgggctggtacagttggtgaattactacaggggtgctgacaaactgtgtcgcaaagcttctttagtgaagctaatcaagacaagccctgaactggctgagtcctgcacatggttccctgaatcttatgtgatttatccaaccaatctcaagactccagttgctccagcacagaatggaattcagccaccaatcagtaactcaaggacagatgaaagagaattctttctcgcctcttataacagaaagaaagaggatggagagggcaacgtttggattgcaaagtcatcagccggtgccaaaggtgaaggcattctcatctcctcagaggcttcagagcttctcgatttcatagacaaccagggccaagtgcacgtgatccagaaatatcttgagcaccctctgctgcttgagccaggtcatcgcaagtttgacatccgaagctgggtcttggtggatcatcagtataatatctacctctatagagagggtgtgcttcggactgcttcagaaccatatcatgttgataatttccaagacaaaacctgccatttgaccaatcactgcattcaaaaagagtattcaaagaactacgggaagtatgaagaaggaaatgaaatgttcttcaaggagttcaatcagtacctaacaagtgctttgaacattaccctagaaagtagtatcttactacaaatcaaacatataataaggaactgcctcctgagcgtggagcctgccattagcaccaagcacctcccttaccagagcttccagctcttcggctttgacttcatggtcgatgaggagctgaaggtgtggctcattgaggtcaacggtgcccctgcatgtgctcagaagctctatgcagaactgtgccaaggcatcgtggacatagccatttccagtgtcttcccacccccagatgtggagcaacctcagacccagccagctgccttcatcaagctgtgactcgagcctaagcttctagataagatgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtctatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcctcgacccgggcggcctcgaggacggggtgaactacgcctgaatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgacgtgaggatccgatctttttccctctgccaaaaattatggggacatcatgaagccccttgagcatctgacttctggctaataaaggaaatttattttcattgcaatagtgtgttggaattttttgtgtctctcactcg